(19) United States
(12) Patent Application Publication
(10) Pub. No.: US 2023/0263515 A1
(43) Pub. Date: Aug. 24, 2023

(54) IMPLANT ASSEMBLY AND METHOD FOR ACCESSING DURAL VENOUS SINUS OR OTHER FLUIDS INSIDE THE CRANIUM

(71) Applicant: Barry M. FELL, Hummelstown, PA (US)

(72) Inventor: Barry M. FELL, Hummelstown, PA (US)

(21) Appl. No.: 18/112,467

(22) Filed: Feb. 21, 2023

Related U.S. Application Data
(60) Provisional application No. 63/312,164, filed on Feb. 21, 2022.
(60) Provisional application No. 63/404,587, filed on Sep. 8, 2022.
(60) Provisional application No. 63/434,537, filed on Dec. 22, 2022.

(51) Int. Cl. A61B 17/04

(52) U.S. Cl. CPC A61B 17/0401; A61B 2017/0427

(57) ABSTRACT

An implant assembly for accessing a dural venous sinus (DVS) in a cranial bone is configured to be received in a macroaperture created in the cranial bone adjacent an outer table thereof and in a connected microaperture of smaller diameter than the macroaperture created in the cranial bone and extending toward an inner table thereof. The implant assembly includes a bone anchor configured to be received in at least the macroaperture and having a lumen therethrough, and an implant body configured to be received in the lumen of the bone anchor and extend through the bone anchor into the microaperture, the implant body having a lumen for providing access therethrough to the DVS.

### CROSS-REFERENCE TO RELATED APPLICATIONS

This application claims the benefit of U.S. provisional application Ser. No. 63/312,164 filed Feb. 21, 2022; U.S. provisional application Ser. No. 63/404,587 filed Sep. 8, 2022; and U.S. provisional application Ser. No. 63/434,537 filed Dec. 22, 2022, the disclosures of which are hereby incorporated in their entirety by reference herein.

### TECHNICAL FIELD

Embodiments relate to implant assemblies and methods for accessing fluids inside the cranium, such as the dural venous sinus.

### BACKGROUND

Safe and straightforward access to fluid in a dural venous sinus (DVS) and other fluid-containing spaces, such as cerebrospinal fluid (CSF) found in the subarachnoid space, is important in humans and animals for a plurality of therapeutic, diagnostic and monitoring purposes. Exemplary purposes may include introduction of media, electromagnetism, medicine, and other materials to augment or improve function, or to attempt to address or prevent various pathologies. Such pathologies may include, for example, amyotrophic lateral sclerosis (ALS), cancer, various types of damage or degeneration, depression, dystonia and other movement disorders, epilepsy, headache, memory loss, migraine, multiple sclerosis (MS), obesity, pain, Parkinson's Disease, psychiatric disorders, seizure, stroke, tumors, tremor, and/or minimally conscious states, and others. Additional purposes may include removal of media for a variety of purposes, such as to address hydrocephalic conditions, relieve pressure, reduce pain, or to test the fluid. Still further, other purposes may include diagnosis by various methods (e.g., electrical, chemical, or biological), or monitoring one or more conditions of a patient or aspects of their pathologies.

U.S. Pat. No. 9,402,982 to Baert et al. relates to an implantable catheter for insertion through a cranial bone into a venous sinus to drain excess cerebral spinal fluid. Baert et al. utilize an internal-to-the-cranium fixation element or internal stop configured for epidural placement and attachment directly to the soft tissue or bone in the immediate vicinity of the venous sinus. As opposed to a minimally invasive approach, Baert et al. disclose making a burr hole and then finding the target location by direct visualization of the venous sinus. U.S. Pat. No. 9,179,875 to Hua is drawn to insertion of medical devices through non-orthogonal and orthogonal trajectories within a cranium. However, Hua is focused on electrodes or other devices, and maximizing the length of such devices that can be implanted within or through the cranium and their ability to hold charge.

### SUMMARY

In one or more embodiments, an implant assembly for accessing a dural venous sinus (DVS) in a cranial bone is configured to be received in a macroaperture created in the cranial bone adjacent an outer table thereof and in a connected microaperture of smaller diameter than the macroaperture created in the cranial bone and extending toward an inner table thereof. The implant assembly includes a bone anchor configured to be received in at least the macroaperture, the bone anchor having an upper portion with a larger diameter than a lower portion thereof, the bone anchor having a lumen therethrough. An implant body is configured to be received in the lumen of the bone anchor and extend through the bone anchor into the microaperture, the implant body having an upper portion with a larger diameter than a lower portion thereof, the implant body having a lumen for providing access therethrough to the DVS. An inner surface of the bone anchor and an outer surface of the implant body have complementary shapes to secure the implant body to the bone anchor via an interference fit.

In one or more embodiments, the bone anchor may have a substantially constant wall thickness along a length thereof.

In one or more embodiments, an inner surface of the bone anchor may include a shelf configured to receive the upper portion of the implant body thereon.

In one or more embodiments, a distal tip of the implant body may extend beyond a distal end of the bone anchor when the implant body is received in the bone anchor.

In one or more embodiments, a lower portion of the implant body may include at least one port adjacent the distal tip.

In one or more embodiments, the distal tip of the implant body may include a sensor.

In one or more embodiments, the bone anchor may include a top portion configured to receive a sensor.

In one or more embodiments, the implant body may have a top portion with slots for facilitating insertion, positioning, and removal of the implant body in the bone anchor.

In one or more embodiments, an external shape of the implant assembly may be complementary to a shape of the macroaperture and microaperture.

In one or more embodiments, the bone anchor may have an outer surface which includes at least one engaging member configured to engage with the macroaperture.

In one or more embodiments, the implant assembly may include a resealable membrane configured to be received on the implant assembly, wherein the lumen of the implant body is accessible therethrough.

In one or more embodiments, the implant assembly may include a fluid coupler configured to be received on a proximal end of the implant assembly and operably connected to a catheter for fluid flow between a fluid source and the implant assembly.

In one or more embodiments, the bone anchor may include a conduit integrally formed therewith for fluid flow between a fluid source and the implant assembly.

In one or more embodiments, the implant assembly may include a cap configured to be received in a proximal opening of the bone anchor, the cap including a lumen and a plurality of orifices of different dimensions in fluid communication with the lumen, wherein the lumen of the bone anchor is accessible via the cap and the cap is configured to be rotatable so that one of the plurality of orifices aligns with the conduit to select a desired fluid flow rate through the implant assembly.

In one or more embodiments, the implant assembly may be constructed from at least one of a metal, plastic, ceramic, resorbable compound, biologic tissue, bone substitute, or material derived from a cadaver.

In one or more embodiments, the implant assembly may be coated with a low friction antithrombogenic material or a material which limits bony or other tissue ingrowth.

In one or more embodiments, an implant assembly for accessing a dural venous sinus (DVS) in a cranial bone is configured to be received in a macroaperture created in the cranial bone adjacent an outer table thereof and a connected microaperture of smaller diameter than the macroaperture created in the cranial bone and extending toward an inner table thereof. The implant assembly includes a generally cylindrical bone anchor configured to be received in the macroaperture, the bone anchor having external threads configured to engage the macroaperture, the bone anchor having a lumen with internal threads. An implant body is configured to be received in the lumen of the bone anchor and extend through the bone anchor into the microaperture, the implant body having external threads configured to be received by the internal threads to locate and secure the implant body in the bone anchor, the implant body having a lumen for providing access therethrough to the DVS.

In one or more embodiments, the bone anchor may have an internal stop surface configured to engage the implant body when received in the bone anchor, the internal stop surface having an opening therein for receiving the implant body therethrough.

In one or more embodiments, the implant body may have a proximal portion and an elongated distal portion, wherein the distal portion has a diameter smaller than a diameter of the proximal portion, wherein when the implant body is received in the bone anchor, the proximal portion is configured to engage the internal stop surface and the distal portion is configured to be received through the opening.

In one or more embodiments, the implant body may be tubular with a constant cross-sectional area, the implant body having a stop plate configured to engage the internal stop surface of the bone anchor when the implant body is received in the bone anchor.

In one or more embodiments, the implant assembly may include a hollow needle arranged to be received through the implant assembly, the needle including a sharp distal tip, markings to indicate depth of insertion, and a stop member configured to engage the implant assembly to limit depth of insertion of the needle through the implant assembly.

In one or more embodiments, a proximal end of the implant body may include a connector configured to be coupled to a catheter or a fluid coupler.

In one or more embodiments, an implant assembly for accessing a dural venous sinus (DVS) in a cranial bone is configured to be received in a macroaperture created in the cranial bone adjacent an outer table thereof and a connected microaperture of smaller diameter than the macroaperture created in the cranial bone and extending toward an inner table thereof. The implant assembly includes a generally cylindrical bone anchor configured to be received in the macroaperture, the bone anchor having external threads configured to engage the macroaperture, the bone anchor having a lumen with internal facets. A generally cylindrical implant body is configured to be received in the lumen of the bone anchor and extend through the bone anchor into the microaperture, the implant body having external facets configured to be received by the internal facets of the bone anchor such that the implant body is movable with respect to the bone anchor in fixed angular steps, the implant body having a lumen for providing access therethrough to the DVS.

In one or more embodiments, the bone anchor may have an upper flange configured to limit a depth of insertion of the bone anchor in the cranial bone.

In one or more embodiments, the bone anchor may include index marks thereon.

In one or more embodiments, the lumen of the implant body may have a longitudinal axis that is offset from a longitudinal axis of the implant body, the implant body having index marks thereon for aligning the implant body with the bone anchor.

In one or more embodiments, the implant assembly may include an indexing tool having external facets configured to be received by the internal facets of the bone anchor, a lumen extending therethrough with a longitudinal axis that is offset from a longitudinal axis of the indexing tool, and index marks thereon for aligning the indexing tool with the bone anchor, the indexing tool movable with respect to the bone anchor in fixed angular steps.

In one or more embodiments, the implant assembly may include a locking ring for securing the implant body to the bone anchor.

In one or more embodiments, an implant assembly for accessing a dural venous sinus (DVS) in a cranial bone is configured to be received in a macroaperture created in the cranial bone adjacent an outer table thereof and a connected microaperture of smaller diameter than the macroaperture created in the cranial bone and extending toward an inner table thereof. The implant assembly includes a generally cylindrical bone anchor configured to be received in the macroaperture, the bone anchor having external threads configured to engage the macroaperture, the bone anchor having a lumen with a proximal internal portion and a distal internal portion, the proximal internal portion having a diameter larger than a diameter of the distal internal portion to define a shoulder therebetween. An implant holder is configured to be received in the lumen of the bone anchor, the implant holder including an upper flange configured to engage and be movable along the shoulder such that the implant holder is infinitely rotatable with respect to the bone anchor, the implant holder having a lumen extending therethrough. An implant body is configured to be received in the lumen of the implant holder and extend through the implant holder into the microaperture, the implant body having a lumen for providing access therethrough to the DVS.

In one or more embodiments, the lumen of the implant holder may have a longitudinal axis that is offset from a longitudinal axis of the bone anchor.

In one or more embodiments, the distal internal portion of the bone anchor may have internal facets and the proximal internal portion of the bone anchor has a smooth surface.

In one or more embodiments, the implant holder may include an internal locking cam and associated compression pin, wherein engagement of the locking cam displaces the pin outwardly to engage the proximal internal portion of the bone anchor and fix a position of the implant holder with respect to the bone anchor.

In one or more embodiments, the lumen of the implant holder may be threaded and the implant body may be threaded.

In one or more embodiment, the implant assembly may include a threaded cutting guide configured to be received in the threaded lumen of the implant holder.

### BRIEF DESCRIPTION OF THE DRAWINGS

FIG. 1A is a side view illustrating an embodiment of an implant assembly and method for accessing fluids inside the cranium wherein a combined macroaperture and connected microaperture are created in the cranial bone, a bone anchor is inserted into the macroaperture, an implant body is inserted into the bone anchor and through the microaperture, and then the implant body is further moved into a fluid-containing space found under that location of the cranial bone;

FIGS. 1B and 1C are cross-sectional views illustrating an implant assembly comprising an assembled bone anchor and implant body disposed within the combined macroaperture and microaperture created in the cranial bone with a fluid coupler attached thereto;

FIGS. 1D and 1E are side views illustrating an embodiment in which a combined macroaperture and microaperture are created in the cranial bone for precision placement and holding of the implant assembly;

FIG. 2 is a cross-sectional view illustrating an embodiment in which CSF sourced via a first implant assembly is drained into a DVS via a second implant assembly;

FIGS. 3A-3F are cross-sectional views of a portion of a cranial bone illustrating variations in shape of a combined macroaperture and microaperture according to one or more embodiments;

FIG. 4A is a side view of a portion of cranial bone illustrating a combined macroaperture and microaperture according to one or more embodiments, an outer portion of which has a hexagonal cross-section;

FIG. 4B is a top view of the portion of the cranial bone of FIG. 4A;

FIG. 5A is a side view of a portion of cranial bone illustrating a combined macroaperture and microaperture according to one or more embodiments, an outer portion of which is star-shaped;

FIG. 5B is a top view of the portion of the cranial bone of FIG. 5A;

FIG. 6A is a side view of a portion of cranial bone illustrating a combined macroaperture and microaperture according to one or more embodiments, an outer portion of which has an oval cross-section;

FIG. 6B is a top view of the portion of the cranial bone of FIG. 6A;

FIG. 7A is a cut-away, perspective view of a long offset bone anchor according to one or more embodiments;

FIG. 7B is a perspective view of an offset implant body configured to be received inside the bone anchor of FIG. 7A according to one or more embodiments;

FIG. 7C is a cut-away, perspective view of the offset implant body of FIG. 7B;

FIG. 7D is a cut-away, perspective view of an implant assembly comprising the offset implant body of FIGS. 7B and 7C positioned within the long offset bone anchor of FIG. 7A;

FIG. 8A is a perspective view of a short non-offset bone anchor according to one or more embodiments;

FIG. 8B is a cut-away, perspective view of the short non-offset bone anchor of FIG. 8A;

FIG. 8C is a cut-away, perspective view of an implant assembly comprising the short non-offset bone anchor of FIGS. 8A and 8B with a long cylinder implant body therein according to one or more embodiments;

FIG. 8D is a cut-away, perspective view of the implant assembly of FIG. 8C with a membrane thereon according to one or more embodiments;

FIG. 9A is a cut-away, perspective view of a combined macroaperture and microaperture and a channel in a section of cranial bone according to one or more embodiments;

FIG. 9B is a cut-away, perspective view of a section of cranial bone illustrating the positioning of a short non-offset bone anchor configured as in FIGS. 8A and 8B;

FIG. 9C is a cut-away, perspective view of a section of cranial bone illustrating the positioning of an implant assembly configured as in FIG. 8D;

FIGS. 10A and 10B are a cut-away, perspective views a combined macroaperture and microaperture and a channel in a section of cranial bone according to one or more embodiments;

FIG. 10C is a cut-away, perspective view of a section of cranial bone illustrating the positioning of a long offset bone anchor configured as in FIG. 7A;

FIGS. 10D and 10E are cut-away, perspective views of a section of cranial bone illustrating the positioning of an implant assembly configured as in FIG. 7D;

FIG. 11A is a cut-away, perspective view of a combined macroaperture and microaperture and a channel in a section of cranial bone according to one or more embodiments;

FIG. 11B is a cut-away, perspective view of a section of cranial bone illustrating the positioning of a short offset bone anchor according to one or more embodiments;

FIGS. 11C and 11E are cut-away, perspective views of a section of cranial bone illustrating the positioning of an implant assembly comprising the bone anchor of FIG. 11B and a long offset implant body according to one or more embodiments;

FIG. 11D is an enlarged view of the implant assembly of FIG. 11C;

FIGS. 12A and 12B are perspective views of a combined macroaperture and microaperture and a channel in a section of cranial bone according to one or more embodiments;

FIG. 12C is a cut-away, perspective view of a section of cranial bone illustrating the positioning of a long non-offset bone anchor according to one or more embodiments;

FIGS. 12D and 12E are cut-away, perspective views of a section of cranial bone illustrating the positioning of an implant assembly comprising the bone anchor of FIG. 12C and a long cylinder implant body according to one or more embodiments;

FIGS. 13A-13F are perspective views of various configurations of implant assemblies according to one or more embodiments;

FIGS. 14A-14M are perspective views of various configurations of bone anchors according to one or more embodiments;

FIGS. 14N and 140 illustrate a bone anchor with a selectable orifice size to control CSF flow and pressure according to one or more embodiments;

FIG. 14P is a cut-away side view of a combined macroaperture and microaperture according to one or more embodiments to be used with the bone anchor of FIG. 14F;

FIGS. 15A-15C are cut-away, perspective views illustrating three variations of ports found at a distal tip of the implant body according to one or more embodiments;

FIG. 16A is a photograph showing the preparation of the cranial bone to accept an implant assembly;

FIG. 16B is a photograph of an implant assembly positioned within the macroaperture and microaperture with a CSF-containing catheter contained within a channel made in the cranial bone;

FIG. 17 depicts a method utilizing a CT, MRI, US or other three-dimensional volume imaging procedure for identifying a desired location for the implant assembly according to one or more embodiments;

FIG. 18 is a schematic representation of one type of kit which may be used with the implant assembly and method according to one or more embodiments;

FIG. 19 is a schematic representation of a second type of kit which may be used with the implant assembly and method according to one or more embodiments;

FIG. 20 is a side view of an implant assembly according to one or more embodiments showing a bone anchor, an implant body, and a fluid coupler for attachment to a catheter;

FIG. 21A is a cross-sectional, exploded view of an implant assembly according to one or more embodiments showing the use of an inline check valve in the fluid coupler and penetration of the distal end of the implant body through the superior dura of the target DVS;

FIG. 21B is a cross-sectional view of the implant assembly of FIG. 21A;

FIG. 22A is a top perspective view of a bone anchor according to one or more embodiments;

FIG. 22B is a top view of the bone anchor of FIG. 22A illustrating interface surfaces and external threads for engaging with the cranial bone according to one or more embodiments;

FIG. 22C is a cross-sectional view of the bone anchor of FIG. 22A;

FIG. 22D is a bottom view of the bone anchor of FIG. 22A;

FIG. 23A is a top perspective view of an implant body prior to insertion into a receiving bone anchor;

FIG. 23B is a bottom side perspective view of an implant assembly with the implant body received in the bone anchor;

FIG. 24 is a side view of an implant body according to one or more embodiments;

FIG. 25A is an exploded view of the bone anchor secured in the cranial bone and the implant body before being received in the bone anchor, with a hollow stop needle configured to be received through a lumen of the positioned implant body to penetrate the dura;

FIG. 25B is a cross-sectional view of the bone anchor in the cranial bone with the implant body partially threaded into the bone anchor but not touching the dura;

FIG. 25C is a cross-sectional view depicting the stop needle received in the lumen of the implant body and just touching the dura;

FIG. 25D is a cross-sectional view of the implant body having been further advanced into the bone anchor and positioned to a positive depth indication as shown on the stop needle;

FIG. 25E is a cross-sectional view of the stop needle advanced through the dura until the stop member engages the proximal surface of the implant body, at which point the downward travel of the stop needle is halted and a positive blood or CSF flash is noted;

FIG. 25F is a cross-sectional view of the implant body having been advanced to its full travel in the bone anchor and having also penetrated the just-opened dura;

FIG. 25G is a cross-sectional view of a final position of the implant body past the dura and with the stop needle being withdrawn;

FIG. 25H is a cross-sectional view of an implant assembly comprising the implanted bone anchor and an implant body and a fluid coupler connected to a catheter carrying CSF or other fluids;

FIG. 25I is a cross-sectional view of an implant assembly having an alternative fluid coupler for engaging with the implant body;

FIG. 26A is a cross-sectional view of an implant assembly with a tubular implant body according to one or more embodiments;

FIG. 26B is a side view of the implant assembly of FIG. 26A;

FIG. 27 shows a side view of the implant body including a stop member of FIGS. 26A-26B;

FIG. 28 is a top perspective view of the implant body of FIGS. 26A-26B and 27 positioned through the bone anchor;

FIG. 29 is a top perspective view of the implant body and bone anchor of FIG. 28 with a locking nut securely tightening the stop member to the bone anchor to lock the implant body in position;

FIG. 30 is a top perspective view of a bone anchor according to one or more embodiments;

FIG. 31 is a cut-away, perspective view of another bone anchor according to one or more embodiments positioned in the cranial bone;

FIG. 32 is a perspective view of the bone anchor of FIGS. 30-31 positioned in the cranial bone and an indexing tool with an external faceted surface for use therewith;

FIG. 33 is a bottom perspective view of the bone anchor of FIGS. 30-31 with the indexing tool therein and a probe received through the indexing tool;

FIG. 34 is a perspective view of the bone anchor positioned in the cranial bone with the indexing tool received therein to be used in locating the longitudinal axis of the DVS in conjunction with the received probe;

FIG. 35 is a top view of the bone anchor within the macroaperture in the cranial bone;

FIG. 36 is a perspective view of an indexable implant body prior to being received by the bone anchor of FIGS. 30-31 positioned within the macroaperture formed in the cranial bone;

FIG. 37 is a perspective view showing the indexable implant body received in the bone anchor, the indexable implant body including a distal tip insertable through the created microaperture and through the dura into the DVS;

FIG. 38 is a side view of the indexable implant body;

FIG. 39 is a cut-away, perspective view of the indexable implant body of FIG. 38;

FIG. 40 is a cut-away, perspective view of the indexable implant body received in the bone anchor;

FIG. 41 is a cut-away, perspective view of the indexable implant body received in the bone anchor, wherein the bone anchor is positioned in the macroaperture in the cranial bone;

FIG. 42 is a perspective view of a locking ring configured to hold the indexable implant body in the bone anchor according to one or more embodiments;

FIGS. 43 and 44 are side and top perspective views, respectively, of the indexing tool within the bone anchor, the indexing tool having a probe received therein;

FIG. 45 is a cross-sectional view of the bone anchor and received indexable implant body penetrating the inner table and dura into the DVS;

FIGS. 46 and 47 show a side view and bottom view, respectively, of the indexable implant body received in the bone anchor;

FIG. 48 is a top view of the bone anchor showing an overlaid schematic map of how the indexing tool and the indexable implant body can be moved within the bone anchor in fixed steps to create a microaperture in a specific location within the area of the macroaperture, wherein the map demonstrates how the origin point of the bone anchor can be offset by a set amount resulting in a curved, laterally displaced microaperture position over the DVS in order to more precisely position the microaperture over the ideal target zone of the DVS;

FIG. 49 is a top view of the bone anchor showing the map overlaid over the location of the DVS with the ideal target zone of the DVS shown along the longitudinal axis of the DVS;

FIG. 50 is a top view of the bone anchor showing the position of the intended microaperture when the indexable implant body is in position “2” of the bone anchor;

FIG. 51 is a top view of the bone anchor showing the position of the ideal target zone for the DVS in reference to the selected position of the microaperture;

FIG. 52 is a top view of the bone anchor showing the position of the indexable tool in position “2” in reference to the intended microaperture location and the ideal target zone of the DVS;

FIG. 53 is a side view showing the indexable implant body with its distal tip passing through the ideal target zone of the DVS;

FIG. 54 is a side view of the bone anchor and received indexable implant body with its distal tip passing into the ideal target zone of the DVS;

FIG. 55 is a top view of the bone anchor showing the position of the intended microaperture when the indexable implant body is in position “6” of the bone anchor, resulting in a poorly placed microaperture that misses the DVS entirely;

FIG. 56 is a top view of the bone anchor and indexable implant body when the indexable implant body is in position “6” of the bone anchor;

FIG. 57 is a side view of the bone anchor and received indexable implant body with its distal tip missing the DVS;

FIG. 58 is a top view of the bone anchor and received indexing tool when the indexing tool is in position “7” of the bone anchor, missing the DVS;

FIGS. 59-61 are top views of the bone anchor and received indexing tool showing the outcome of marginally placed locations of the microaperture (e.g. corresponding to positions “1”, “10”, and “12” of the bone anchor) that miss a portion of the ideal target zone of the DVS;

FIG. 62 is a top perspective view of a bone anchor according to one or more embodiments;

FIG. 63 is a cut-away, perspective view of the bone anchor of FIG. 62;

FIG. 64 is a perspective view of the bone anchor of FIG. 62 with a rotational implant holder received therein;

FIG. 65 is a cross-sectional view of the rotational implant holder depicting its threaded lumen and locking cam;

FIG. 66 is a cross-sectional view of the rotational implant holder of FIG. 65 received in the bone anchor of FIG. 62;

FIGS. 67 and 68 are perspective and side views, respectively, of the probe received within the offset lumen in the rotatable implant holder, rotated in position until the probe is positioned in a desired ideal location over the DVS;

FIG. 69 is a cut-away, perspective view of the probe received within the offset lumen of the rotatable implant holder, rotated in position until the probe is positioned in a desired ideal location over the DVS;

FIG. 70 is a top view showing the rotatable implant holder positioned over the ideal location of the DVS where it may be locked in place with the locking cam;

FIG. 71 is cross-sectional view of a cutting guide received within the offset lumen of the rotatable implant holder;

FIGS. 72 and 73 are perspective and cross-sectional views, respectively, of a cutting guide received within the threaded lumen of the rotatable implant holder, and a cutting device received within the cutting guide to penetrate the remaining inner table and dura of the DVS at the desired microaperture opening;

FIG. 74 is a cross-sectional view of the rotatable implant holder received within the bone anchor;

FIG. 75 shows an implant body according to one or more embodiments configured to be received within the rotatable implant holder;

FIGS. 76 and 77 are perspective and cross-sectional views of the implant body of FIG. 75 in relation to the rotatable implant holder before entry into the ideal location and depth on the DVS;

FIGS. 78 and 79 are cross-sectional and cut-away perspective views, respectively, of the implant body of FIG. 75 in relation to the rotatable implant holder after entry into the ideal location and depth on the DVS;

FIG. 80 is a perspective view of a bone anchor according to one or more embodiments;

FIG. 81 is a perspective view of an implant holder configured to be received in the bone anchor of FIG. 80;

FIG. 82 is a cut-away, perspective view of an implant assembly according to one or more embodiments, wherein the implant holder is received in the bone anchor and an implant body with a connected catheter is arranged for insertion into an opening in the implant holder;

FIG. 83 is a cut-away, perspective view of the implant assembly with the implant body received in the implant holder; and

FIG. 84 is a cut-away, perspective view of the implant assembly of FIG. 83 with a catheter and resealable membrane engaged thereon.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

It should also be understood that while exemplary terminology has been used herein, for the purpose of describing aspects and embodiments of the invention, such terminology is not intended to limit the scope of the invention. It will be apparent to one of ordinary skill in the art that various features, structures, process steps or characteristics described herein may be combined. While some illustrative embodiments are described as having only some features, combinations of features of various embodiments are meant to be within the scope of the present invention, and thereby constitute different combinations as would be understood by those skilled in the art. Accordingly, it is understood that components, dimensions, materials, functionality, and methods described with reference to one implant assembly may be applicable to other implant assemblies disclosed herein. As used herein, the singular forms “a”, “an”, and “the” may encompass both singular and plural, unless the context clearly indicates otherwise.

A portion of a cranial bone has a dural venous sinus (DVS). In humans, the DVS is a venous channel located between the endosteal and meningeal layers of dura mater in a brain. The DVS is also referred to as a dural sinus, cerebral sinus, or cranial sinus. Embodiments disclosed herein provide safe and straightforward access to a fluid-containing space, such as a DVS 2 or subarachnoid space, on and through a cranial bone 1 while minimizing and controlling any unintentional bleeding or cerebrospinal fluid (CSF) loss. Disclosed embodiments may control the direction, pressure and amount of fluid flow for purposes of affecting a disease state, such as hydrocephalus. While accessing a DVS 2 has previously required major surgery, embodiments disclosed herein permit such access to potentially be practiced as an outpatient procedure.

Embodiments disclosed herein utilize a connected and combined macroaperture 3 and microaperture 4 which comprise a stepped channel through a cranial bone 1 to a DVS 2 or another space containing CSF, to guide safe insertion, intimate fitting, and retention of an implant assembly in the cranial bone 1. In one or more embodiments, a diameter of the macroaperture 3 is greater than a diameter of the microaperture 4. When inserted into the combined macroaperture 3 and microaperture 4, the implant assembly provides access from outside the cranial bone 1 to the fluid. The macroaperture 3 and microaperture 4 are dimensioned and configured to guide the implant assembly to a desired location, trajectory, orientation, and starting and stopping points for precise placement and holding of the implant assembly in the cranial bone 1. The combined macroaperture 3 and microaperture 4 are created to have a shape which is complementary to an external shape of the implant assembly, such that the implant assembly is intimately fit within the macroaperture 3 and microaperture 4 to limit depth and rotation, and to establish and maintain the position and fixation of the implant assembly in the cranial bone 1. The guiding combination macroaperture 3 and microaperture 4 provide safe access to the DVS 2 so as to minimize and control bleeding therethrough until the implant assembly is secured in the cranial bone 1.

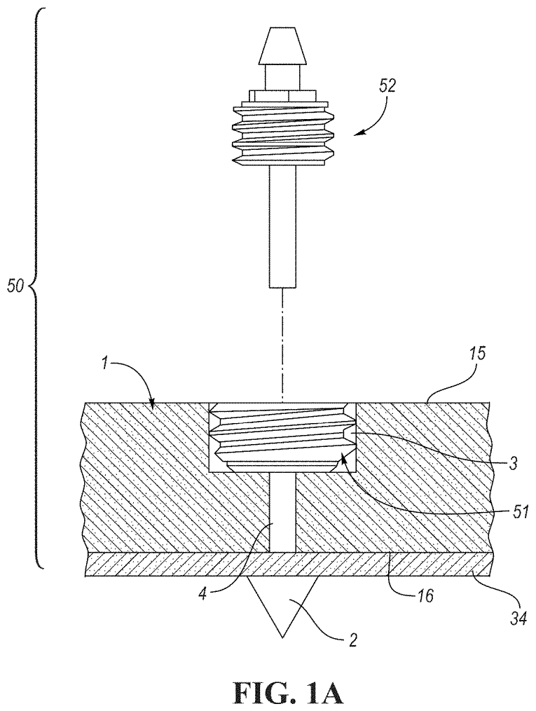

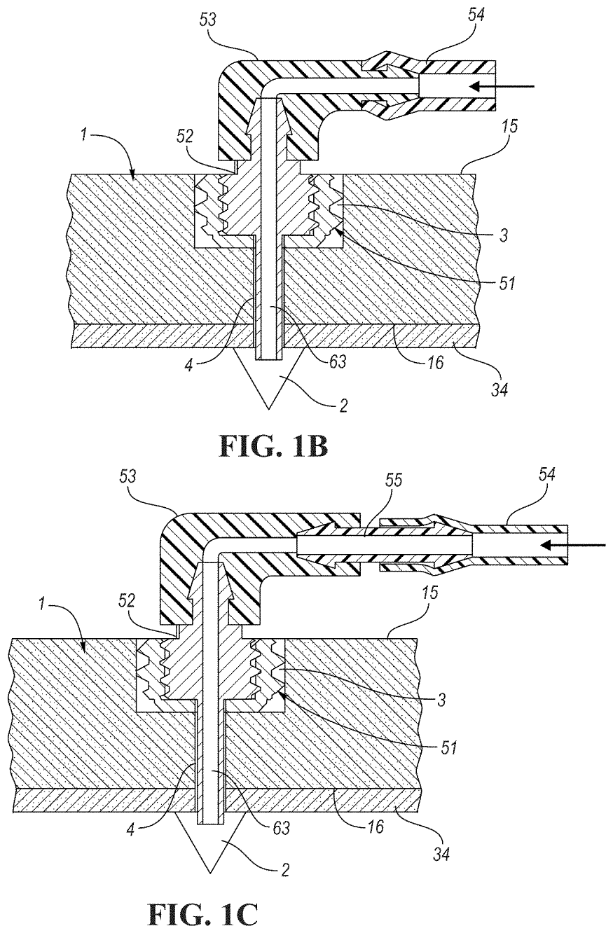

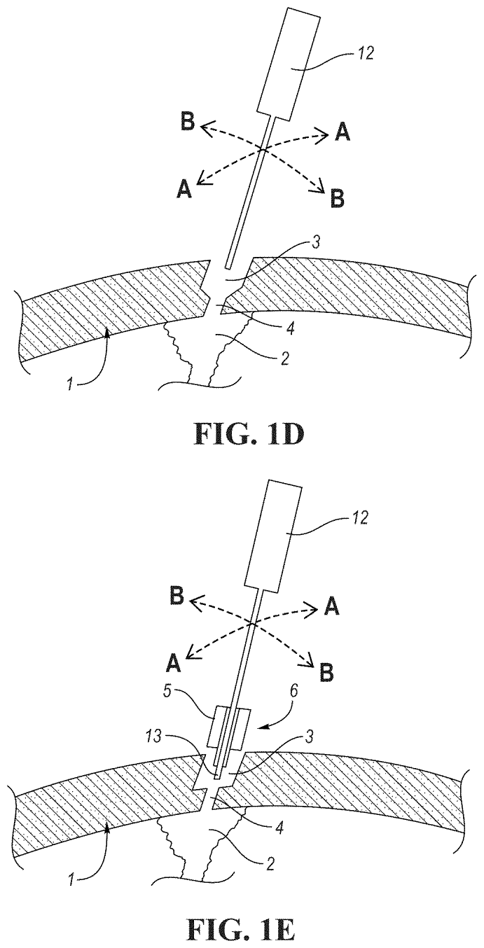

An implant assembly 50 and method for use thereof according to one or more embodiments are illustrated in FIGS. 1A-1C. A combination macroaperture 3 and microaperture 4 in a cranial bone 1 (of a human or non-human animal) are created and used to safely guide insertion and securing of the implant assembly 50. The implant assembly 50 may include a bone anchor 51 configured to be received in a guiding macroaperture 3 created in the cranial bone 1, wherein the bone anchor 51 is configured to receive and internally secure an implant body 52 to be positioned through a corresponding guiding microaperture 4. In one or more embodiments, when inserted into the macroaperture 3, the implant assembly 50 allows a more precise, guiding microaperture 4 to be created within the boundaries of the macroaperture 3 in the cranial bone 1. The implant body 52 is configured to be received within the bone anchor 51 to provide fluid access from outside the cranial bone 1 to the internal fluid-containing space (exemplary fluid flow denoted by arrow).

In FIGS. 1D and 1E, the dashed arcs A-A and B-B illustrate how the trajectory of the macroaperture 3 and microaperture 4 creation may be adjusted with reference to another implant assembly 6 according to one or more embodiments. The combination macroaperture 3 and microaperture 4 may be created first and then an implant assembly 6 inserted therein, or the combination macroaperture 3 and microaperture 4 may be created at least partially simultaneously with the insertion of the implant assembly 6. With reference to FIG. 1E, substantially simultaneous creation of the macroaperture 3 and microaperture 4 and insertion of the implant assembly 6 may be accomplished by advancing a hollow trocar 12 carrying a bone anchor 5 toward and into the cranial bone 1 while a cutting device 13 (e.g. laser, drill and drill bit, grinder, burner, etc.; not shown) passing through the trocar 12 and the bone anchor 5 cuts the cranial bone 1. It is understood that the term “cutting” may be used herein to encompass any method for creating the macroaperture 3 and microaperture 4.

In one or more embodiments, a guiding device such as a robotic arm, stereotactic frame, or navigation-guided CT or MM scan may be used for guiding creation of the combination macroaperture 3 and microaperture 4 at a precise desired location, trajectory, orientation, starting and stopping points, and shape in the cranial bone 1. Furthermore, while embodiments disclosed herein may include manual placement of the implant assembly, this may be difficult for a surgeon due to the small dimensions of the components. Consequently, it is contemplated that such a guiding device may also be used for inserting the implant assembly into the macroaperture 3 and microaperture 4. Prior to creation of the macroaperture 3 and microaperture 4, the cranial bone 1 may be analyzed using an imaging device such as, but not limited to, magnification, X-rays, fluoroscopy, ultrasound (US), computed tomography (CT), magnetic resonance imaging (MM), or nuclear medicine scans, chemical analysis, and assessments of hardness and/or shape. The location of the macroaperture 3 and microaperture 4 may be determined with the help of such imaging devices or with reference to anatomical landmarks (e.g. extending a string line from the nasium to the inion to determine the midline of the sagittal plane and generally a 5-10 cm distance from the nasion). Imaging devices may also be used during insertion of the implant assembly for imaging the components to provide additional guidance and to ensure a proper fit.

It is contemplated that fiducials may be used to assist in identifying the ideal location for and providing guidance in the creation of the macroaperture 3 and microaperture 4. Such fiducials may be used with a computer, software and user interface (e.g. GUI) to control the creation of a macroaperture 3 and microaperture 4 of various shapes, with a dimensional system for properly referencing a patient's anatomy to a surgical navigation system, and for using the surgical navigation system to guide creation of the macroaperture 3 and microaperture 4. In some instances, it may be advantageous to create more than one macroaperture 3 and microaperture 4 in the cranial bone 1 for the purpose of providing the surgeon with real-time feedback (e.g. using a fluoroscope) in order to create a more accurate map of the DVS 2 and identify an ideal location for the guiding macroaperture 3 and microaperture 4.

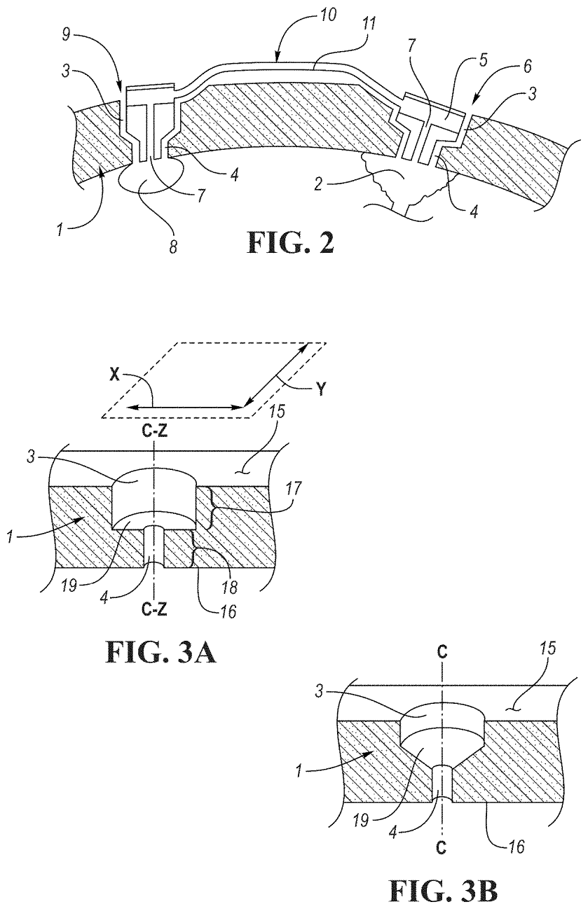

FIG. 2 illustrates an embodiment for draining CSF to a DVS 2, wherein a combination macroaperture 3 and microaperture 4 extend through a cranial bone 1 to a CSF-containing region 8. As shown, an implant assembly 6 and a second implant assembly 9 (e.g., a CSF implant assembly) may be employed. The second implant assembly 9 may be very similar to the implant assembly 6, but could alternatively include a tipped catheter (with side openings) slipped into the CSF-containing region 8 (e.g. the lateral ventricles or cisterna magna or subarachnoid space). The second implant assembly 9 and the implant assembly 6 can function together to provide safe access to the CSF-containing region 8 and minimize and control flow of CSF therethrough.

The second implant assembly 9 and the implant assembly 6 are both configured to be connected to a fluid conductor 10 (or catheter 54) which may take the form, for example, of a tube, duct, pipe, or cannula. Thus, CSF may flow from the CSF-containing region 8, through the second implant assembly 9, through the fluid conductor 10, and through the implant assembly 6 to a DVS 2. This arrangement has many notable advantages, including being safe, straightforward to implement, minimally invasive, and overcoming many of the disadvantages of previous methods of draining CSF. For example, many prior technologies drain CSF to a lower point within the body a substantial distance from the source of the CSF (e.g. to the chest or abdomen), thus making them susceptible to issues such as siphoning and resultant overdrainage of the CSF caused by the substantial hydrostatic pressure created by the height difference between the CSF inlet and outlet. Embodiments disclosed herein avoid such issues caused by hydrostatic pressure. In one or more embodiments, a lumen 7 of the implant assembly 6 may have a smallest cross-sectional area which is smaller than a smallest cross-sectional area of a lumen 11 of the fluid conductor 10, thereby facilitating convenient adjustment of fluid pressure and/or flow rates by changing or switching a cross-sectional diameter of the implant assembly 6.

In one or more embodiments, the macroaperture 3 and the microaperture 4 may be substantially cylindrical with a circular cross-section, with the macroaperture 3 having a diameter of about 3 mm and the microaperture 4 having a diameter of about 1 mm. In one or more embodiments, the smallest diameter of the macroaperture 3 and microaperture 4 may be about 0.25 mm to about 2 mm. In those instances where the macroaperture 3 and microaperture 4 are not circular in cross-section, the smallest cross-sectional area for each may be about 0.05 mm² to about 12.6 mm². In one or more embodiments, the macroaperture 3 may be formed at a preset depth of approximately 2.0 mm to 5.0 mm to accommodate the bone anchor 5, followed by a 1 mm+/−0.5 mm diameter microaperture 4 extending from a bottom of the macroaperture 3 to the interface of the inner table 16 of the cranial bone 1 and the dura 34. Of course, it is understood that these dimensions are merely exemplary, and are not intended to be limiting.

The dimensions of the microaperture 4 may be chosen according to a desired flow rate (e.g. which may be measured in ml of fluid drained per minute, typically in the range from 0.01 to 0.2 ml/min) through the microaperture 4 without building up an excessively high fluid back pressure. High back pressure can be relieved by either creating more than one microaperture 4 through the cranial bone 1 into the DVS 2, or by increasing the internal diameter and/or length of the microaperture 4.

In one or more embodiments, the bone anchor 5 defines an outer surface with a smallest cross-sectional area from about 0.05 mm² to about 12.6 mm². In embodiments where the outer surface of the bone anchor 5 is generally cylindrical, a smallest diameter of the bone anchor 5 may be from about 0.25 mm to about 2 mm. In embodiments where the bone anchor 5 defines a substantially cylindrical lumen 21 therethrough, a diameter of the lumen 21 may be from about 0.1 mm to about 3 mm, or from about 0.75 mm to about 1 mm, depending on circumstances and specific patient needs. Again, these dimensions are merely exemplary, and are not intended to be limiting.

In certain circumstances, it may be sufficient to determine the desired location on the cranial bone 1 that is immediately above the desired DVS 2 or fluid-containing area by image guidance, anatomical landmarks, or both. A tripod guide or similar device may be used to orient a cutting device 13, or a cutting guide attached to a stereotactic frame or robot arm may be arranged to be orthogonal to the surface of the cranial bone 1 before removing bone using a drill or other cutting device, such as a laser or burr. When using a drill and drill bit as the cutting device, to prevent the drill bit from moving too far through the cranial bone 1, the drill bit may have a length which does not extend substantially beyond the cranial bone 1. A depth-controlled drill equipped with a drill stop or cortical bone sensor (e.g. McGinley Orthopedics, Casper, Wyo.) or a pre-programmed drill depth can be used to control the depth of cutting of the cranial bone 1 so as not to damage the dura 34, which is located just beneath the inner surface of the cranial bone 1. Such a drill will instantaneously stop rotating upon bone penetration (e.g. the inner table 16 of the cranial bone 1) before the drill tip reaches the dura 34. Also, the safety of creating the guiding macroaperture 3 and microaperture 4 may be increased by incrementally cutting through the cranial bone 1 (e.g. in increments of about 1 mm depth). Further, for safety, the creation of the macroaperture 3 and microaperture 4 may include a human guiding a final stage of the creation.

It may be desirable to vary the cutting during creation of the macroaperture 3 and microaperture 4, depending on their anatomical location. This may be accomplished using a cutting device which is programmed to automatically vary its operation by either slowing its longitudinal travel or slowing its cutting speed. Further, the cutting method may include a step of measuring and indicating depth of cut using, for example, physical devices such as depth stops, visual markings on the cutting device, measurement of compositional tissue changes via conductivity changes, visual methods such as optical coherence tomography utilizing a focused laser, and/or visually detecting a change of composition (e.g. from bone to dura).

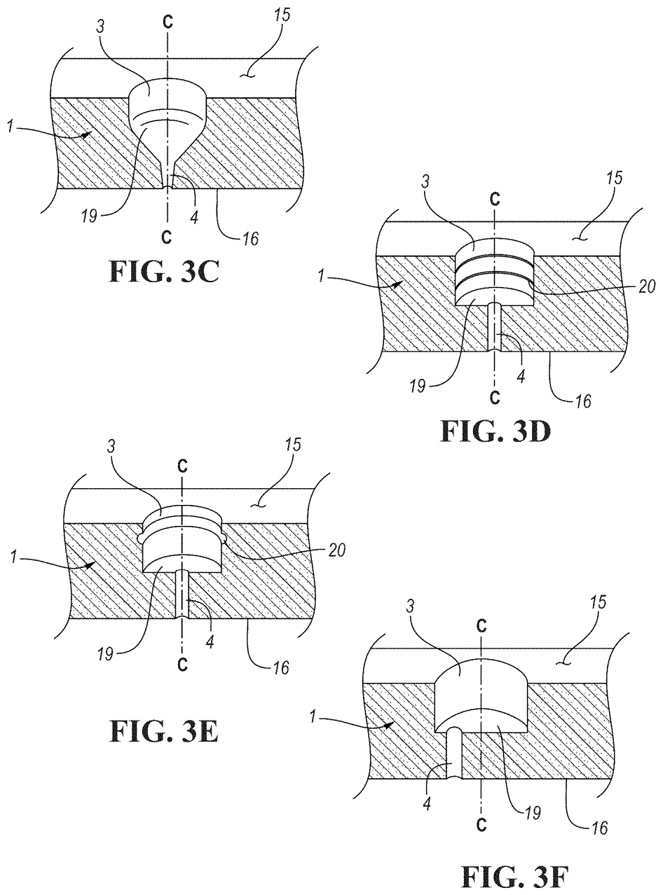

The disclosed embodiments may employ shaping at least one internal surface of the macroaperture 3 and microaperture 4 in order to define a profile which is complementary to external dimensions of the implant assembly 6. For example, FIGS. 3A-3F are cut-away side views of a portion of cranial bone 1 which illustrate several variations in shape along the longitudinal/axial direction (i.e. the direction indicated by dashed center-line C-C) of the macroaperture 3 and microaperture 4. In FIG. 3A, the macroaperture 3 and microaperture 4 are shown as substantially cylindrical and longitudinally/axially stepped to define a shoulder 19 which is the boundary between macroaperture 3 adjacent an outer surface 15 (outer table) of the cranial bone 1 and the microaperture 4 adjacent an inner surface 16 (inner table) of the cranial bone 1, which has a smaller diameter. A variation is shown in FIG. 3B, wherein the shoulder 19 becomes gradually narrower to form a frustoconical shape. FIG. 3C illustrates another variation in which the macroaperture 3 and microaperture 4 become gradually narrower (in a curve/arc) in a direction from the outer surface 15 toward the inner surface 16. By creating and positioning of the shoulder 19 in the cranial bone 1, the depth of insertion of the implant assembly 6 may be adjusted by adjusting the depth of the shoulder 19 and the length of the implant assembly 6, and the macroaperture 3 and microaperture 4 have an interior profile which limits final position and rotation of the implant assembly 6.

FIGS. 3D and 3E illustrate a macroaperture 3 formed with an engagement surface 20 which may be, for example, protruding, cupped, grooved, ridged, lined, textured, under-cut or otherwise defining a surface profile for assisting in engagement with the bone anchor 5. In FIG. 3D, the engagement surface 20 takes the form of several ridges, and in FIG. 3E the engagement surface 20 takes the form of a groove or cupping. The engagement surface 20 may form part of a mechanical interference fit with a bone anchor 5 having a complementary engagement surface on an outer surface 23 thereof, and thereby securing the bone anchor 5 in position in the cranial bone 1. The macroaperture 3 and microaperture 4 may provide a mechanical interference fit by a surface profile which includes either an undercut or a complementary surface that allows the implant assembly 6 to temporarily lock into position therein, effectively sealing the implant assembly 6 to the cranial bone 1 and preventing liquids or air from going between the cranial bone 1 and the implant assembly 6.

Considering the shape of the macroaperture 3 and microaperture 4 disclosed herein in relation to standard orthogonal X-Y-Z coordinates, FIGS. 3A-3C are longitudinally/axially symmetric. However, in some circumstances, it may be advantageous for the macroaperture 3 and microaperture 4 to be longitudinally/axially asymmetric. For example, FIG. 3F illustrates a variation of the embodiment illustrated in FIG. 3A wherein the microaperture 4 is off-center with respect to the macroaperture 3 (i.e. not along the centerline C-C). The configuration of FIG. 3F may be used when the target location of the DVS 2 is not aligned with the center of the macroaperture 3. FIG. 3F is exemplary of a variety of embodiments where the microaperture 4 and corresponding implant assembly 6 are not symmetric about a longitudinal/axial center. The variations in shape in the longitudinal/axial direction of the macroaperture 3 and microaperture 4 illustrated in FIGS. 3A-3F contribute to limiting how far the implant assembly 6 may be inserted into the macroaperture 3 and microaperture 4 and thereby prevent the implant assembly 6 from being inserted too far through the macroaperture 3 and microaperture 4 so as to guide safe insertion of the implant assembly 6.

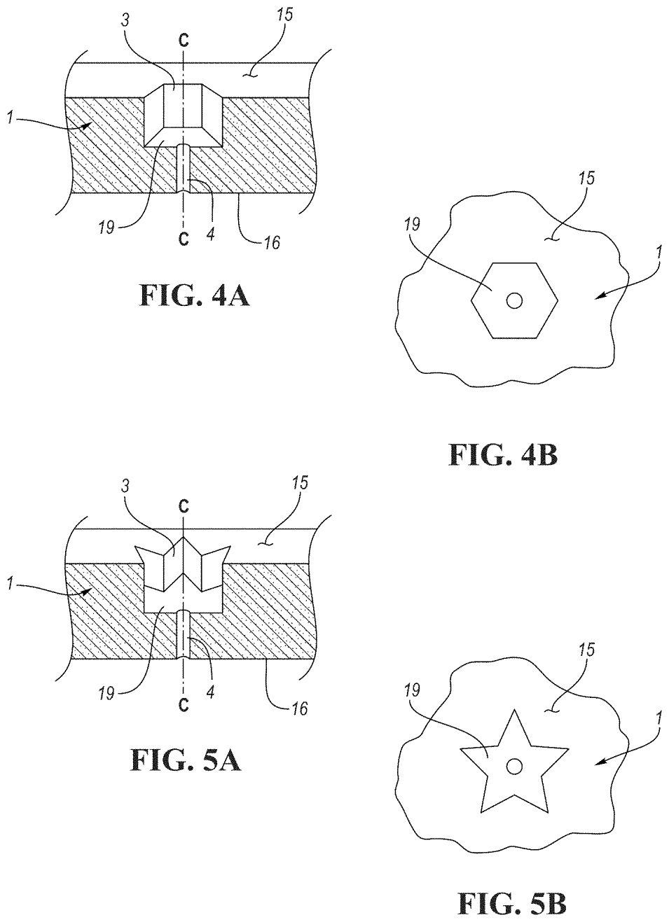

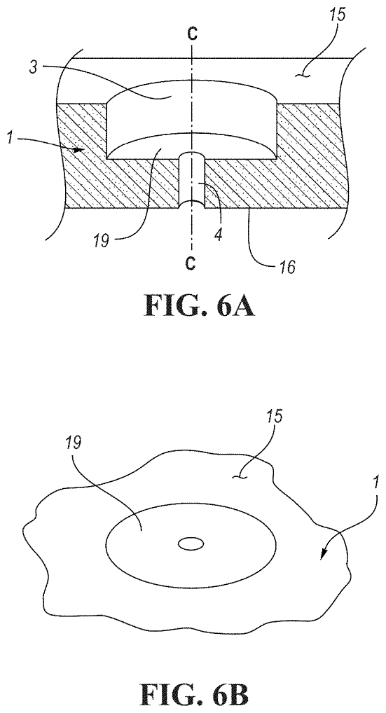

In one or more embodiments, the macroaperture 3 and microaperture 4 may have a cross-sectional profile which is substantially polygonal, circular, star, oval or crescent shaped. For example, FIGS. 4A-4B illustrate an embodiment wherein the macroaperture 3 has a hexagonal cross-sectional profile and wherein the macroaperture 3 and microaperture 4 define a hexagonal shoulder 19 therebetween. FIGS. 5A-5B illustrate an embodiment wherein the macroaperture 3 and microaperture 4 have a star-shaped cross-sectional profile and wherein the macroaperture 3 and microaperture 4 define a star-shaped shoulder 19 therebetween. FIGS. 6A-6B illustrate an embodiment wherein the microaperture 3 has an oval cross-sectional profile and wherein the macroaperture 3 and microaperture 4 define an oval-shaped shoulder 19 therebetween. As illustrated in FIGS. 4-6, the macroaperture 3 and microaperture 4 may have a cross-sectional profile which is substantially polygonal (e.g. regular, irregular, symmetric, asymmetric, triangular, rectangular, or trapezoidal), circular, star, oval or crescent-shaped, and thereby the macroaperture 3 and microaperture 4 may be configured to intimately fit together with an implant assembly 6 of a complementary shape.

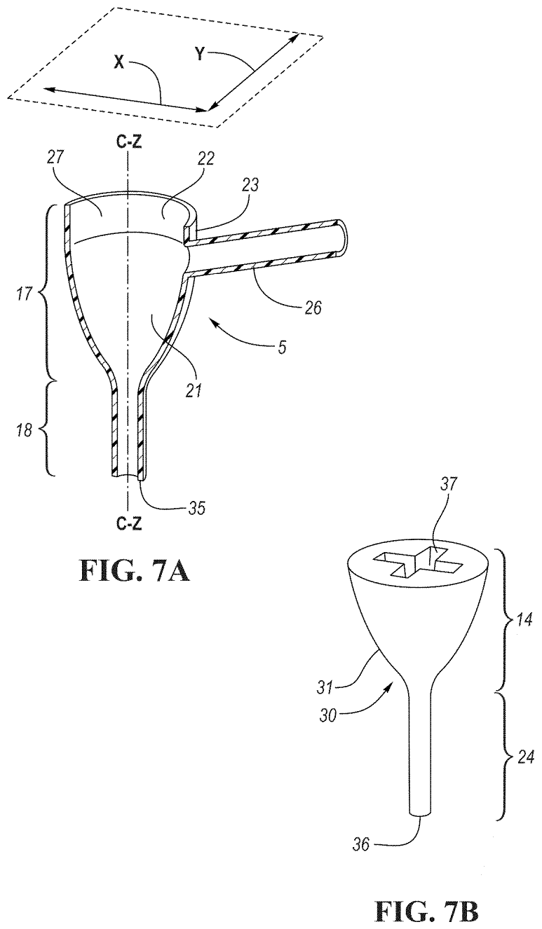

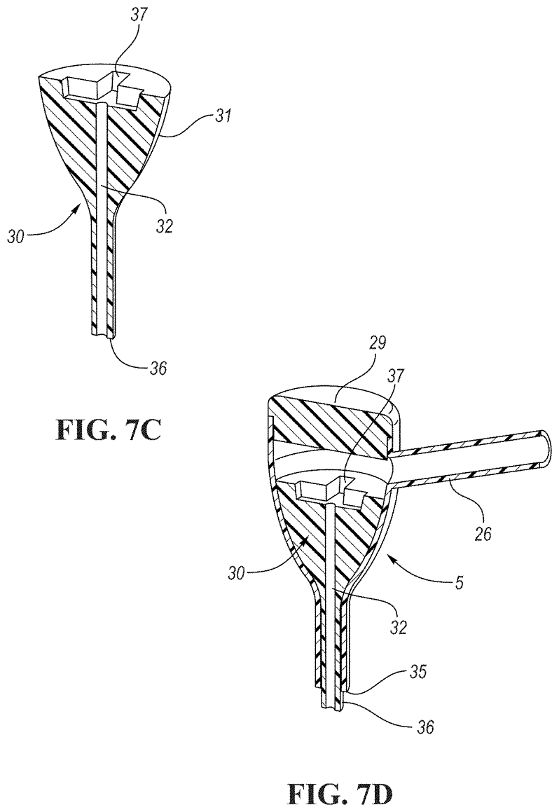

The implant assembly 6 may be a variety of different shapes to suit various different circumstances and requirements. The implant assembly 6 will be described in relation to standard orthogonal X-Y-Z coordinates, as depicted in FIG. 7A. Just as described above in regard to the macroaperture 3 and microaperture 4 shapes, the implant assembly 6 may have an outer surface that is generally longitudinally/axially symmetric or asymmetric with a cross-sectional profile which is either constant or variable along the length of the implant assembly 6.

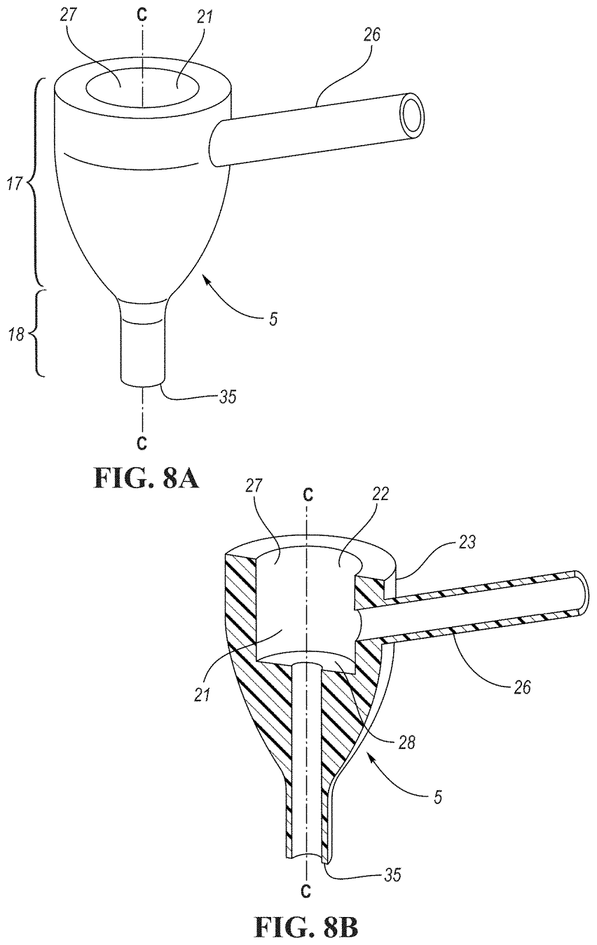

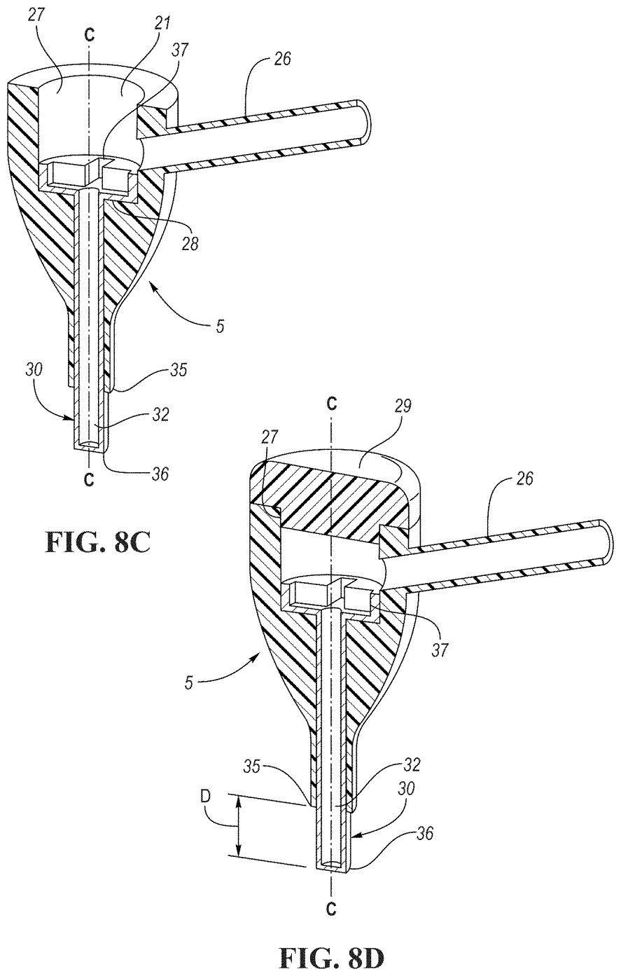

Embodiments disclosed herein contemplate the use of an implant body 30 configured to be received within and sealingly held within the bone anchor 5. FIGS. 7 and 8 illustrate bone anchors 5 having a substantially circular cross-section (i.e. in the X-Y plane, around a centerline C-C which is coincident with the Z-axis) with longitudinal/axial narrowing, and defining a lumen 21 extending therethrough. As shown in FIG. 7A, the inner surface 22 and the outer surface 23 of the bone anchor 5 have a constant offset from each other, by which the inner surface 22 and the outer surface 23 have substantially the same shape so that the bone anchor 5 has a substantially constant wall thickness. In one or more embodiments, the bone anchor 5 has an elongated configuration with an upper portion 17 and a lower portion 18, wherein the upper portion 17 has a diameter larger than a diameter of the lower portion 18 and may generally taper from the upper portion 17 to the lower portion 18. Correspondingly, the implant body 30 has an upper portion 14 and a lower portion 24, wherein the upper portion 14 has a diameter larger than a diameter of the lower portion 24 and may generally taper from the upper portion 14 to the lower portion 24. The implant assembly 6 is sufficiently long such that when placed in a combined macroaperture 3 and microaperture 4, the distal tip 36 of the implant body 30 received in the bone anchor 5 will penetrate the dura 34. FIGS. 7B and 7C illustrate an implant body 30 which has an outer surface 31 shaped to fluid-tight, sealingly fit within (i.e. have a complementary shape to) an inner surface 22 of the bone anchor 5.

The bone anchor 5 of FIGS. 8A and 8B has an inner surface 22 and an outer surface 23 which do not have a constant “offset” (i.e. “non-offset”) from each other, and therefore the inner surface 22 and the outer surface 23 do not have the same shape. Rather, the inner surface 22 includes a shelf 28. In addition, the bone anchor 5 is “short”, meaning that a length of the bone anchor 5 is such that when placed in a combined macroaperture 3 and microaperture 4, the distal end 35 of the bone anchor 5 will not penetrate the dura 34. FIGS. 8C and 8D illustrate the implant body 30 (i.e. upper portion 14) seated on the shelf 28 within the bone anchor 5, and a distal tip 36 (i.e. of the lower portion 24) of the implant body 30 extends beyond a distal end 35 of the bone anchor 5 by a distance D. The implant body 30 defines a lumen 32 extending therethrough, and has a top portion 37 configured for facilitating insertion, positioning and removal of the implant body 30. While in FIGS. 7B-7D, 8C and 8D the top portion 37 is illustrated as Phillips-head cross slots, it is understood that other structures which permit insertion, positioning, and removal of the implant body 30 may also be employed such as, but not limited to, threaded engagement regions or negative draft regions (slot or hole where the inside is larger than outside).

Both FIGS. 7 and 8 also exemplify embodiments in which the bone anchor 5 includes a conduit 26 which may be used to conduct fluid into or out of the implant assembly 6. In one or more embodiments, the conduit 26 may be integrally formed with the bone anchor 5. FIGS. 7 and 8 also exemplify embodiments in which the bone anchor 5 includes a proximal opening 27 which provides access to an interior of the implant assembly 6. As illustrated in FIGS. 7D and 8D, a resealable membrane 29 may be provided to cover the opening 27 of the bone anchor 5 (thereby covering the implant assembly 6 in general) to permit temporary and repeated access to the bone anchor 5 or implant body 30 (e.g. by a hollow needle or sensor). It is contemplated that the resealable membrane 29 may periodically be temporarily removed, for example, to replace the membrane 29 or to access the bone anchor 5 or implant body 30 (e.g. for cleaning, servicing, replacing an implant body 30, etc.). Thereby, cleaning or servicing of the implant assembly 6 may be performed in a manner that does not disturb placement of the implant assembly 6 in the cranial bone 1. As such, the disclosed embodiments permit the user to directly address issues, such as a blockage, without having to remove the implant assembly 6 from the cranial bone 1. A blockage or clot may be removed either by direct mechanical access (e.g. inserting a needle inside the implant assembly 6 to clear the blockage), or other methods, such as suction, flushing, or applying mechanical energy (e.g. ultrasonic vibration).

The implant assembly 6 may be composed of a material such as, but not limited to, metal, plastic, ceramic, resorbable compounds, biologic tissue, bone substitutes, or material derived from a cadaver. Also, the implant assembly 6 may advantageously be coated with low friction antithrombogenic material. Such a coating reduces the probability of blood clotting and potential clogging of the implant assembly 6, and reduces friction in order to facilitate insertion and removal of the implant assembly 6 into and out of the macroaperture 3 and microaperture 4. The implant assembly 6 may be coated with a material, typically a natural or synthetic polymer, which limits bony or other tissue ingrowth in order to facilitate later removal of the implant assembly 6, if needed. Examples of such materials which may be used include those in Park et al, Biomaterials to Prevent Post-Operative Adhesion, Materials (Basel). 2020 July; 13(14): 3056, incorporated by reference herein.

In one or more embodiments, the implant body 30 may be used for adjusting fluid flow resistance and thereby adjusting fluid pressure. A first implant body 30 may be inserted into a bone anchor 5, wherein the lumen 32 of the first implant body 30 defines a first fluid flow resistance. Fluid pressure adjacent (e.g. in contact with, and/or flowing into or through) the implant assembly 6 is measured, and based on that pressure measurement, the first implant body 30 is removed and a second implant body 30 is inserted in place thereof having a second fluid flow resistance different (greater or lesser) from the first fluid flow resistance. Such a method could be applied to the draining of CSF.

Also, the implant assembly 6 may have operably associated therewith or incorporated therein one or more of a fluid orifice size chosen for its ability to restrict, direct, shape or alter the CSF flow in the venous bloodstream and operate within an ideal (targeted) range of fluid volume flow rates and back pressure, a one-way check-valve, a one-way micropump, a two-way micropump, a medication reservoir, an electrode, or a sensor. For example, the sensor may function to sense and indicate at least one of the presence of CSF or blood within the implant assembly 6, the pressure of fluid adjacent, flow into and/or flowing out of the implant assembly 6, at least a component of the total intracranial pressure (ICP), both ICP and fluid volume flow and venous flow/pressure, aluminum levels to test for Alzheimer's disease, sodium levels, etc. For example, embodiments disclosed herein include a method in which the implant body 30 has a distal tip 36 (as illustrated in FIGS. 7D, 8D, 9C, 10E, 11D, and 12E) which includes a sensor, inserting the implant body 30 into the bone anchor 5 so that the distal tip 36 of the implant body 30 extends from the bone anchor 5, and then using the sensor to sense a condition of media in which the distal tip 36 is positioned (e.g. the sensor may be a pressure sensor inserted into blood used to measure blood pressure).

More broadly, the sensor may function to detect and indicate functioning of the implant assembly 6 and method for use thereof so that adjustments may be made or medication, therapy or additional care may be administered. In one or more embodiments, this could include taking a fluid sample, analyzing the fluid sample and indicating functioning based upon that analysis. Also, if an electrode is associated with the implant assembly 6, the electrode may function to receive electrical activity generated by a brain and/or administer deep brain stimulation (DBS) and receive electrical activity generated by the brain. In one or more embodiments, such an electrode may communicate with an external device for sending and receiving communications, and further employ a step of interpreting communications received by the external device for sending and receiving.

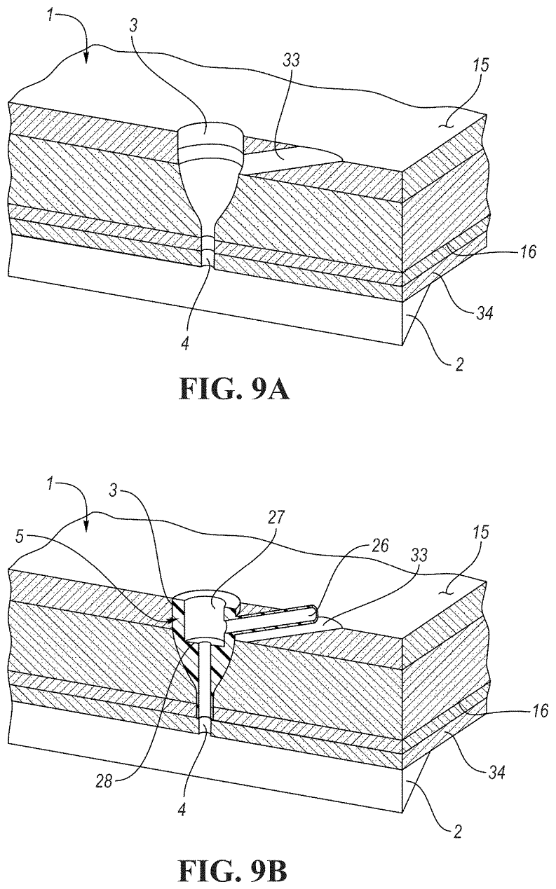

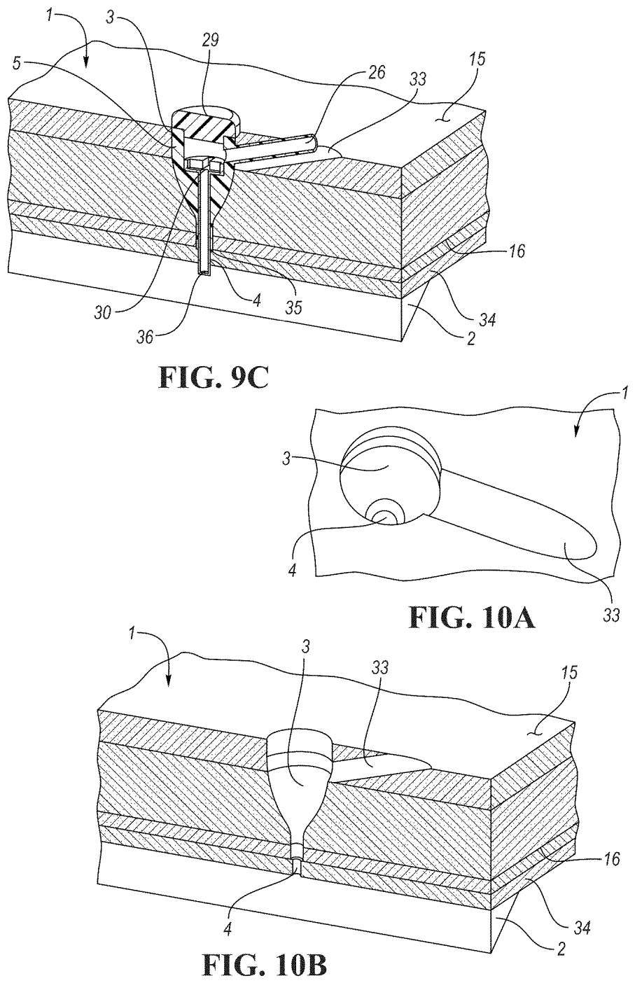

FIGS. 9A-9C, 10A-10E and 11A-11D illustrate positioning and utilization of the implant assembly 6 in relation to a cranial bone 1. FIG. 9A shows a guiding macroaperture 3 and microaperture which have been created in a section of cranial bone 1 with an outer surface 15 and an inner surface 16. In order to minimize protrusion of the implant assembly 6 from the outer surface 15, a channel 33 has been created in the outer surface 15 with one end of the channel 33 connected to the macroaperture 3. While the channel 33 is shown in FIG. 9A as angling toward the outer surface 15 (i.e. deeper where it joins the macroaperture 3, and shallower at distances further from the macroaperture 3, until the bottom of the channel 33 reaches the outer surface 15), the channel 33 may also extend substantially along or parallel to the outer surface 15 of the cranial bone 1.

While the channel 33 may be used to accommodate or contain a plurality of types of devices (e.g. a conduit, catheter, fluid transfer device, wire connection, and/or mechanical connection), it is shown in FIGS. 9B and 9C as receiving a conduit 26 of the bone anchor 5 (as previously illustrated in FIGS. 7A and 8A). The bone anchor 5 illustrated in FIGS. 9B and 9C is of the same configuration as that of FIGS. 8A-8D in that the bone anchor 5 is both “short” (i.e. the bone anchor 5 is not sufficiently long enough for the distal end 35 of the bone anchor 5 to penetrate the dura 34), and “non-offset” (i.e. the inner surface 22 and outer surface 23 do not have the same shape, but rather the inner surface 22 includes a shelf 28). FIG. 9C shows the additions of both a long implant body 30 received within the bone anchor 5 which penetrates the dura 34, and a resealable membrane 29 which sealingly covers the proximal opening 27 of the bone anchor 5.

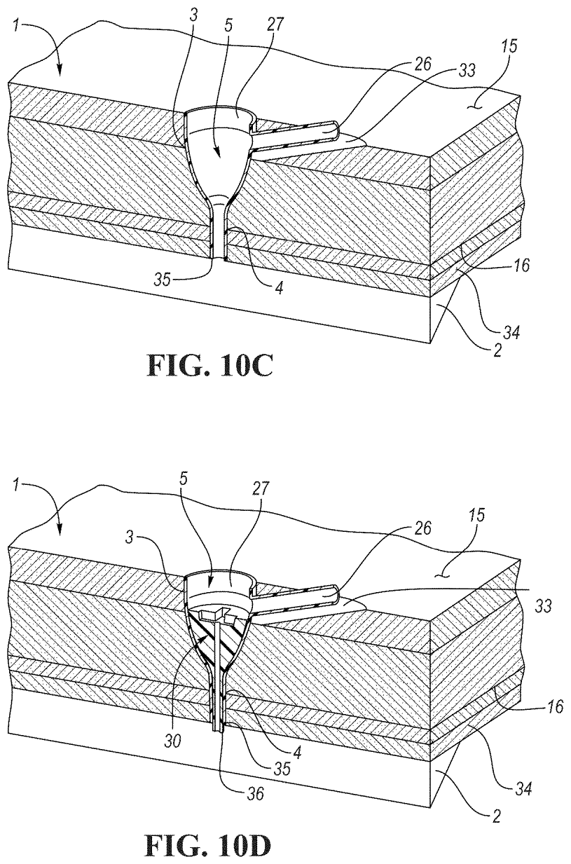

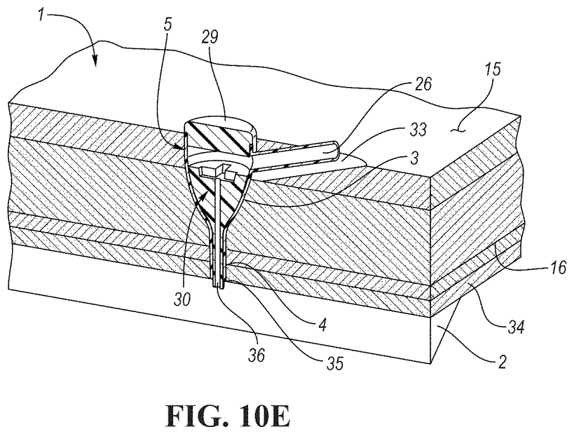

FIG. 10A is a perspective view of a section of cranial bone 1 having a combined guiding macroaperture 3 and microaperture 4 and a channel 33. FIG. 10B shows a macroaperture 3 and microaperture 4 which have been created in the section of cranial bone 1 which defines an outer surface 15 and an inner surface 16. The bone anchor 5 illustrated in FIGS. 10C-10E is of the same configuration as that of FIGS. 7A-7D in that the bone anchor 5 is both “offset” (i.e. the inner surface 22 and outer surface 23 have a constant offset from each other, by which the inner surface 22 and outer surface 23 have substantially the same shape so that the bone anchor 5 has a substantially constant wall thickness), and “long” so that when positioned in a combined macroaperture 3 and microaperture 4, the distal end 35 of the bone anchor 5 is arranged to penetrate the dura 34. FIG. 10D shows the addition of a long implant body 30 received within the bone anchor 5 which penetrates the dura 34. FIG. 10E shows the addition of a resealable membrane 29 which sealingly covers the proximal opening 27 of the bone anchor 5.

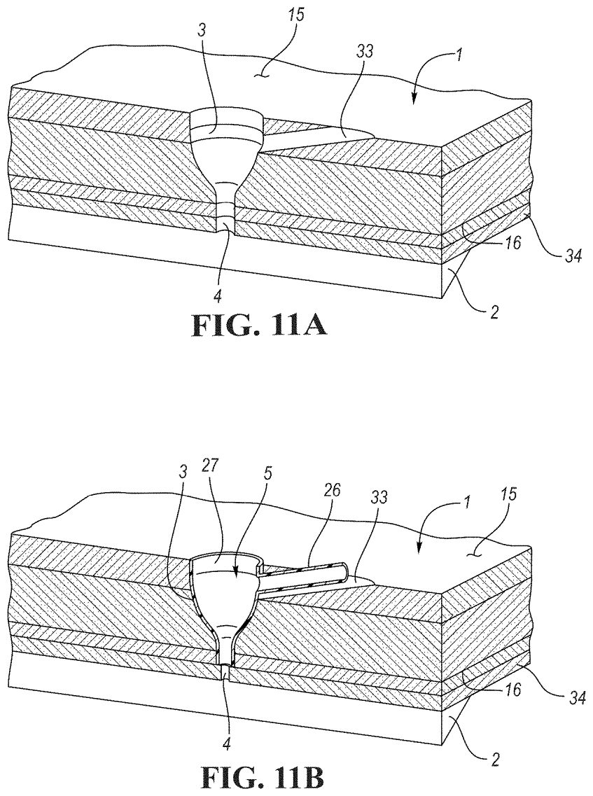

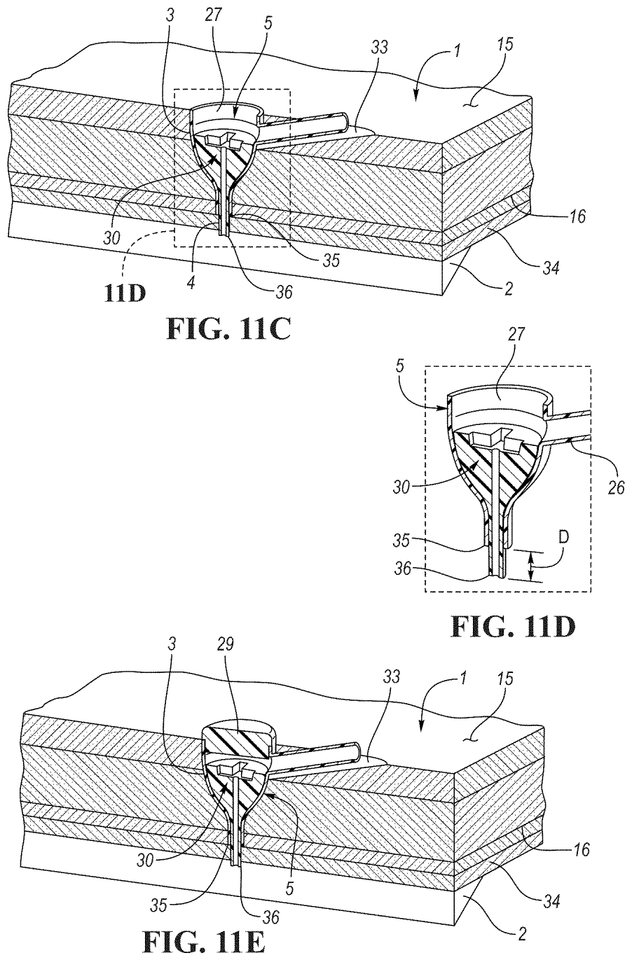

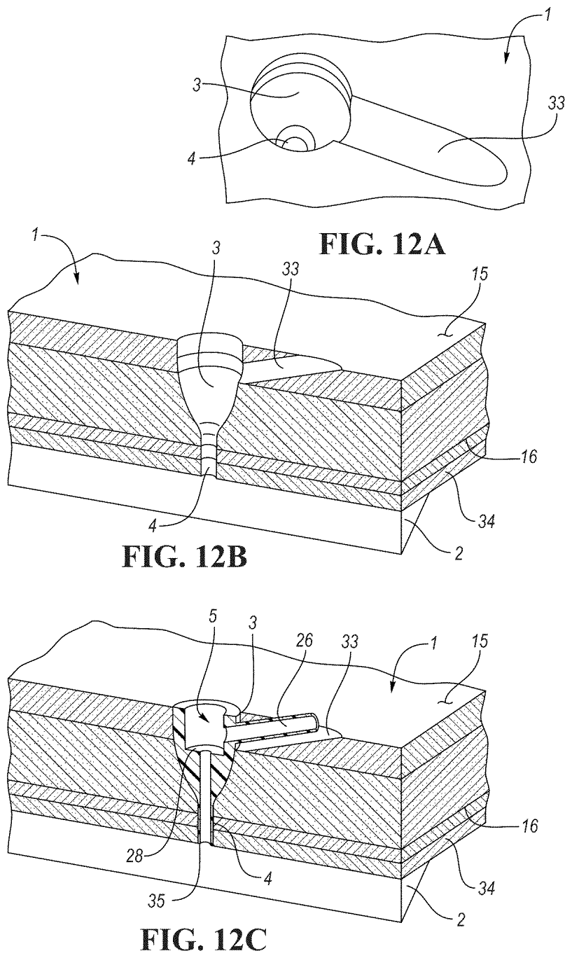

FIGS. 11A-11C and 11E are cut-away perspective views of a section of cranial bone 1. FIG. 11D is an enlarged view of the bone anchor 5 with an implant body 30 therein of FIG. 11C. FIG. 11A shows a macroaperture 3 and microaperture 4 which have been created in a section of cranial bone 1. The bone anchor 5 illustrated in FIGS. 11B-11E is both “short” (i.e. the bone anchor 5 is not sufficiently long for the distal end 35 of the bone anchor 5 to penetrate the dura 34), and; “offset” (i.e. the inner surface 22 and the outer surface 23 have a constant offset from each other, by which the inner surface 22 and outer surface 23 have substantially the same shape so that the bone anchor 5 has a substantially constant wall thickness). FIG. 11C shows the addition of a long implant body 30 received within the bone anchor 5, which is shown as extending beyond the distal end 35 of the “short” bone anchor 5 by a distance D and arranged to penetrate the dura 34. FIG. 11E shows the addition of a resealable membrane 29 which sealingly covers the proximal opening 27 of the bone anchor 5.

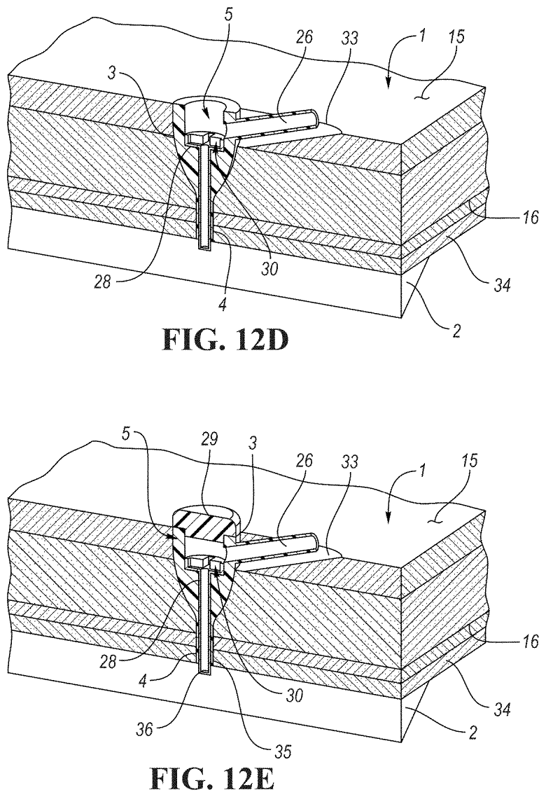

FIGS. 12A and 12B are top and perspective views, respectively, of a section of cranial bone 1 having a macroaperture 3 and microaperture 4 formed therein with a channel 33. The bone anchor 5 illustrated in FIGS. 12C-12E is both “non-offset” (i.e. the inner surface 22 and outer surface 23 do not have the same shape, but rather the inner surface 22 includes a shelf 28), and “long” so that when placed in a combined macroaperture 3 and microaperture 4, the distal end 35 of the bone anchor 5 is arranged to penetrate the dura 34. FIG. 12D shows the addition of a long cylinder implant body 30 received within the bone anchor 5 which penetrates the dura 34. FIG. 12E shows the addition of a resealable membrane 29 which sealingly covers the proximal opening 27 of the bone anchor 5.

In one or more embodiments, an exemplary method may include the steps of: 1) prior to a step of creating a guiding macroaperture 3 and microaperture 4, identifying a desired location and depth for each macroaperture 3 and microaperture 4; 2) shaping the internal surface of the macroaperture 3 and microaperture 4 to define a profile which is complementary to external dimensions of the implant assembly 6; and 3) prior to the step of inserting the implant assembly 6 into the macroaperture 3 and microaperture 4, selecting an implant assembly 6 based on shape and function from a plurality of implant assemblies 6.

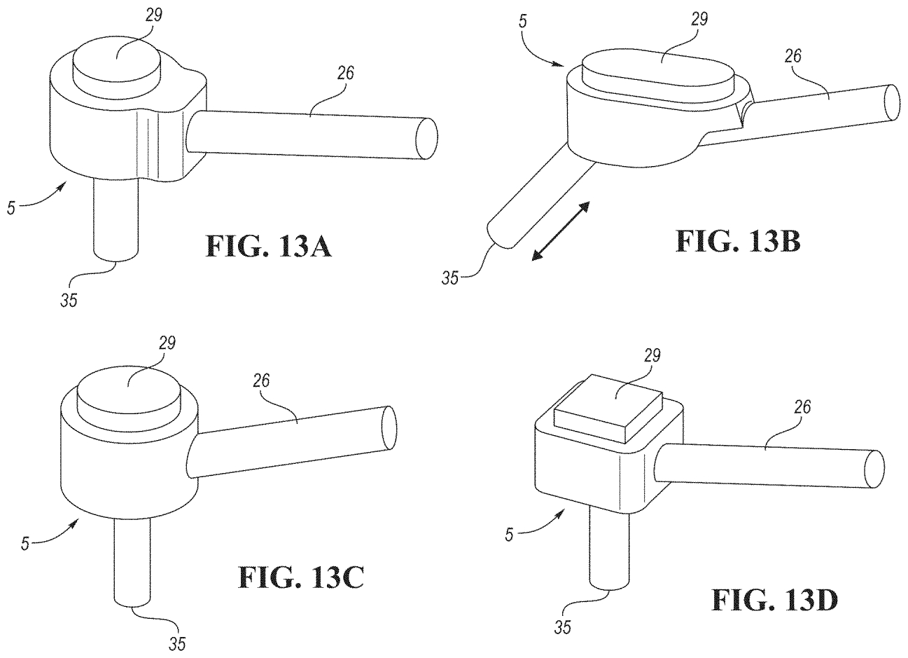

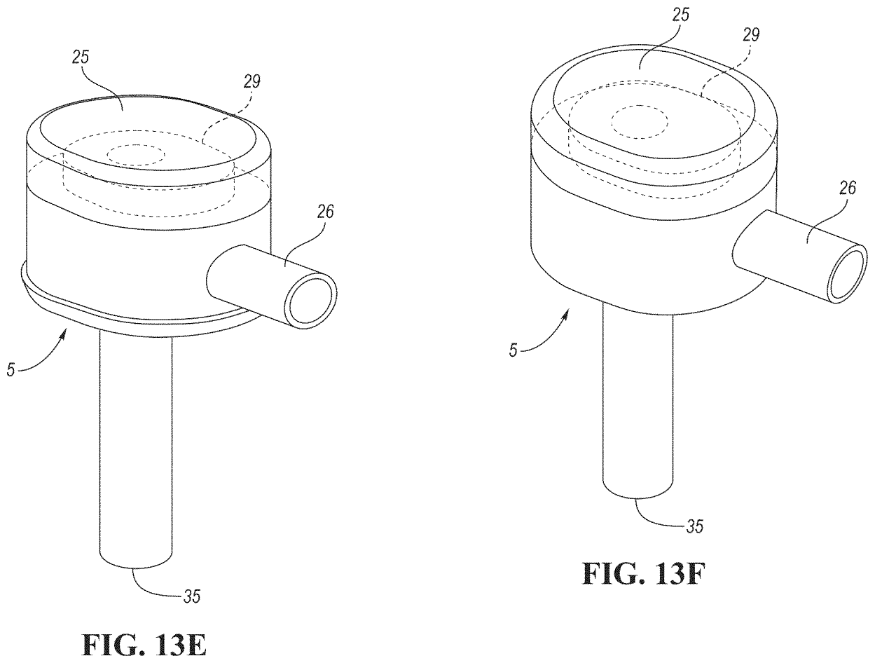

FIGS. 13A-13F illustrate various contemplated shapes and configurations of the bone anchor 5. The embodiments of FIGS. 13A and 13C-13F illustrate various configurations of a top portion 25 that provide inner space for various sensors, flow control elements and the like as well as providing positive anti-rotation and depth control by virtue of their outer shape. The configuration of FIG. 13B is advantageous in that the bone anchor 5 may be slid into a combined macroaperture 3 and microaperture 4 diagonally (i.e. in the direction of the arrow). In FIGS. 13E and 13F, the resealable membrane 29 is shown in phantom.

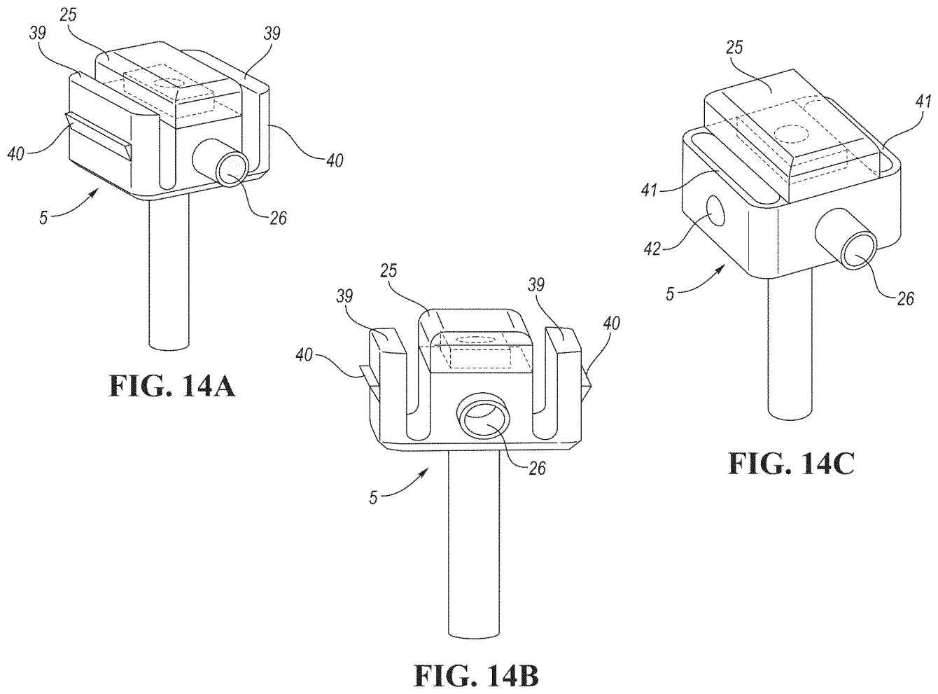

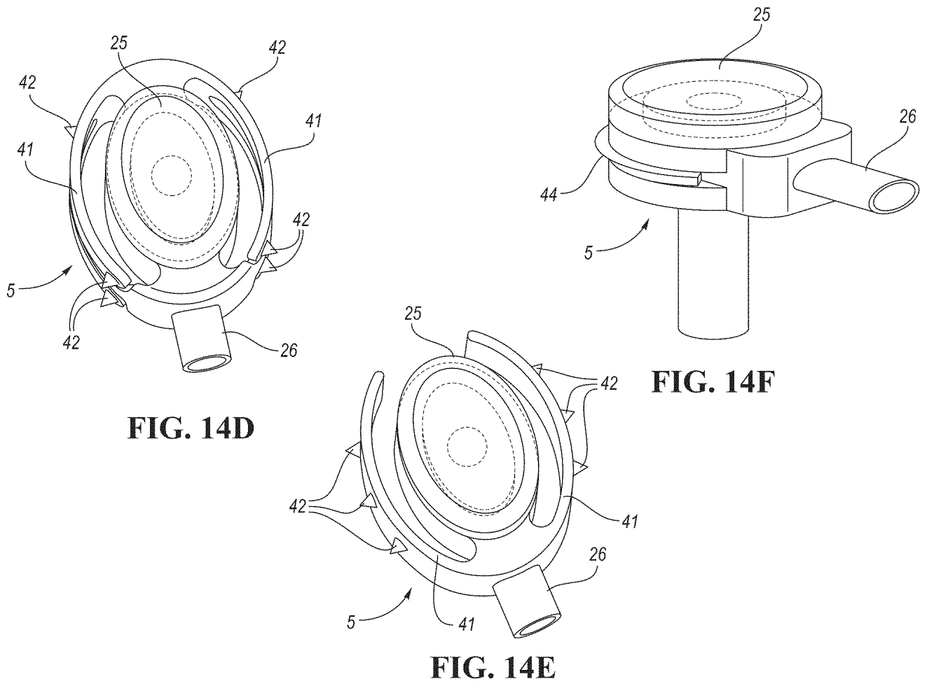

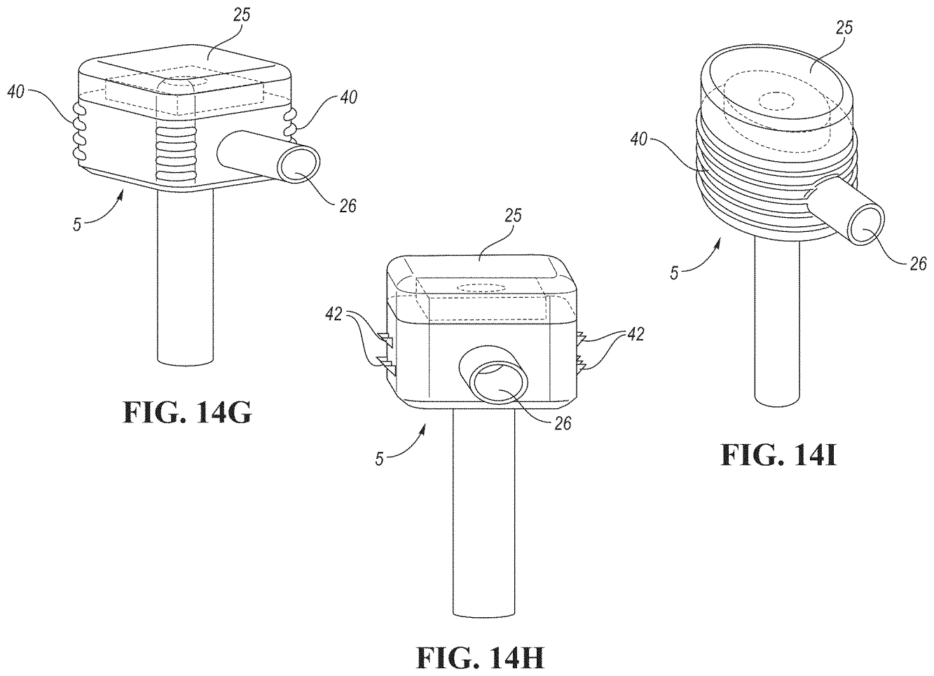

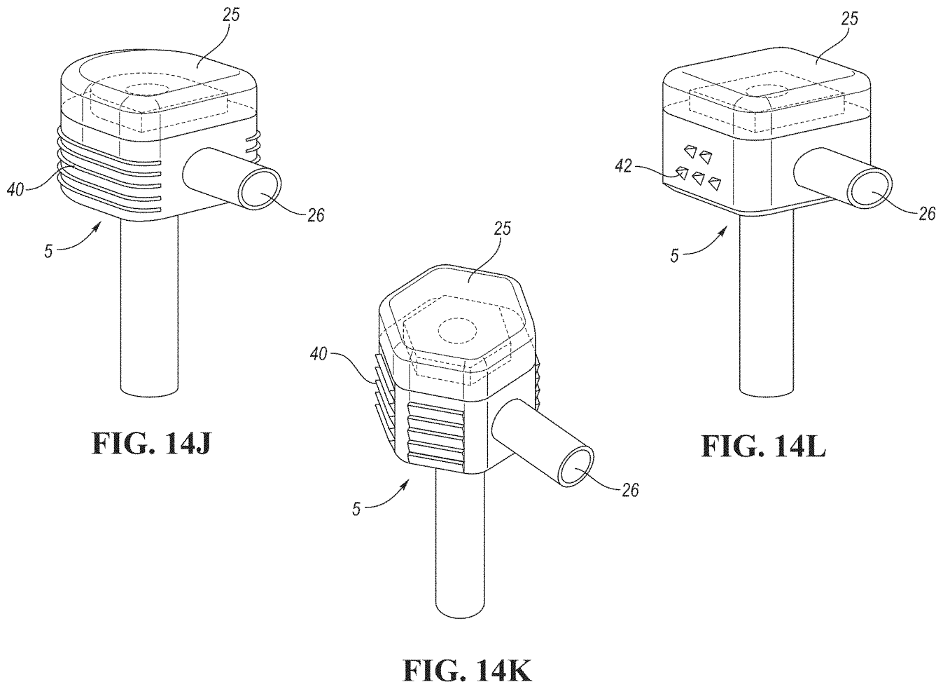

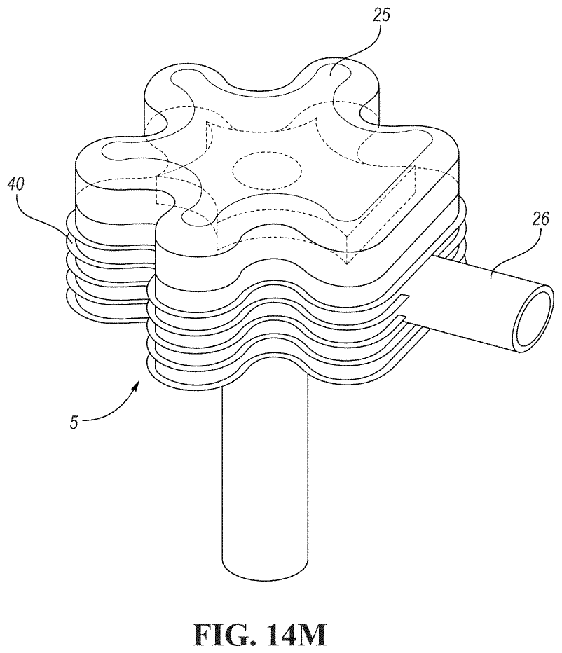

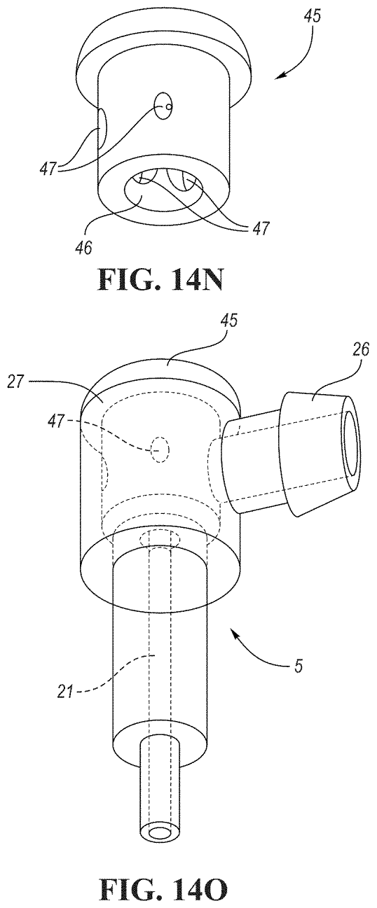

FIGS. 14A-14M illustrate embodiments in which the bone anchor 5 has an outer surface which includes at least one engaging member that is protruding (e.g. ribbed, dimpled, spikes/needles, etc.), cupped, ridged, lined, textured or otherwise defines a surface profile. FIGS. 14A-14M also illustrate various cross-sectional shapes. Such an engaging member ensures that the bone anchor 5 and the macroaperture 3 and microaperture 4 are intimately fitted together, which establishes and holds the position of the bone anchor 5 in the cranial bone 1. FIGS. 14A and 14B show two views of a configuration wherein the engaging member includes two outwardly biased flexing wings 39 on opposite sides of the bone anchor 5, with a downwardly tapering wedge-shaped rib 40 on the outer surface of each wing 39. When the bone anchor 5 is inserted into a macroaperture 3 and microaperture 4, such an engaging member provides positive spring-locking engagement with the macroaperture 3 and microaperture 4. Similarly, the configuration of FIG. 14C includes two flexing bands or walls 41 on opposite sides of the bone anchor 5 with a protrusion 42, such as a dimple, on the outer surface of each wall 41 which provides positive spring-locking engagement when inserted into a macroaperture 3 and microaperture 4.

The configurations illustrated in FIGS. 14D and 14E include two flexing bands or walls 41 on opposite sides of the bone anchor 5 with protrusions, such as spikes or needles 43, on the outer surface of each wall 41, thereby providing positive spring-locking engagement within the macroaperture 3 and microaperture 4. FIG. 14F illustrates an embodiment which includes a generally C-shaped spring 44 that is collapsed into a groove on the bone anchor 5, such as with a sleeve-like tool (e.g. analogous to a piston ring compressor tool used to install piston rings into an automobile engine). Once in place, the sleeve-like tool is removed and the spring 44 expands into an engagement surface (e.g. corresponding groove 20) in the macroaperture 3 (see FIG. 14P). FIGS. 14G-14M illustrate the use of various types and positions of ribs 40 or protrusions 42 (e.g. dimples). The engaging members illustrated in FIGS. 14A-14M are all configured to be collapsed or disengaged (e.g. with spring compression instruments) to allow the surgeon to exert positive control over the bone anchor 5 for extraction, if needed.

Another embodiment is shown in FIG. 14N which is a perspective view of a cap 45 for a bone anchor 5, and FIG. 14O which is a perspective view of the cap 45 positioned within and resealably closing a bone anchor 5. In this embodiment, the cap 45 defines a lumen 46 and a plurality of orifices 47 of various different dimensions which are in fluid communication with the lumen 46. The lumen 21 of the bone anchor 5 may be accessible through the cap 45 by use of various different structures, including having a relatively soft portion through which a sharp implement may penetrate, such as at its top (e.g. durometer measurement about 50), with the remainder of the cap 45 (i.e. its body) being relatively harder (e.g. durometer measurement about 80 to 90). Alternatively, the cap lumen 46 may extend completely through the cap 45 (i.e. open at both ends) and then be covered with a resealable membrane 29. As illustrated in FIG. 14O, when the cap 45 is inserted into the proximal opening 27 of the bone anchor 5 (i.e. into the lumen 21), the cap 45 may be rotated to select which one of the orifices 47 aligns with the conduit 26. Because the orifices 47 are of various different sizes, and hence provide different degrees of flow restriction, rotating the cap 45 allows the user to adjust restriction of fluid flowing into or out of the lumen 21, and thereby select a desired flow rate through the implant assembly 6.

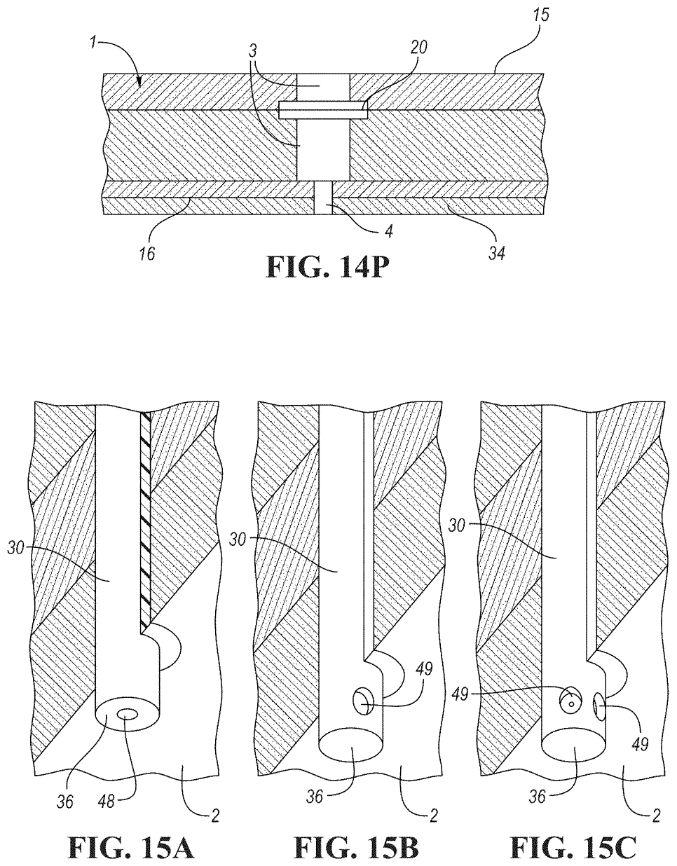

FIGS. 15A-15C are cut-away, perspective views illustrating three variations of an implant body 30 according to embodiments disclosed herein. The DVS 2 receives blood from the veins of the brain and connects with the internal jugular vein. Thus, blood in the DVS 2 flows predominantly in one direction (i.e. a first direction). It is envisioned that the lower portion 24 of the implant body 30 may be configured with one or more ports 48, 49 adjacent the distal tip 36 to protrude into the DVS 2 for directing fluid flow predominantly in a second direction. The one or more ports 48, 49 may be selectively positioned so that the first direction and the second direction are in approximately the same direction, parallel in opposite directions, or otherwise angled with respect to each other so as to improve fluid mixing, reduce probability of blockage, and reduce probability of thrombus formation. FIG. 15A illustrates an embodiment in which the distal tip 36 includes an end port 48, FIG. 15B illustrates an embodiment in which the distal tip 36 includes a side port 49, and FIG. 15C illustrates an embodiment in which the distal tip 36 includes two side ports 49. Selective positioning of the port(s) 48, 49 may be accomplished by placing or rotating the implant body 30 within the bone anchor 5 in order to select the position of the ports(s) 48, 49 relative to the direction of blood flow. Optionally, the position of the implant body 30 may be held stationary relative to the bone anchor 5 by an interference, friction or press fit, or by using one or more registering ridges and and grooves on the outer surface of the implant body 30 and the inner surface of the bone anchor 5.

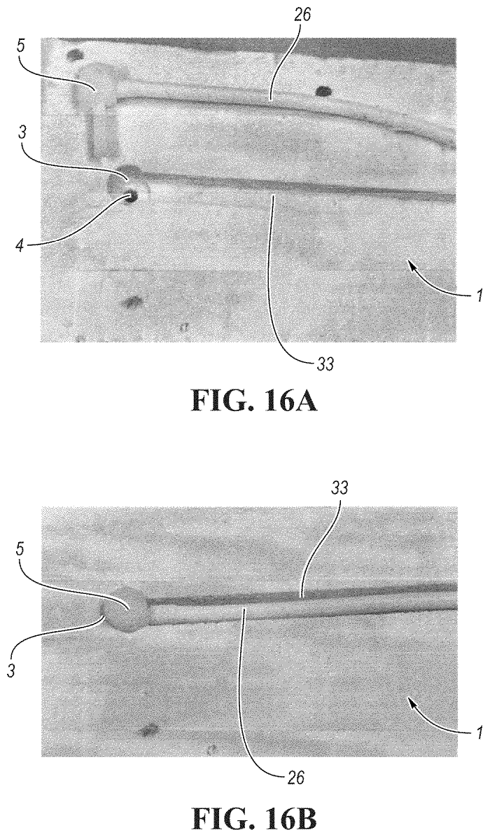

FIGS. 16A and 16B are photographs illustrating an embodiment of the implant assembly 6 and method disclosed herein. FIG. 16A shows the preparation of the cranial bone 1 to accept a bone anchor 5 with a conduit 26, wherein a channel 33, a macroaperture 3, and a microaperture 4 have been created in the cranial bone 1. FIG. 16B shows a top view of the bone anchor 5 positioned within the macroaperture 3 and microaperture 4 with the conduit 26 received within the channel 33.

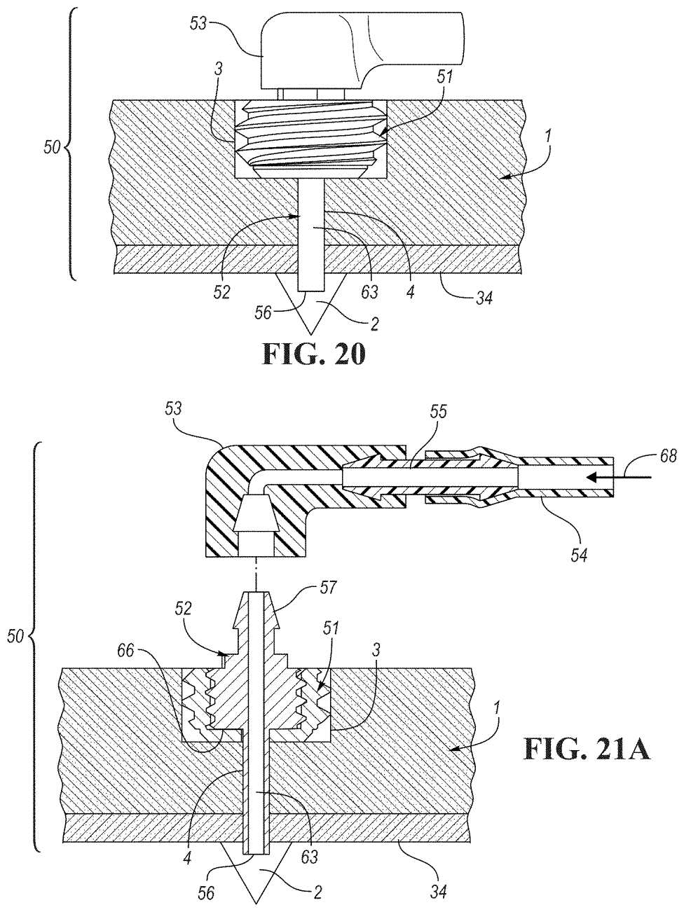

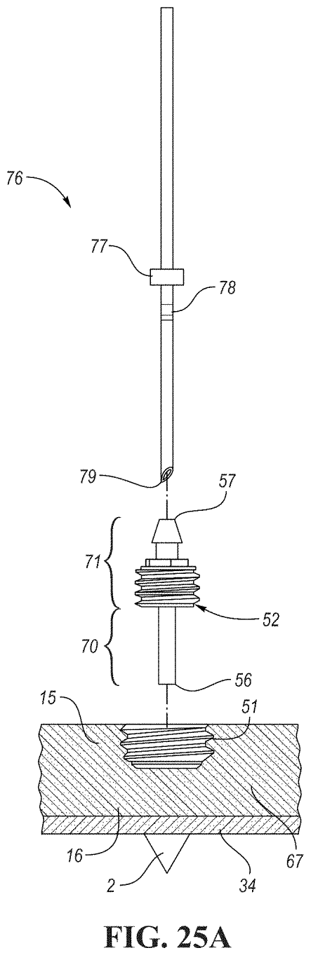

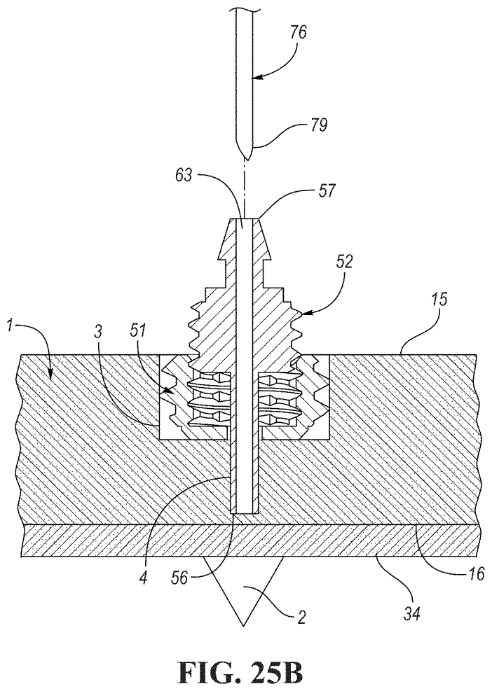

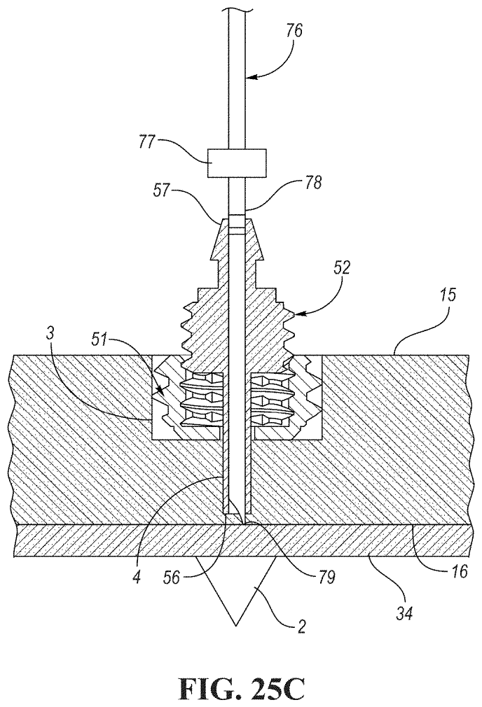

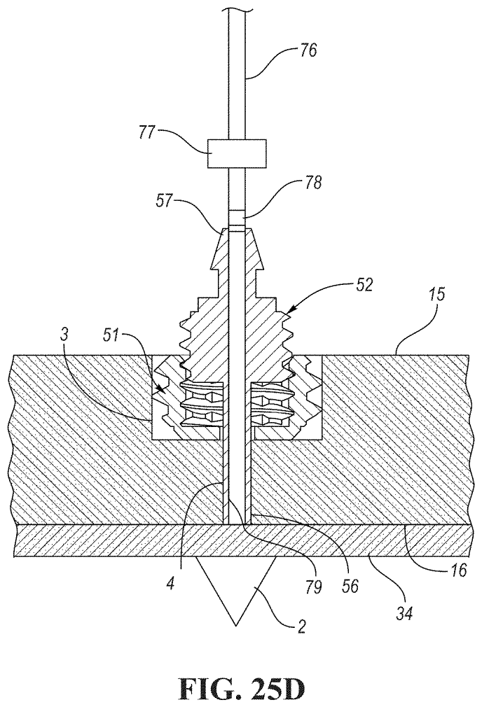

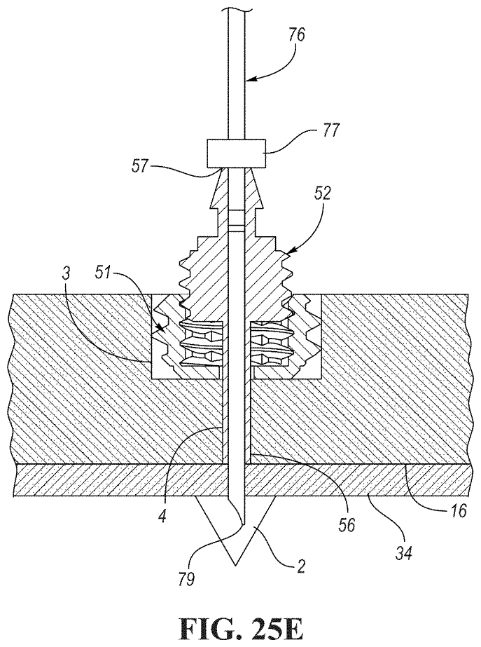

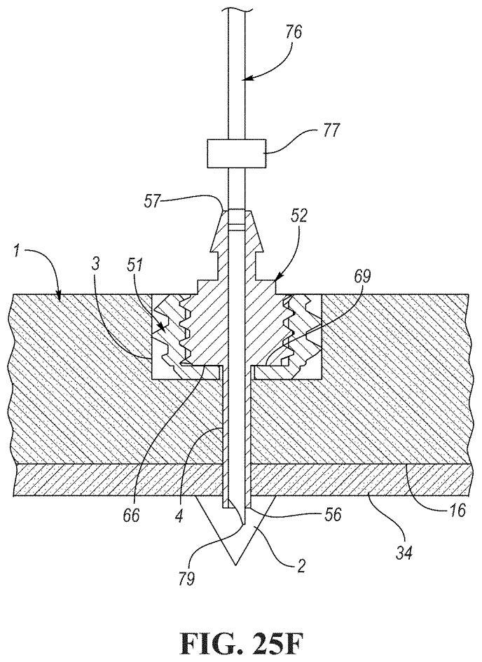

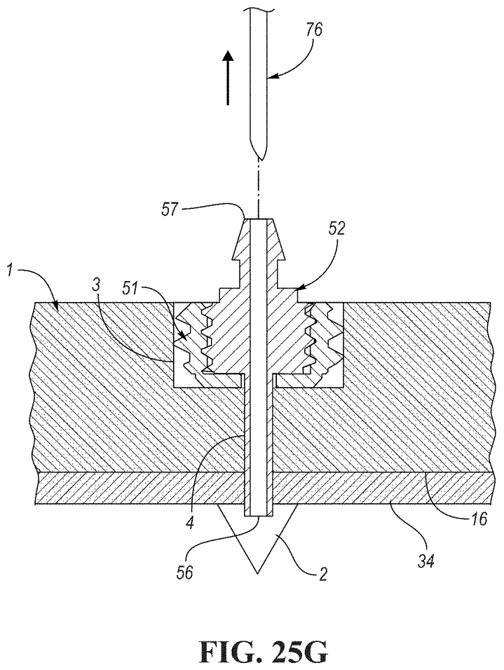

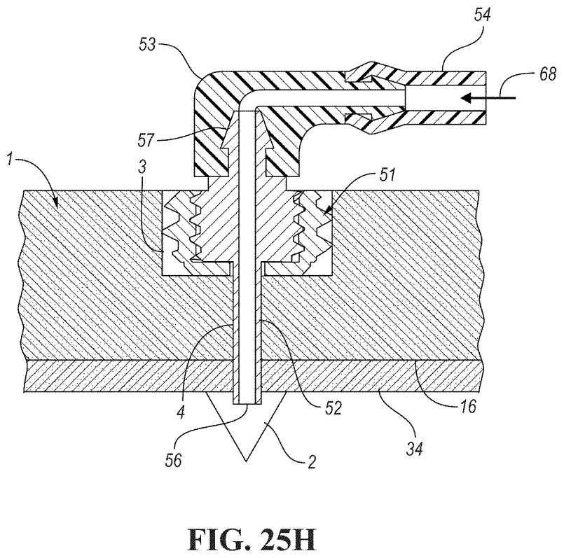

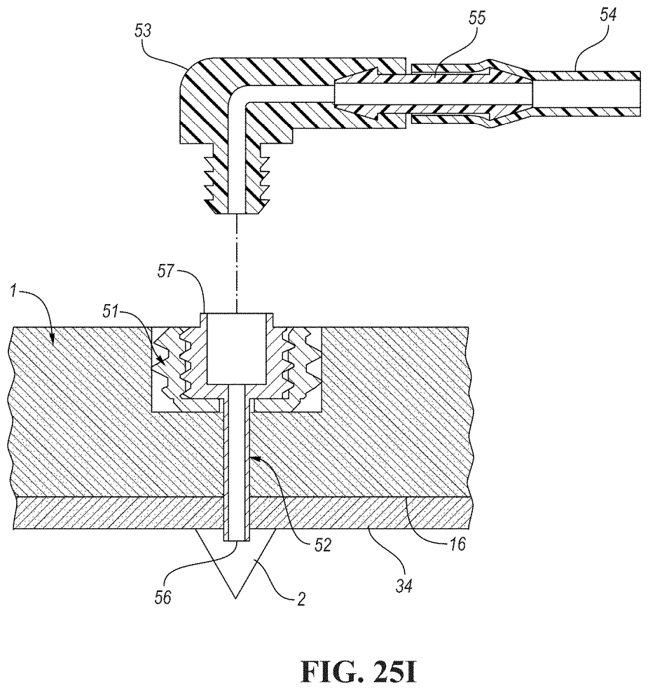

Another embodiment of an implant assembly 50 using a coaxially aligned macroaperture 3 and microaperture 4 is shown in FIGS. 20-24 and the method of implantation is shown in FIGS. 25A-29. It is understood that all features described above in connection with implant assembly 6 may be applicable to implant assembly 50, and vice versa. FIG. 20 illustrates a bone anchor 51 for secure attachment to a macroaperture 3 within the cranial bone 1. A coaxial implant body 52 with a lumen 63 extending therethrough is configured to be received within the bone anchor 51, extend through to the microaperture 4 within the cranial bone 1, and finally through the dura 34 into a fluid-containing space such as a DVS 2. In one or more embodiments, the bone anchor 51 may be about 4-12 mm outer diameter and have a thickness of approximately 3-12 mm, and be constructed from a biocompatible material such as ceramic, metal, or polymer. In one or more embodiments, the macroaperture 3 (as shown in FIGS. 20-84) may be about 4-12 mm in diameter.

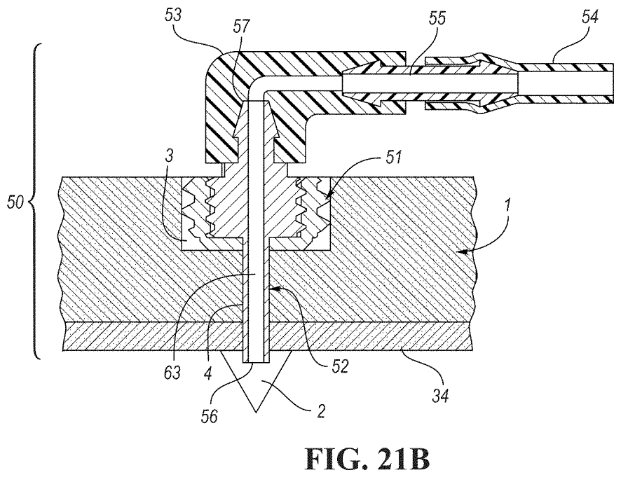

Referring to FIGS. 21A-21B, when the implant body 52 is fully received in a final position within the bone anchor 51 and a distal tip 56 of the implant body 52 is placed through the dura 34 to the desired depth (generally 0.1 mm to 2 mm), a fluid coupler 53 may be connected to a proximal end 57 of the implant body 52, which may take the form of a tapered member as shown. The fluid coupler 53 is configured to connect the implant assembly 50 to a fluid source 68 via a catheter 54 and an operably connected optional check valve or pressure valve 55 therebetween, thus establishing a fluid connection between the fluid source 68 and the DVS 2.

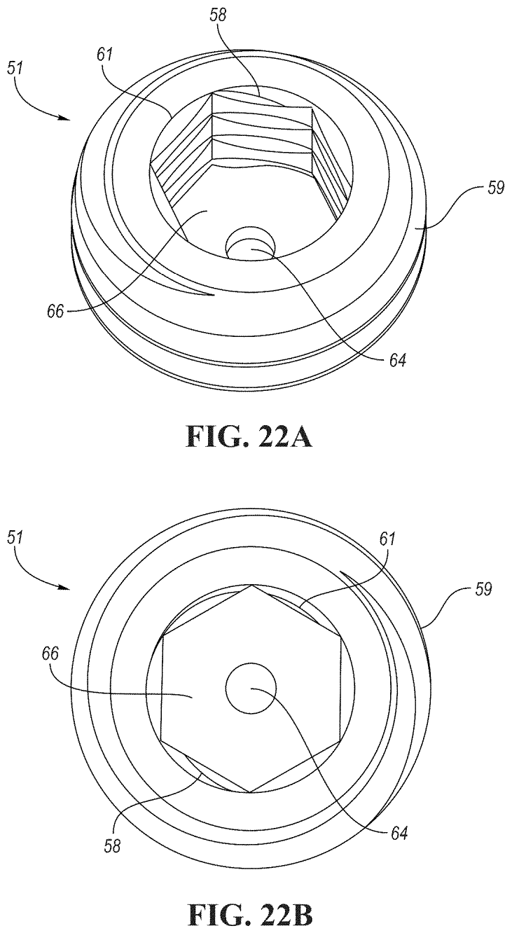

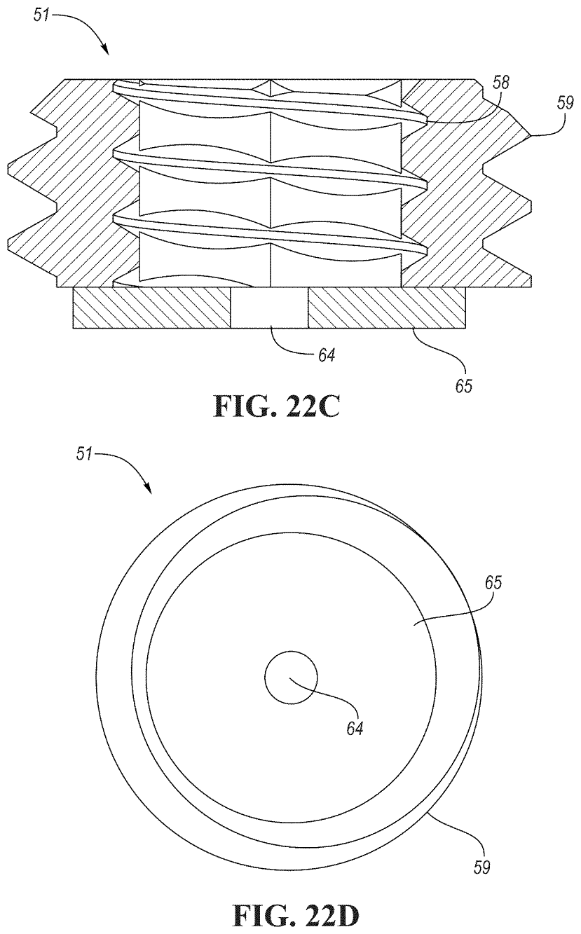

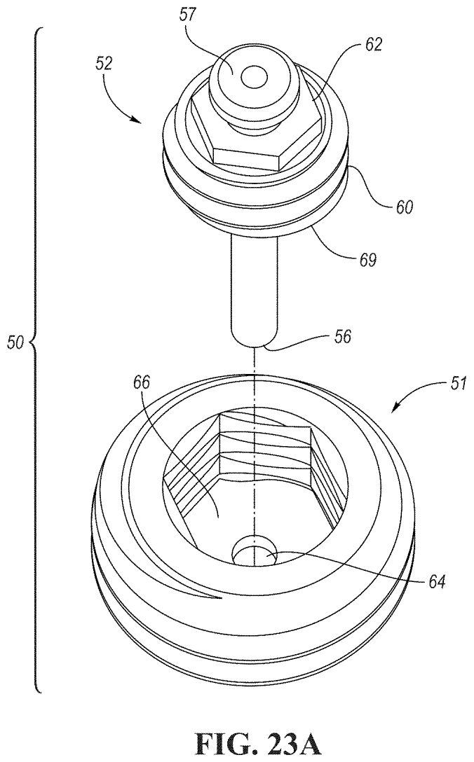

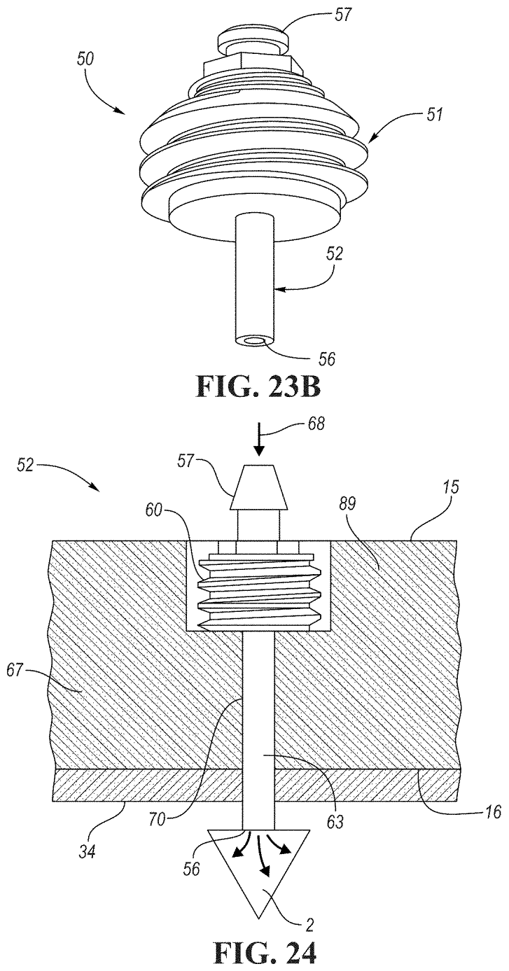

Referring to FIG. 22A, in one or more embodiments, the bone anchor 51 may be generally cylindrical and include external threads 59 for bone engagement with the macroaperture 3. The bone anchor 51 has an internal shape 61 (e.g. a hex or torx shape) which allows for tool engagement and subsequent rotation and threading into the cranial bone 1. The bone anchor 51 also includes internal threads 58 which are used to locate and secure the implant body 52 in position into the bone anchor 51. FIG. 22B shows an opening 64 in the distal portion of the bone anchor 51 which leads to the microaperture 4. Also shown is the internal shape 61 with internal threads 58 and external threads 59 as well as an internal stop surface 66 of the bone anchor 51 which engages with a distal stop surface 69 of the implant body 52 upon assembly of these components. FIG. 22C shows the bone anchor 51 in section view with internal threads 58, external threads 59, and an external stop surface 65. FIG. 22D is a bottom view the of the external stop surface 65 and opening 64 of the bone anchor 51 leading to the microaperture 4.

FIG. 23A is an exploded view of the implant assembly 50 with the implant body 52 positioned over the bone anchor 51. The implant body 52 contains a drive shape 62 for engaging an instrument (not shown) which will screw the implant body 52 into the bone anchor 51 utilizing external threads 60 on the implant body 52 to engage with the internal threads 58 of the bone anchor 51. The implant body 52 is first positioned over the opening 64 in the bone anchor 51 and then the distal tip 56 of the implant body 52 is passed through the opening 64. FIG. 23B is a perspective view of the implant assembly 50 showing the implant body 52 fully seated in the bone anchor 51.

FIG. 24 shows a side view of the implant body 52 in a final position in the cranial bone 1 with the bone anchor 51 removed for clarity. The external threaded portion 60 is shown within the macroaperture 3, generally in the area of the outer table 15 of the cranial bone 1. A cancellous region 67 of bone is found between the outer table 15 and inner table 16. A distal portion 70 of the implant body 52 is shown passing through the cancellous bone region 67, the inner table 16 portion, and finally through the dura 34. In one non-limiting embodiment, the amount of insertion depth of the distal portion 70 into the DVS 2 shown here may be between 0.1 mm to 1.5 mm. The implant body 52 may be provided in varying lengths, and the diameter of the lumen 63 may be about 1 mm+/−0.8 mm. Fluid flow, in this case CSF, enters the proximal end 57 and exits out the distal tip 56 of the implant body 52. The proximal end 57 may be configured as a connector to quickly couple to elastomeric entities such as a catheter 54 (e.g. silastic tubing) of the type typically used for ventricular shunts, or to a molded fluid coupler 53 as shown in FIG. 21A. The proximal end 57 may be configured to couple with a complementary interface of the fluid coupler 53 or can simply provide an interference fit (i.e. stretched over) with a catheter 54 of appropriate internal opening diameter to fit tightly over the proximal end 57.

FIG. 25A illustrates the implant body 52 prior to entry into the bone anchor 51. As shown, the implant body 52 has a proximal portion 71 including the external threads 60 and the proximal end 57, and an elongated distal portion 70 including the distal tip 56, wherein a diameter of the proximal portion 71 is greater than a diameter of the distal portion 70. FIG. 25A also shows additional components used to assist the surgical implantation of the implant assembly 50. To help find the proximal surface of the dura 34 and to assist in creating an opening in the dura 34 for passage of the implant body 52 into the DVS 2 or subarachnoid space, a hollow needle 76 may be utilized that has a sharp distal tip 79 and a stop member 77 with a distance between them which may be about 0.5-1.0 mm longer than a total length of the implant body 52. Such a needle 76 and implant body 52 may form a kit of the same implant size. For example, a “Size 3” kit may have a total length of which 3 mm accounts for outer table cortical bone 15, 1.5 mm of cancellous bone 67 and inner table bone 16, 0.7 mm of dura thickness 34 and a 0.7 mm DVS 2 penetration length for a total implant length of 5.9 mm. The associated “Size 3” hollow needle 76 may then have a length of 6.9 mm between the stop member 77 and the distal tip 79 to allow for minimal protrusion of the needle distal tip 79 beyond the distal tip 56 of the implant body 52. Marks 78 on the hollow needle 76 may be used to assist the surgeon determine the depth of the needle 76 relative to the implant body 52. For maximum patient safety, the stop member 77 may be utilized to limit the depth of the needle 76 passage into the DVS 2 or subarachnoid space to prevent excessive (e.g. too deep) insertion of the needle 76 through the dura 34. In one or more embodiments, the needle distal tip 79 should not reach more than 1.5 mm beyond the distal tip 56 of the implant body 52.

FIG. 25B illustrates the initial steps of a surgical procedure according to one or more embodiments once the target has been identified on the cranial bone 1, in this case, the DVS 2. A navigation system (not shown) may be used to determine the location of the macroaperture 3 to be drilled in the cranial bone 1 and, subsequently through the midline of the superior surface of the anterior third of the superior sagittal sinus (SSS). The combination of the X,Y,Z information from the navigation system as well as the trajectory angle from each of the X,Y,Z planes determines the location of the macroaperture 3 in the outer table 15. In one non-limiting embodiment, the width of the macroaperture 3 may be about 5.5 mm with a depth between 3-4 mm. The bone anchor 51 is then inserted (e.g. screwed) tightly into the macroaperture 3. A microaperture 4 is created by cutting or drilling through the opening 64 (e.g. 1 mm) in the bone anchor 51 down through the inner table 16 to a depth that just reaches the proximal surface of the SSS, which is made from the dura layer 34 that surrounds the brain.

Once the microaperture 4 has been created, the selected size implant body 52 is screwed into the bone anchor 51 until light resistance is felt from the remnants of the inner table 16 or the dura 34. At this time, the hollow needle 76 is passed through the implant body 52 until the distal tip 79 is felt touching the dura 34. Referring to FIGS. 25C-25D, the implant body 52 is then advanced by rotation to a position that just shows the markings 78 (e.g. yellow/green) on the hollow needle 76 at the proximal end 57 of the implant body 52. In one or more embodiments, this position represents that the distal tip 56 of the implant body 52 is now in contact with the interface of the inner table 16 and the dura 34. Now, referring to FIG. 25E, the hollow needle 76 is advanced until the stop member 77 reaches the proximal end 57 of the implant body 52. About this time, a flash of venous blood should come out of the proximal end of the needle 76. If this occurs, this means that the distal tip 79 of the needle 76 has passed through the superior surface of the DVS 2.

Now referring to FIG. 25F, the hollow needle 76 remains through the dura 34 while the implant body 52 is screwed in completely and the distal stop surface 69 is resting on the internal stop surface 66 of the bone anchor 51. This will result in the distal tip 56 of the implant body 52 protruding through the dura 34 by a predetermined distance. As shown in FIG. 25G, the needle 76 is now removed leaving the assembled bone anchor 51 and implant body 52 securely in place. FIG. 25H represents the completion of the implantation surgery by connecting the fluid coupler 53 to the proximal end 57 of the implant body 52, followed by optionally connecting the fluid coupler 53 to a check/pressure valve 55 and then to the inlet catheter 54 carrying the CSF. Once all steps are completed, CSF will flow into the DVS 2. The implantation of the implant assembly 50 is now completed and the patient can be closed. FIG. 25I illustrates an alternate construction for the fluid coupler 53 and the proximal end 57 of the implant body 52 where the male-female features are reversed.

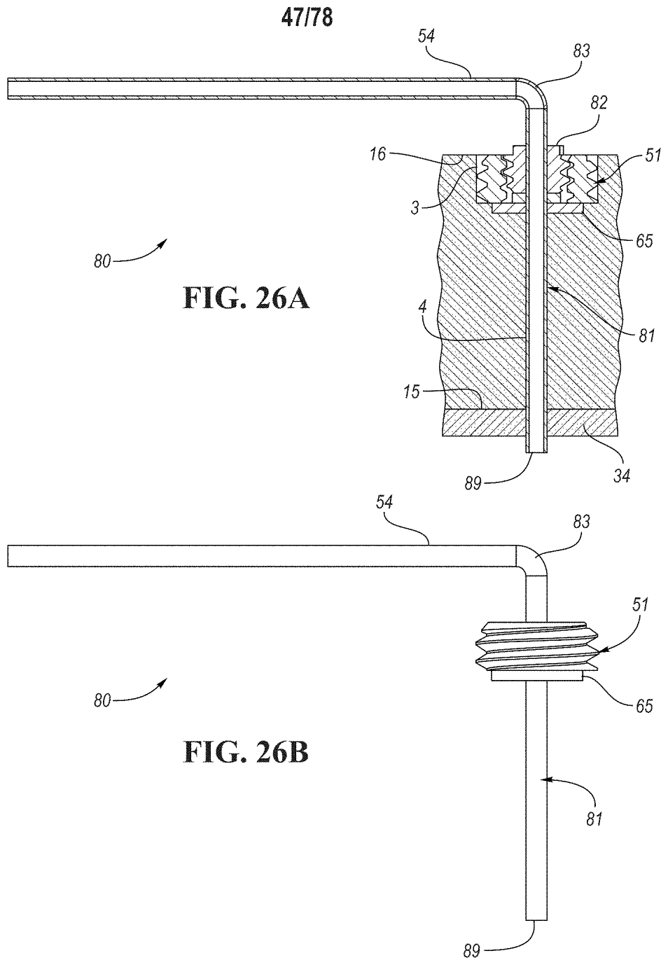

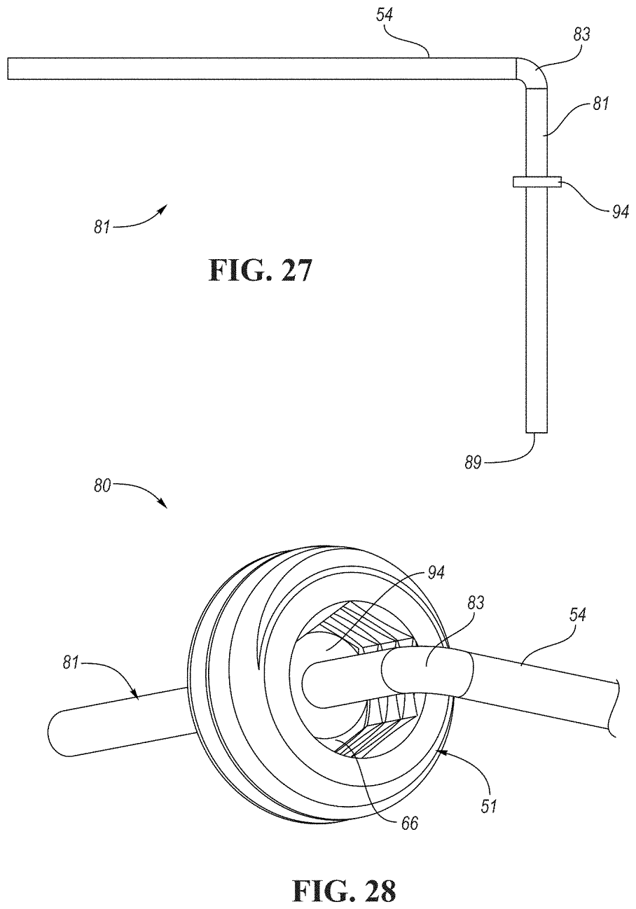

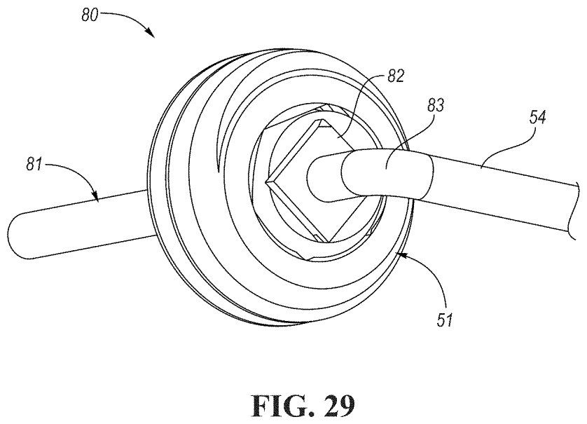

Another embodiment is shown in FIGS. 26A-29 which includes an implant assembly 80. It is understood that all features described above in connection with implant assembly 6 and implant assembly 50 may be applicable to implant assembly 80, and vice versa. FIG. 26A shows a sectional view of a bone anchor 51 as described above but which now uses a tubular implant body 81 of constant cross-sectional area with a fixed stop plate 94 (FIG. 27) that has a similar distal length as measured from the distal surface of the stop plate 94 to a distal tip 89 of the implant body 81. FIG. 26B shows a side view of an implant assembly 80 with the implant body 81 received in the bone anchor 51, whereas FIG. 27 is a side view of the implant body 81. FIG. 28 shows that after the tubular implant body 81 has been positioned into the bone anchor 51 and the microaperture 4, it will reach its maximum insertion depth when its stop plate 94 mates with the internal stop surface 66 of the bone anchor 51. If the implant body 81 is sized correctly, its distal tip 89 will have protruded through the dura 34 by the desired amount (FIG. 26A) into the target fluid-containing space (DVS 2 or subarachnoid space). FIG. 29 illustrates how the tubular implant body 81 is then locked into place to the bone anchor 51 with a locking nut 82 which has exterior threads to match the internal threads 58 found in the bone anchor 51. A coupler 83, similar in function to the coupler 53, may be used to connect with an optional check/pressure valve 55 and catheter 54.

In other embodiments, once the macroaperture 3 is established, a method is contemplated where determining the location and trajectory of the microaperture 4 does not require that the macroaperture 3 and microaperture 4 are coaxial to one another. An implant assembly 114 (including a bone anchor 100 and an indexable implant body 113) according to one or more embodiments will now be described, wherein it is understood that features described above with respect to implant assembly 6, implant assembly 50, and implant assembly 80 may be applicable to implant assembly 114, and vice versa.

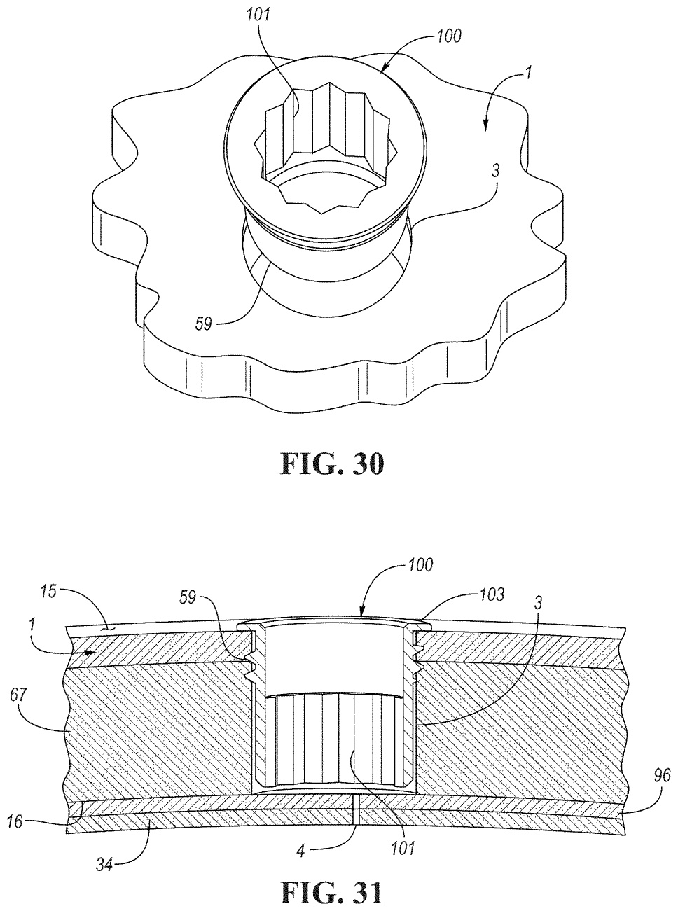

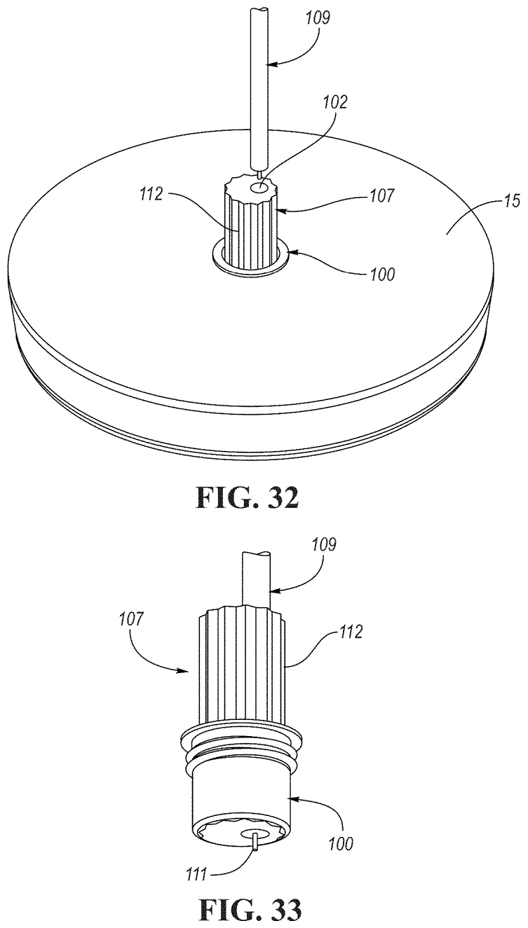

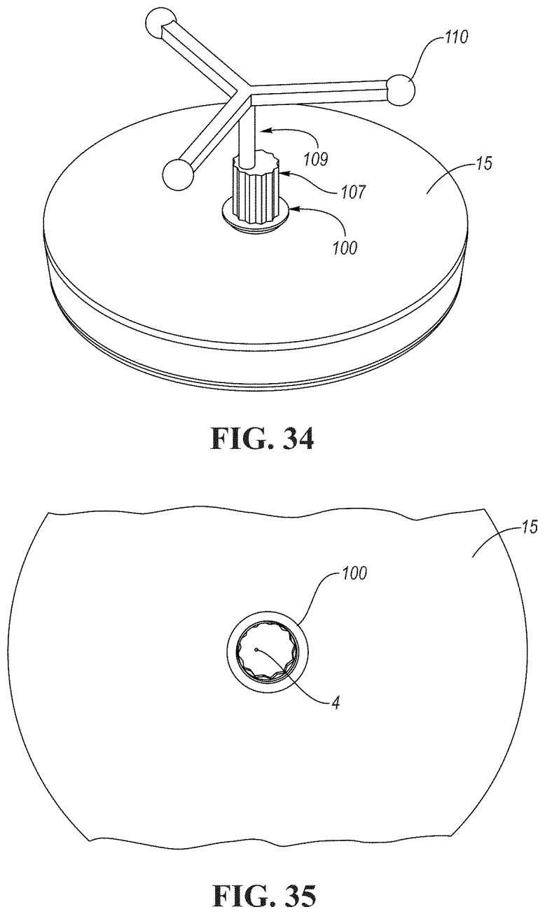

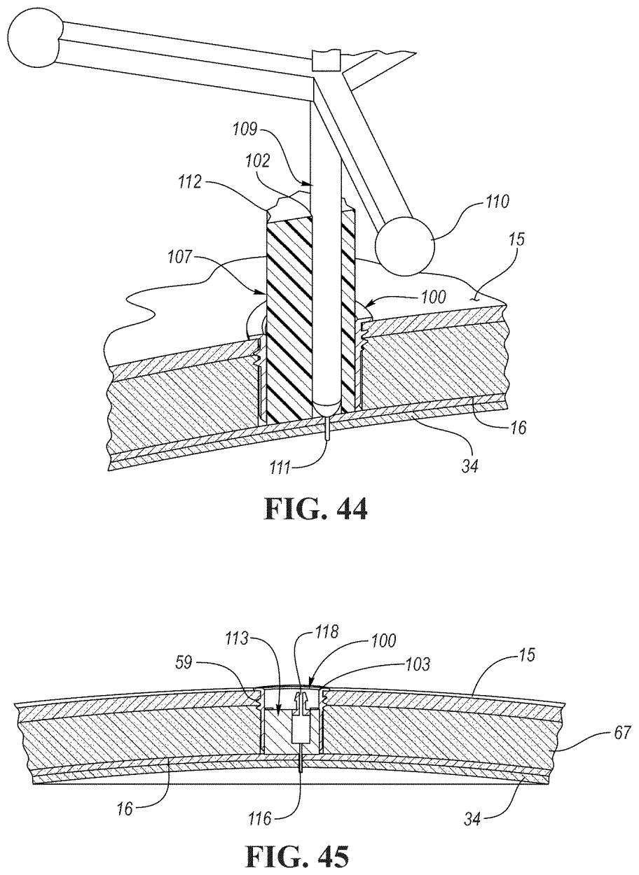

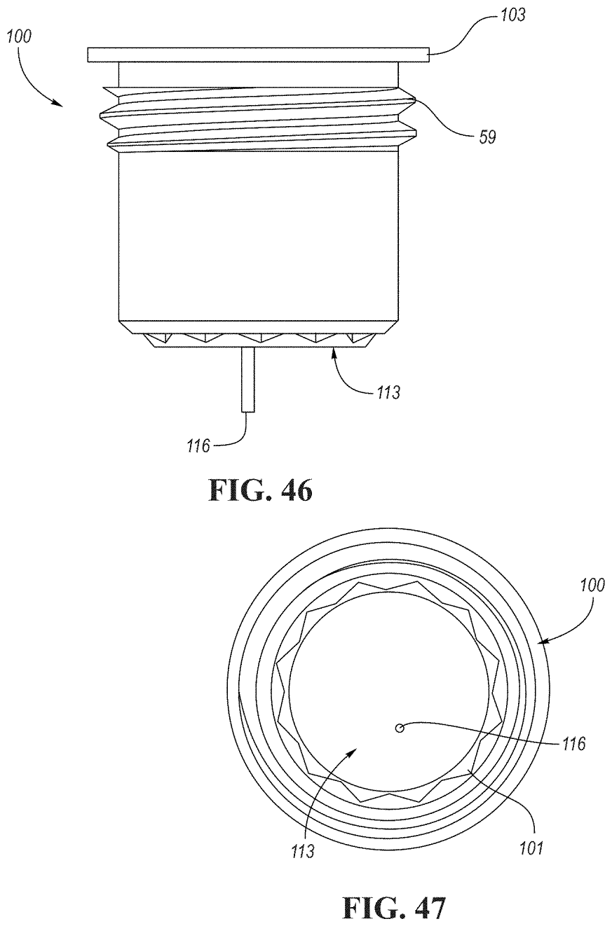

With reference now to FIGS. 30-35 and 43-44, the macroaperture 3 is shown in the cranial bone 1 with a bone anchor 100 designed to be received and intimately fit within the macroaperture 3 and locked into position with its external threads 59. The bone anchor 100 has internal facets 101 that provides engagement for a driving tool (e.g. a 12-pointed driver; not shown) to rotationally screw the bone anchor 100 into the outer table 15. The bone anchor 100 has an upper flange 103 configured to limit the depth of insertion of the bone anchor 100 in the cranial bone 1. The internal facets 101 used to engage the driving tool may extend partially (FIG. 31) or completely (FIG. 30) from the proximal to distal interior surface of the bone anchor 100. The bone anchor 100 is sized in length to fit the depth of the macroaperture 3. In some cases, the bone anchor 100 may reach the remaining inner table 16. The bone anchor 100 utilizes offset axial alignment with the microaperture 4 (e.g. 1.5 mm to 2 mm) to allow for more accurate final placement of the microaperture 4, and generally allows for the placement of the microaperture 4 anywhere inside the internal diameter of the bone anchor 100.

Once the bone anchor 100 has been placed, an indexing tool 107 is introduced into the bone anchor 100, wherein the indexing tool 107 is movable with respect to the bone anchor 100 in fixed angular steps. The external facets 112 of the indexing tool 107 are complementary to the internal facets 101 of the bone anchor 100. The indexing tool 107 has a lumen 102 extending therethrough with a longitudinal axis that is offset from the central longitudinal axis of the indexing tool 107. A probe 109, which may be connected to a CT/MRI 3D scan which utilizes reflectors/transmitters 110, or an ultrasound tip or infrared sensor to detect the DVS 2, may be passed through the lumen 102 until the probe tip 111 reaches the remaining inner table 16. The location of the DVS 2 is noted on navigation software (e.g. Stealth by Medtronic) and the probe 109 is moved into a position closest to an intended ideal location 129 on the DVS 2.

In one or more embodiments, the bone anchor 100 may include a numbering or other index marks, such as on the upper flange 103 thereof, that is viewable by the surgeon. There may also be corresponding index marks on a viewable surface (e.g. the top surface) the indexing tool 107. When the indexing tool 107 aligns with the desired location 129 on the DVS 2, the position of the index marks on the indexing tool 107 is noted relative to the index marks on the bone anchor 100, and a depth-limiting drill or other cutting device is introduced to penetrate the remaining inner table bone 16 and the superior surface of the DVS 2 at the ideal location 129. The length of the cutting device may be determined by reading the depth of the dura 34 from the outer table 15 on the patient's CT/MRI scan and deducting the length of the bone anchor 100 selected for the patient. In one non-limiting embodiment, the outer diameter of the macroaperture 3 may be about 10 mm with external dimensions of the bone anchor 100 of about 10-25 mm and internal dimensions of about 8 mm, and the diameter of the microaperture 4 of about 0.5 mm, although other dimensions are also contemplated.

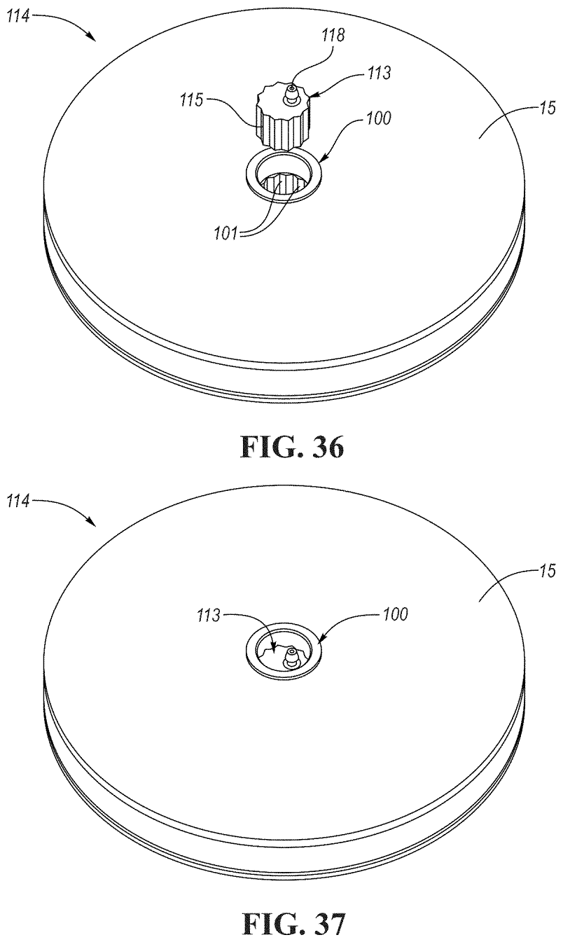

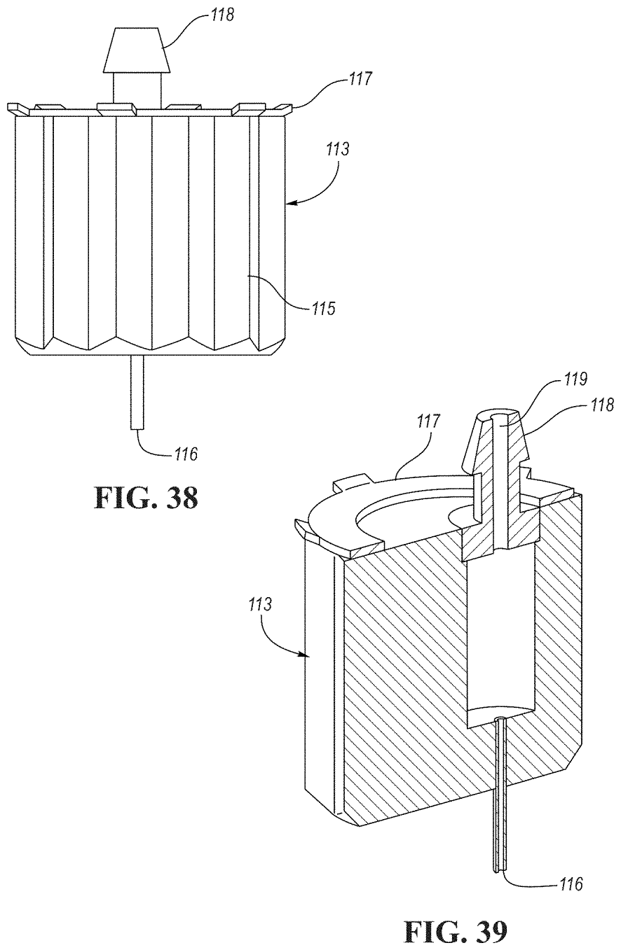

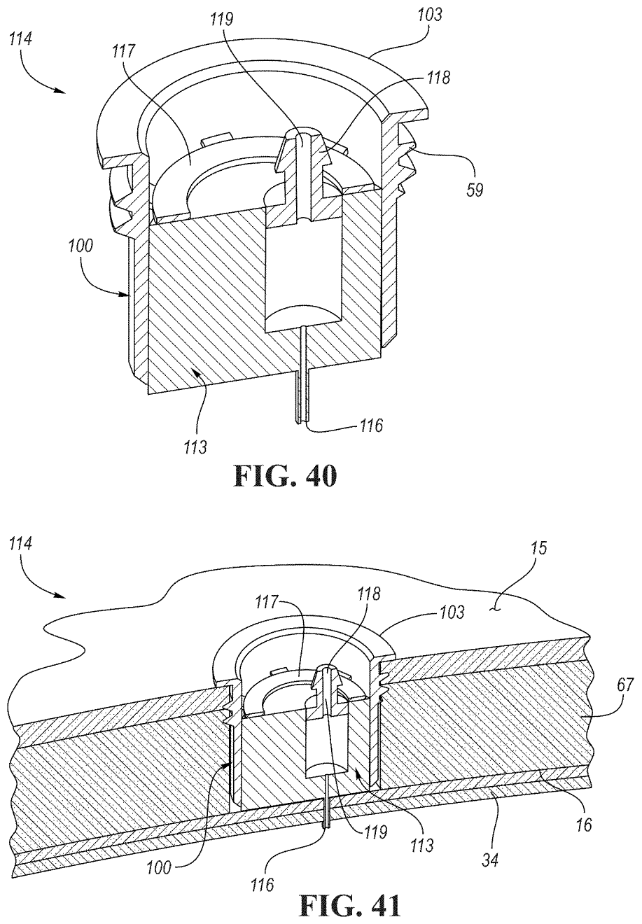

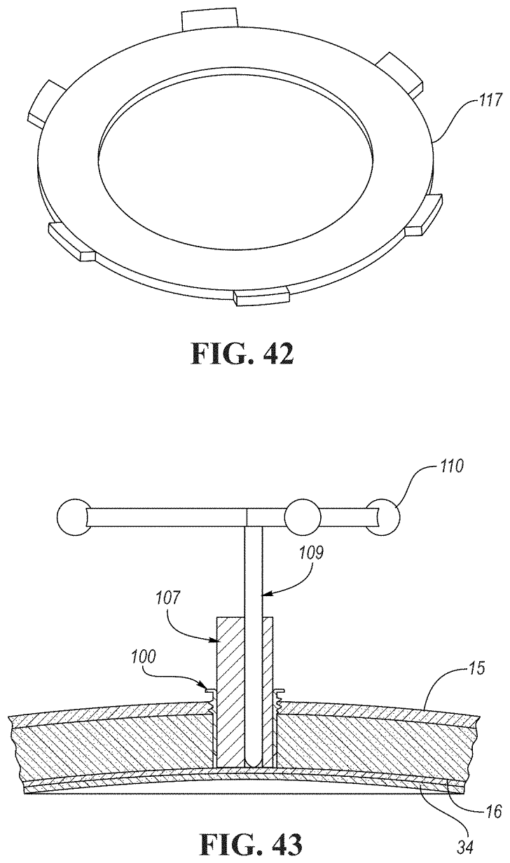

When a flash of blood occurs indicating access to the DVS 2 has happened, the indexing tool 107 is removed (FIG. 35). With reference to FIGS. 36-42 and 45, an indexable implant body 113 (also movable with respect to the bone anchor 100 in fixed angular steps) with external facets 115, a distal tip 116, and an offset lumen 119 (having a longitudinal axis offset from a central longitudinal axis of the implant body 113) of the appropriate length is introduced into the bone anchor 100 and seated on the remaining inner table 16, noting that an index mark (not shown) on the indexable implant body 113 is properly aligned with the corresponding index mark (not shown) on the bone anchor upper flange 103 as determined in the previous navigation step. The indexable implant body 113 is then seated and locked into place to the bone anchor 100 using a locking ring 117, such as a spring washer or equivalent securing structure. The elastomeric catheter 54 (e.g. from the lateral ventricle of the patient is then joined to the implant body via connector 118. At this point, a membrane 29 can then be placed over the proximal surface of the implant assembly 114 and the surgical site closed.

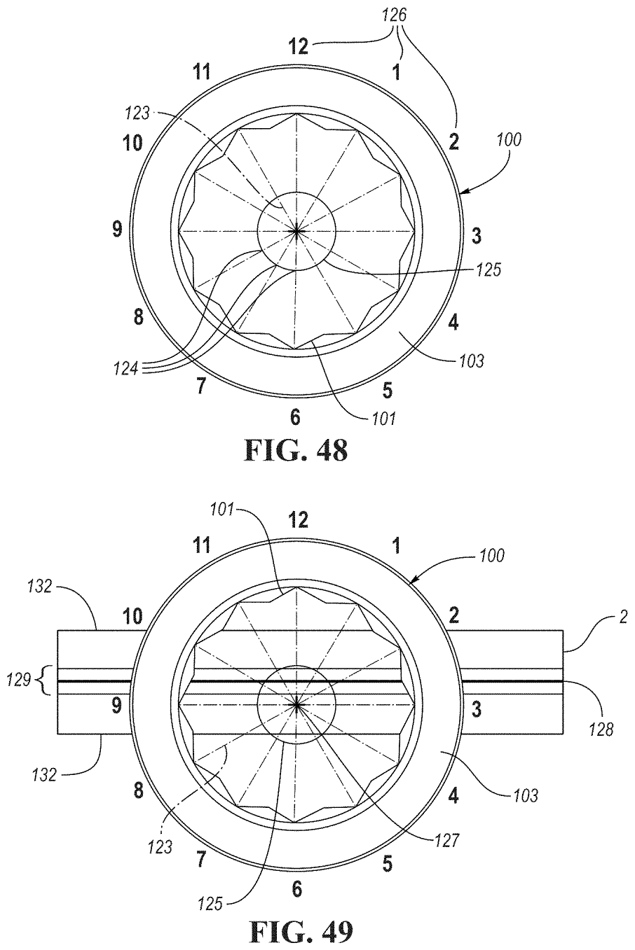

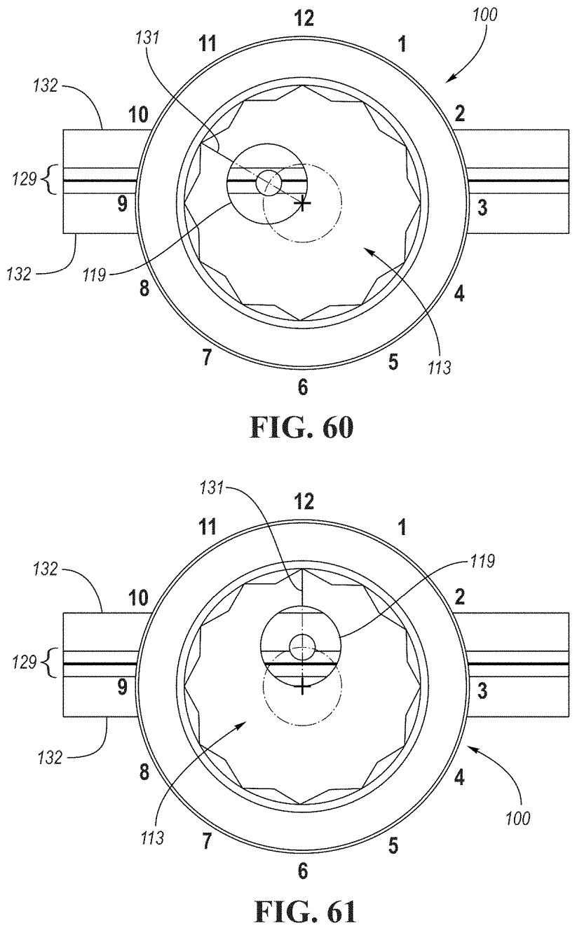

Another embodiment of the implant assembly 114 is shown in FIGS. 48-63. In these figures, the lumen axis of the indexable implant body 113 and the associated microaperture 4 are not co-aligned with the axis of the bone anchor 100. The bone anchor 100 is shown with internal facets 101 and corresponding index marks or indicia 126, shown as a set of twelve for this particular example, although any number of facets 101/index marks 126 is possible, with an increasing amount of facets 101/index marks 126 resulting in an increasing accuracy for targeting the location of the microaperture 4. As shown in FIGS. 48-49, overlaid onto the bone anchor 100 is a schematic map of radial lines 123 having an intersection on the axis 127 of the bone anchor 100 and crossing the upper flange 103 at a regular 30° of separation. The radial lines 123 (corresponding to the index indicia 126) may be identified on the upper flange 103. An offset distance is selected, typically between 1.5 mm and 2 mm from the axis 127 of the bone anchor 100. A circle 125 made of the offset distance radius points is also shown on this map. Where the radial lines 123 intersect the offset circle 125 represents the allowed points 124 of incremental travel of the lumen 102 of the indexing tool 107 and the lumen 119 of the indexable implant body 113 around the axis 127 of the bone anchor 100.

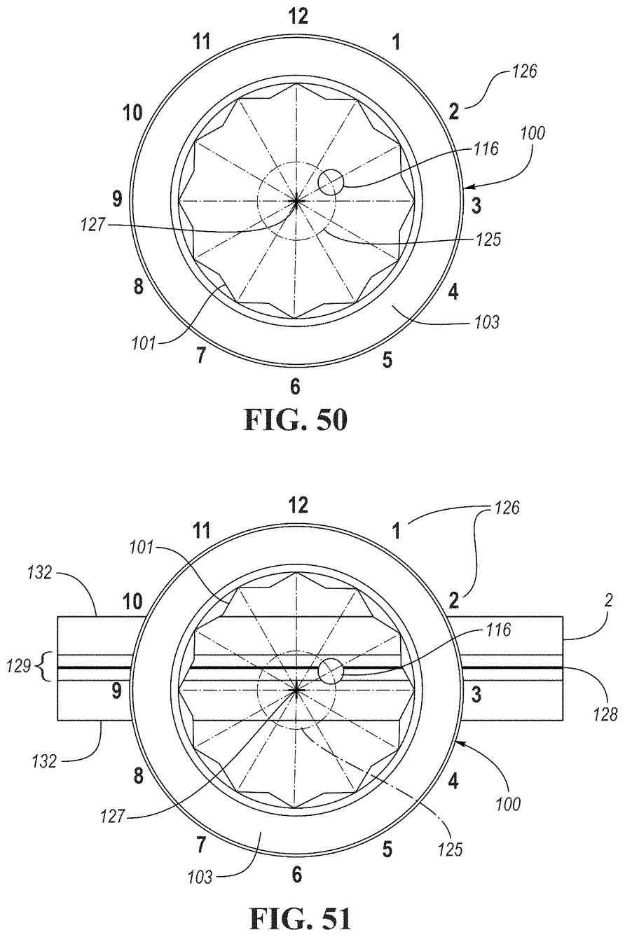

With reference to FIGS. 50-51, this example shows that the axis 127 of the bone anchor 100 and the macroaperture 3 is not centered over the ideal location 129 of the indexable implant body 113 on the DVS 2 and is offset from the longitudinal axis 128 of the DVS 2 by some distance. The width of the DVS 2 is represented by the edges 132 shown. Since the DVS 2 is a “V” shaped structure, it is important that the distal tip 116 (represented as a circle in FIGS. 50-51) of the indexable implant body 113 is not too close to the edge 132 of the DVS 2 or there is the possibility that the distal tip 116 may pass completely through the DVS 2.

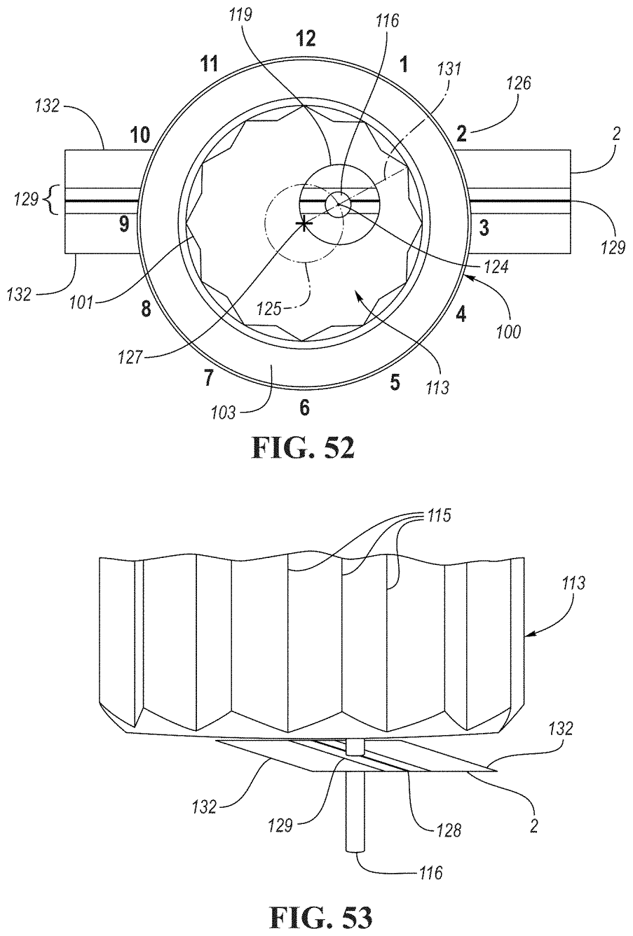

With reference again to the overlaid map of FIG. 51, the intersection of radial position “10”, “11”, “12”, “1” and “2” lines 123 and the radial offset circle 125 fall within an acceptable target range of the ideal location 129 on the DVS 2. The intersection of the radial offset circle 125 and the radial position “2” line 123 is in position for intersecting the ideal location 129 of the DVS 2, as shown in FIG. 52. Thus, the lumen 119 of the indexable implant body 113 (or the lumen 102 of the indexing tool 107) may be aligned with any of the index indicia 126 (e.g. 1-12). When the index mark 131 on the indexable implant body 113 is registered to position “2” on the bone anchor upper flange 103, the lumen 119 is now centered over the ideal location 129 on the DVS 2 and indicates the location where the microaperture 4 should be created.

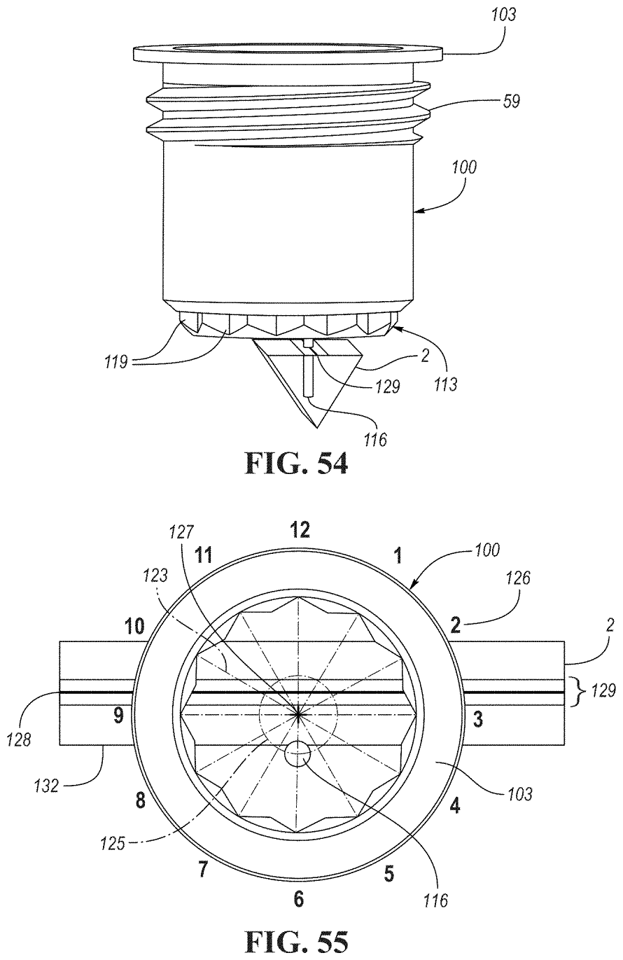

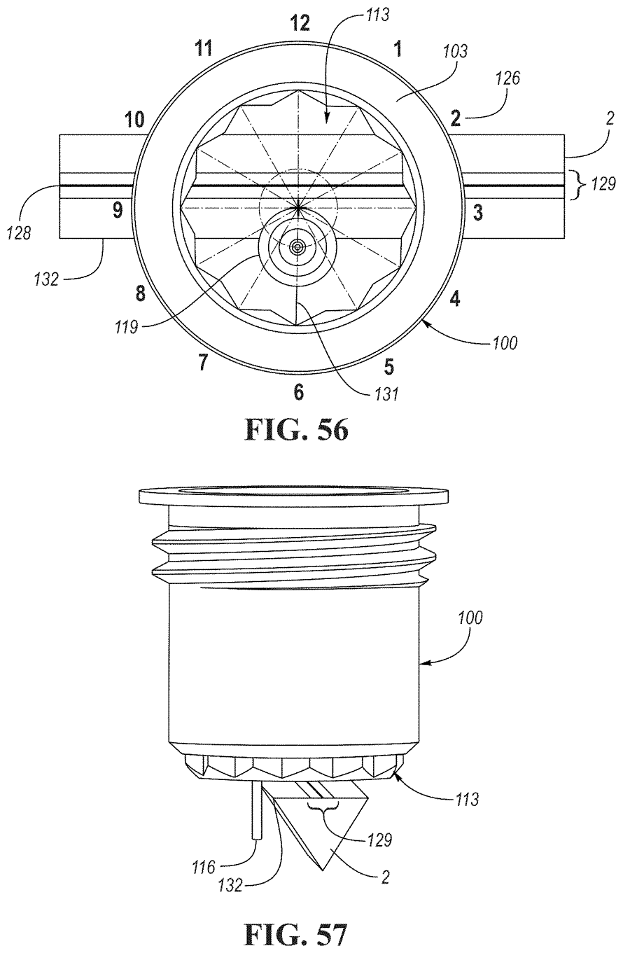

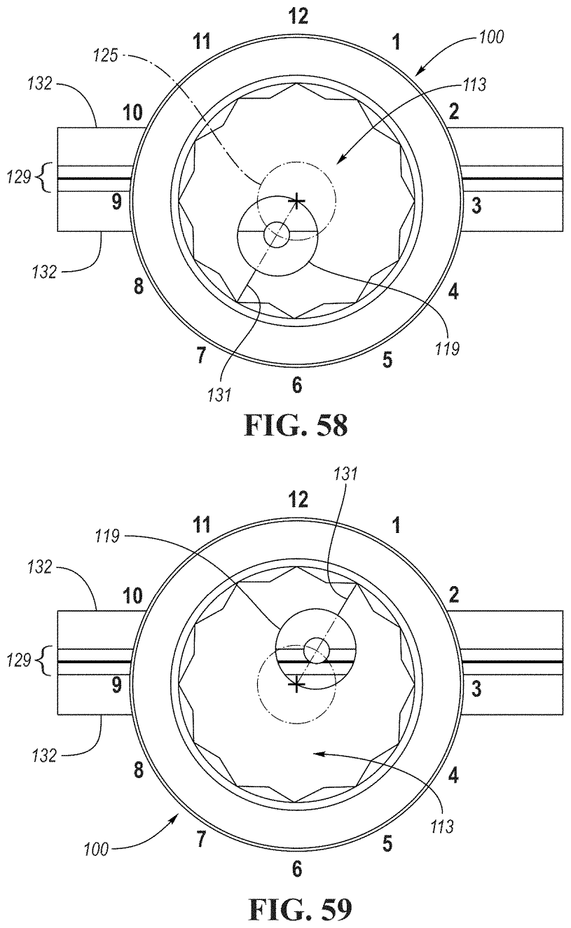

FIGS. 53-54 show a side view of how the distal tip 116 of the indexable implant body 113 will intersect the DVS 2 at the desired location 129. FIG. 55 shows what will be an unacceptable position of the microaperture 4 and the distal tip 116. FIG. 56 shows the indexable implant body 113 in this position with an index mark 131 in position “6”. FIG. 57 shows the side view of the distal tip 116 missing the DVS 2 entirely. FIG. 58 marks the top-down view of this unacceptable position of the microaperture 4. FIGS. 59-61 show other possible positions (“1”, “12”, and “10”) of the index mark 131. While these locations of a microaperture 4 are not ideal, they would still likely be acceptable for the microaperture location.

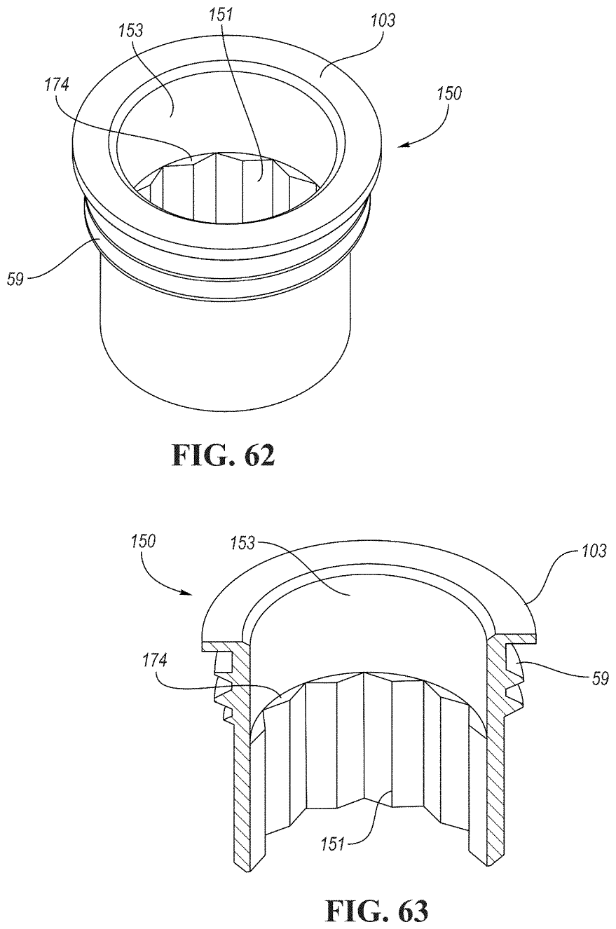

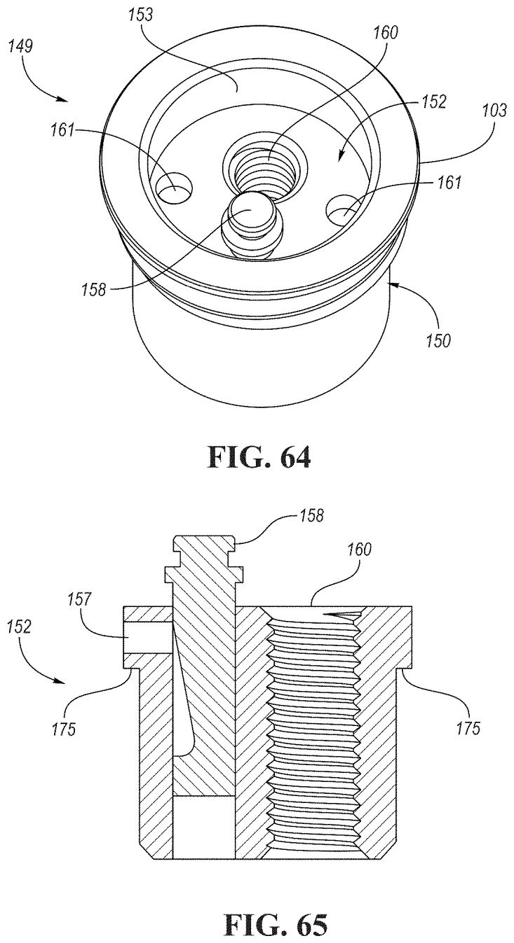

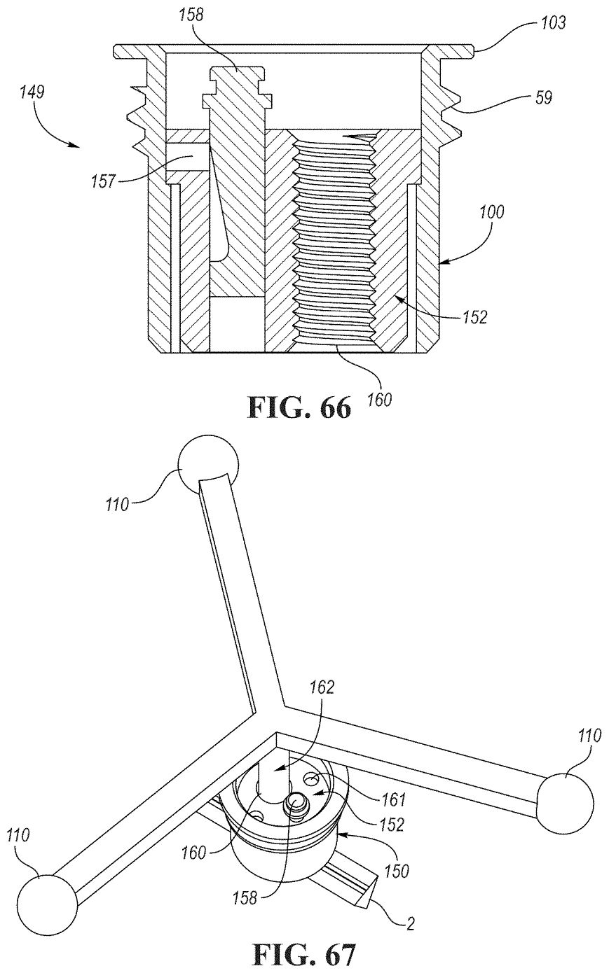

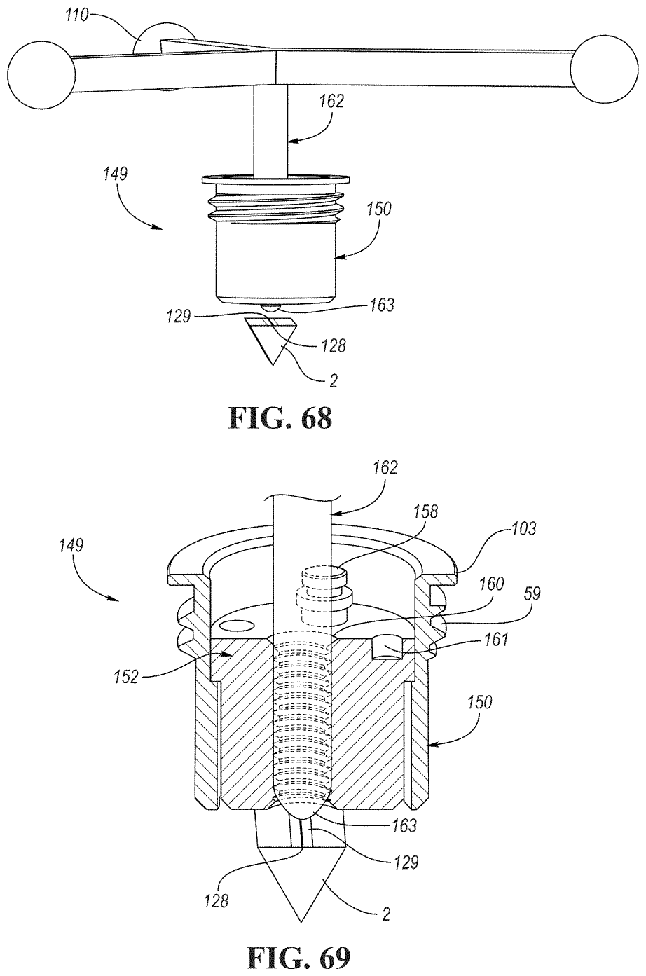

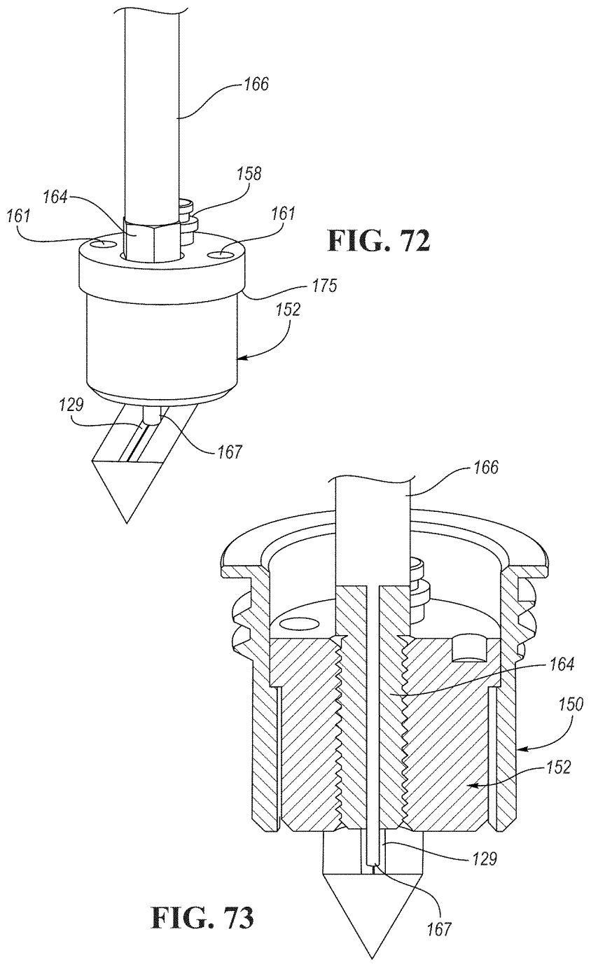

Another embodiment is shown in FIGS. 62-79 which depict an implant assembly 149 with a bone anchor 150 and a rotatable implant holder 152 which share a common axis of rotation, wherein the rotatable implant holder 152 is infinitely rotatable with respect to the bone anchor 150. It is understood that features described above with respect to implant assembly 6, implant assembly 50, implant assembly 80, and implant assembly 114 may be applicable to implant assembly 149, and vice versa. The lumen 160 (FIG. 64) of the rotatable implant holder 152 is offset and is not co-aligned with the longitudinal axis of the bone anchor 150. The bone anchor 150 utilizes offset axial alignment with the microaperture 4 (e.g. 1.5 mm to 2 mm) to allow for more accurate final placement of the microaperture 4, and generally allows for the placement of the microaperture 4 anywhere inside the internal diameter of the bone anchor 150. The bone anchor 150 has a distal internal portion 151 (e.g. with facets, such as of hexangular shape) for engagement by an insertion tool (e.g. a Torx driver; not shown). The bone anchor 150 also has external threads 59. Further, the proximal internal portion 153 of the bone anchor 150 may have a smooth surface with an inner diameter which is larger than the diameter of the distal internal portion 151, defining a shoulder 174 at the junction of the proximal internal portion 153 and the distal internal portion 151.

As shown in FIGS. 64-66, the rotatable implant holder 152 contains its own internal locking cam 158. The rotatable implant holder 152 has an upper flange 175 configured to engage the shoulder 174 of the bone anchor 150, thus automatically setting the insertion depth of the rotatable implant holder 152 at the time of insertion into the bone anchor 150. The rotatable implant holder 152 may have a smooth outer surface, and thus is freely rotatable with respect to the bone anchor 150 with the upper flange 175 moving along the shoulder 174. The combination of the bone anchor 150 and the rotatable implant holder 152 (implant assembly 149) eliminates the need for a separate indexing tool 107 to transfer its coordinates to the implant assembly 149, because the rotational position of the rotatable implant holder 152 can now be selected to be at any point of rotation (infinitely rotatable) within the bone anchor 150, which significantly increases the accuracy for targeting the location of the microaperture 4. The rotatable implant holder 152 contains a lumen 160 which, in the example shown in FIGS. 64, 70 and 75, is threaded to receive a threaded cutting guide 164. The lumen 160 has a similar offset distance (e.g. between 1.5 mm and 2 mm) from the axis of the bone anchor 150.

Referring to FIGS. 67-70, once the bone anchor 150 has been inserted into the cranial bone 1, the rotatable implant holder 152 is inserted into the bone anchor 150 and will rest on the shoulder 174 with its upper flange 175. Then, a navigational or imaging probe 162 may be inserted through the lumen 160 with the distal tip 163 of the probe 162 resting on the remaining inner table 16. Once registered to the navigation system, with the probe 162 still attached and using a pin wrench (not shown) to engage with detents 161 in the top surface of the rotatable implant holder 152, the rotatable implant holder 152 can be rotated with the probe 162 attached until the probe distal tip 163 is aligned with ideal location 129 on the DVS 2. Once the ideal position 129 has been determined, the rotatable implant holder 152 may then be locked both rotationally and vertically to the bone anchor 150 by depressing the locking cam 158 disposed within the rotatable implant holder 152 to engage a compression pin 157, thereby displacing the pin 157 outwardly to engage the bone anchor 150. It is understood other locking mechanisms for fixing the position of the rotatable implant holder 152 to the bone anchor 150 are also fully contemplated. It should be noted that the probe 162 may use a distal tip 163 that also integrates an ultrasound or infrared sensor to help identify the exact location of the DVS 2 via localized anatomic imaging.

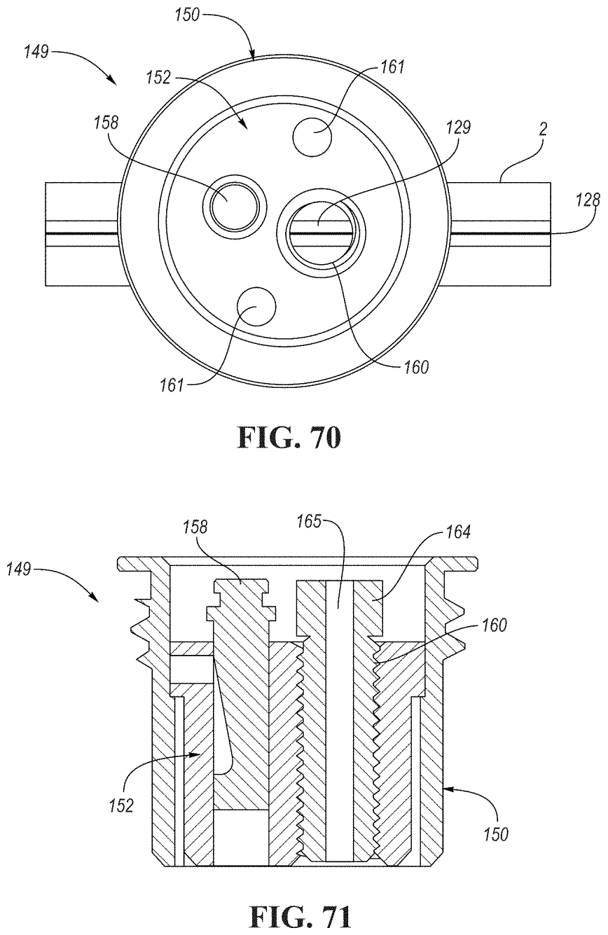

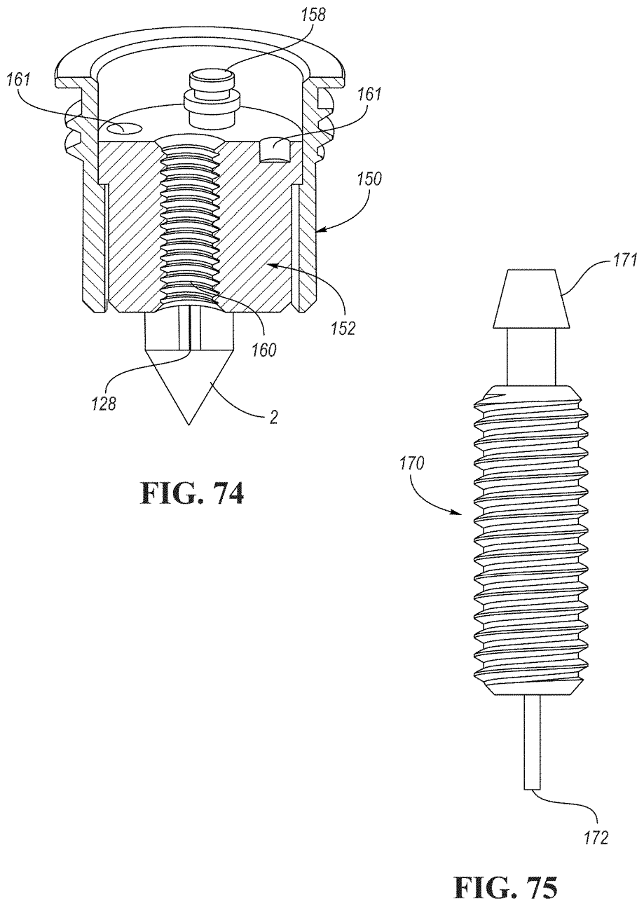

As shown in FIGS. 70-73, once the rotatable implant holder 152 has been locked in the correct position for creating the microaperture 4, a cutting guide 164 may be inserted into the lumen 160 until it engages the remaining inner table 16. The cutting guide 164 includes a lumen 165 to fit the diameter of a desired microaperture cutting tool 166 with a tip 167, such as a drill, end mill, laser, waterjet or other suitable device. The cutting tool 166 may include a shoulder 177 forming a transition from a larger diameter to a smaller diameter of the cutting tool 166, such that as the cutting tool 166 is advanced into the cutting guide 164, the shoulder 177 engages the top of the cutting guide 164 to limit the depth of penetration of the tip 167 into the dura 34. FIG. 74 is a section view of the rotatable implant holder 152 inside the bone anchor 150 with the axis of the lumen 160 directly overlying the longitudinal axis 128 of the DVS 2.

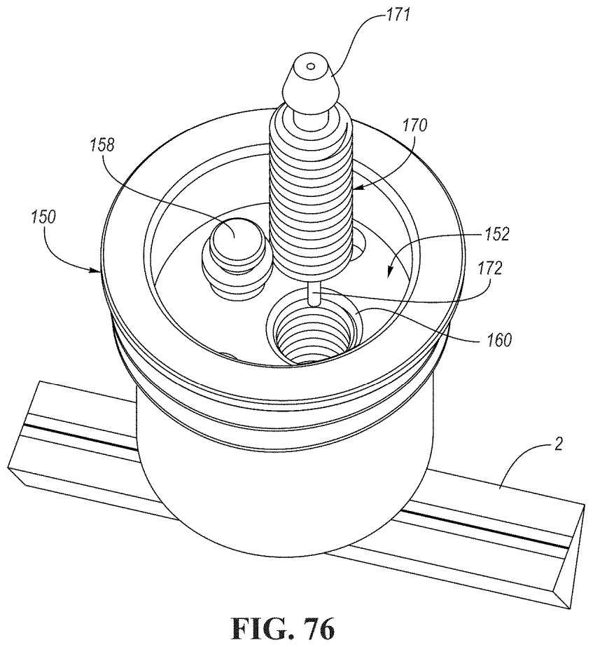

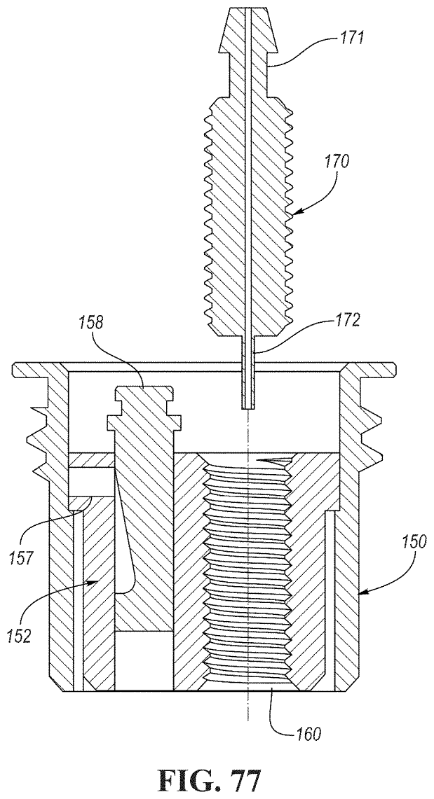

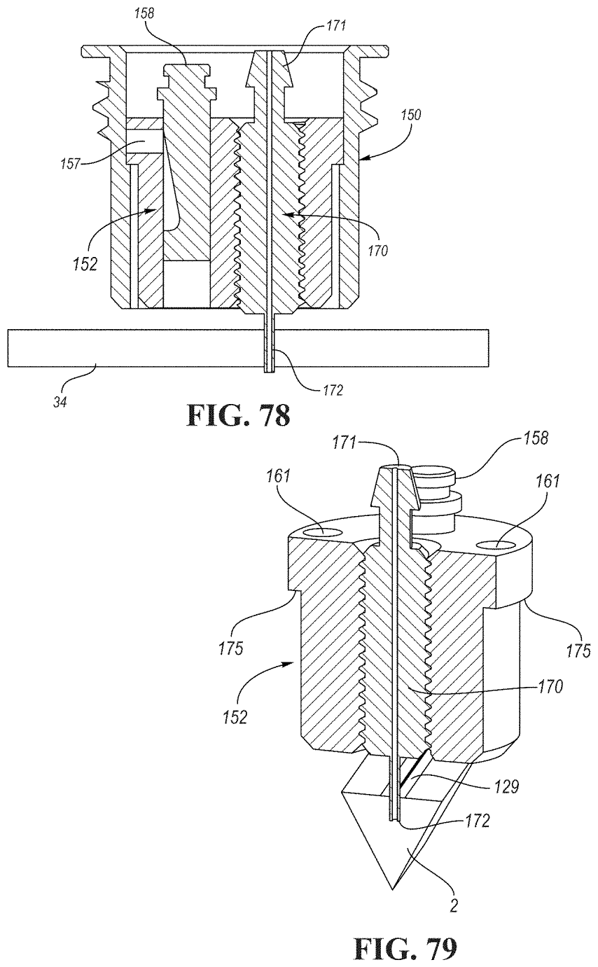

FIG. 75 is a side view of an elongated, hollow implant body 170 to be used with the rotatable implant holder 152. The proximal end 171 of the implant body 170 is configured as a connector to be fluidly and operably coupled with a drainage catheter 54. The distal tip 172 of the implant body 170 is configured to extend through the bone anchor 150 and may be sized to control the flow and back-pressure of the draining CSF. FIG. 76 shows a perspective view of the implant body 170 just above the rotatable implant holder 152. FIG. 77 shows a section view of the implant body 170 just before entering the rotatable implant holder 152, and FIGS. 78-79 show the implant body 170 in final position in the rotatable implant holder 152 and protruding through the ideal location 129 of the superior surface of the DVS 2. The elastomeric catheter 54 coming from the lateral ventricle of the patient (not shown) may then be joined to the implant body 170. At this point, a membrane 29 can be placed over the proximal end of the implant assembly 149 and the surgical site closed.

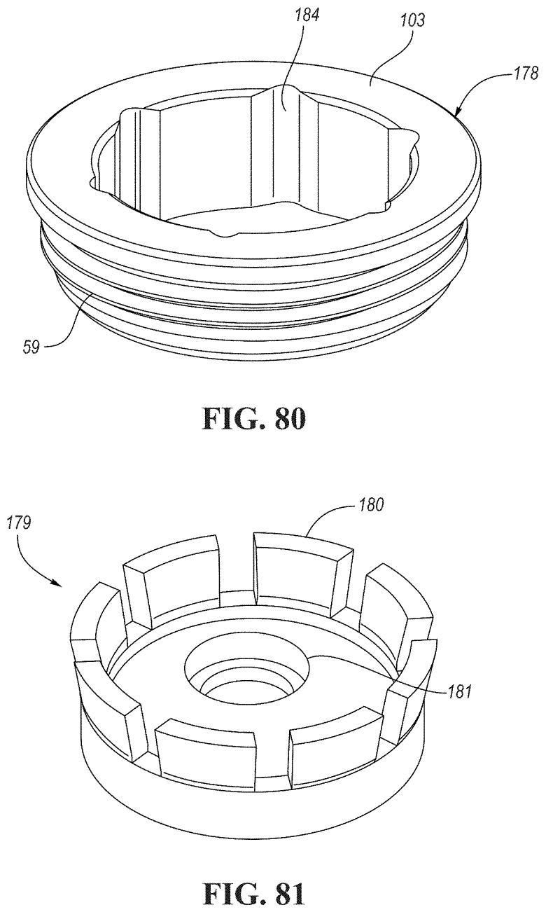

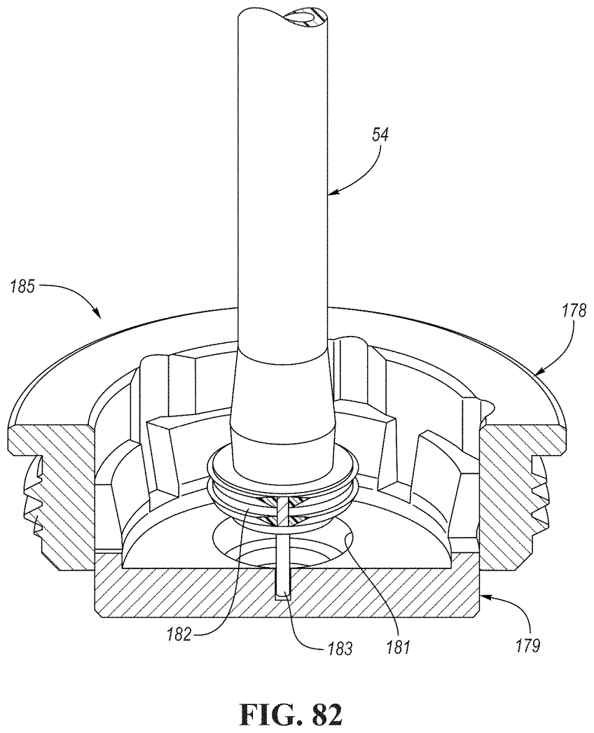

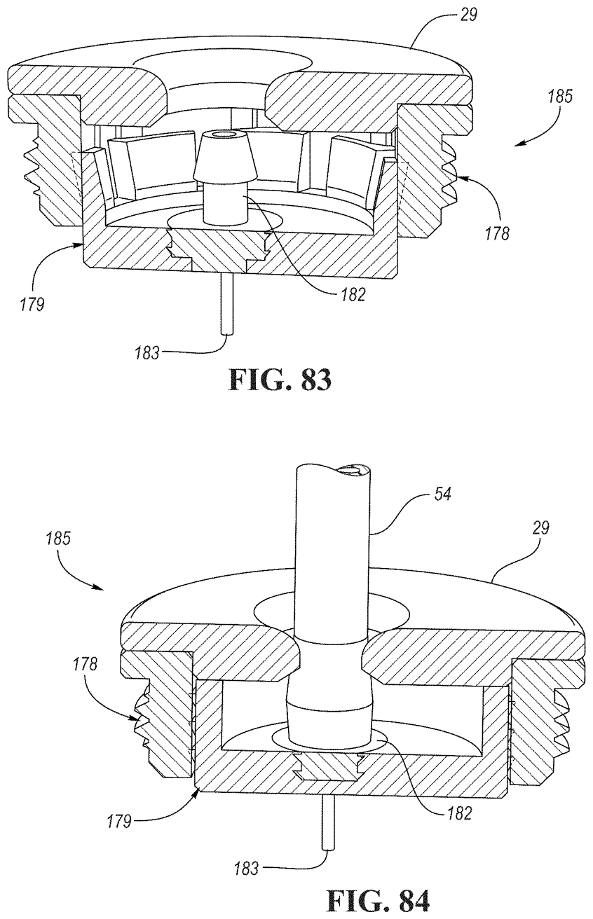

FIG. 80 is a perspective view of another embodiment of a bone anchor 178, which may be selected according to the depth of the macroaperture 3. The bone anchor 178 can be driven into the outer table 15 of the cranial bone 1 using internal facets 184 and secured in the cranial bone 1 via its external threads 59. FIG. 81 is a perspective view of an implant holder 179 selected to match the length of the selected bone anchor 178. The implant holder 179 is configured to be pushed into the bone anchor 178 such that spaced flanges 180 on the implant holder 179 form an interference lock with the bone anchor 178, as shown in FIG. 82. The implant holder 179 has an opening 181 with an axis offset from the longitudinal axis of the bone anchor 178. The implant holder 179 may be rotationally aligned using a navigational probe 162 such that the opening 181 is over the target longitudinal axis 128 of the DVS 2. The implant holder 179 may be pushed downward into a final position wherein its distal surface is just touching the proximal surface of the remaining inner table bone 16. With continuing reference to FIG. 82, an implant body 182 (shown with a catheter 54 attached) may be inserted into the opening 181 of the implant holder 179 until the distal tip 183 of the implant body 182 is positioned through the dura 34 and into the DVS 2. FIG. 83 shows the implant holder 179 in position within the bone anchor 178, with the implant body 182 fully inserted into the implant holder 179 to create an implant assembly 185. Once the implant body 182 is fully seated, a resealable membrane 29 may be passed over the catheter 54 to engage the bone anchor 178, as illustrated in FIG. 84. As above, it is understood that features of implant assembly 6, implant assembly 50, implant assembly 80, implant assembly 114, and implant assembly 149 described herein may also be applicable to implant assembly 185, and vice versa.

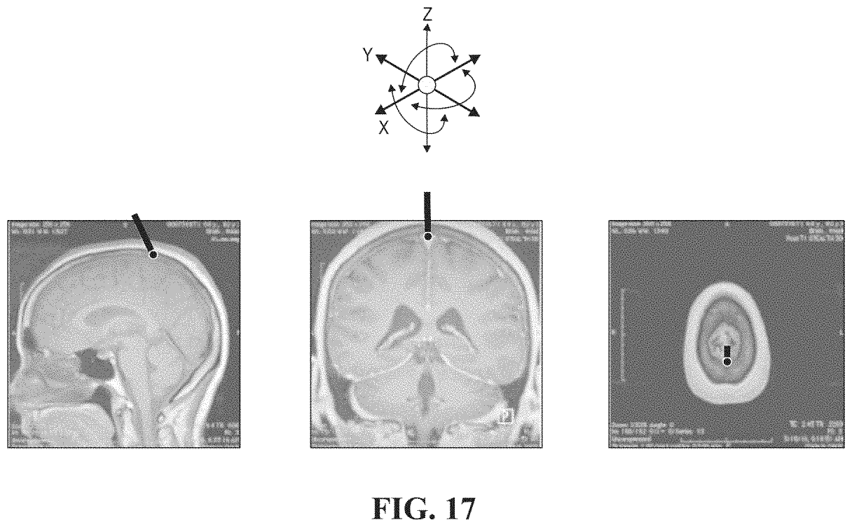

FIG. 17 illustrates a process for identifying a desired location for an implant assembly disclosed herein showing orthogonal coordinates X-Y-Z which define three planes. Each image is taken in one of those planes (i.e. the sagittal view in the X-Z plane, coronal view in the Y-Z plane, and axial view in the X-Y plane). By triangulating the information in these three views, the desired location for a distal tip 36 (marked with a dot and line) of the implant body 30 may be determined. Once the desired location is determined for the distal tip 36, the desired orientation of the implant assembly 6 can be defined by a combination of the X, Y and Z positions of the distal tip 36 and each of the X, Y, and Z roll axes to fully identify the trajectory of the implant assembly 6 through the cranial bone 1. This information can subsequently be used for purposes of guiding the cutting device to create the location and angle of the macroaperture 3 and microaperture 4 through the cranial bone 1 and the final position of the implant assembly 6 in the cranial bone 1 and dura 34.

Prior to inserting an implant assembly of any of the embodiments disclosed herein into a combined macroaperture 3 and microaperture 4, the user may select the most appropriate implant assembly based on, for example, shape, dimensions, and/or function(s). Accordingly, in one or more embodiments, a kit may be provided for facilitating and optimizing implementation of the implant assemblies and methods disclosed herein. For example, the kit may include a variety of differing bone anchors and/or implant bodies, and prior to insertion a user may make a selection of these components based on such features as inner and outer surface shape or texture (e.g. “offset” or “non-offset”), inner and outer size or dimensions (e.g. “long” or “short”), fluid conduction properties, and/or function. For example, the kit may provide a variety of bone anchors and implant bodies each defining a differently configured lumen therethrough (e.g. having differing lumen internal dimensions and differing lumen lengths) and configured to be interchangeably inserted (e.g. so that the internal dimension and length of an implant body received within a bone anchor is independent of dimensions of the outer surface of the bone anchor). It is contemplated that a kit may also include more than one of the same type of component. For example, if more than one combined macroaperture 3 and microaperture 4 are created, and/or during installation or use a component breaks or is dropped, it is advantageous for a kit to include another identical component as a replacement.

The kit may also include a variety of other components such as, but not limited to, a razor, antiseptic, bandages, fiducial markers, a cutting device, and resealable membranes. A user guide, instructions, and/or indicia associated with the components, either on or in the components or associated packaging, may be provided for assisting in selecting any of the foregoing components from a kit. The indicia may take the form of a label (e.g. descriptive, coding, or barcode labels), numbering, instructions, and/or color-coding.

Also, a kit for use in establishing a practice may also include reusable/durable components, such as an imaging device and related equipment (e.g. CT, MRI, RF, EM, camera, fiducial markers) and a computer system including a user interface for referencing a patient's anatomy to a surgical navigation system for guiding the creation of the macroaperture and microaperture as selected by a surgeon. The kit may also include components configured to provide chemical analysis, assess hardness and or shape, one or more cutting devices, and a mounting system for the cutting device, such as a robotic arm, stereotactic frame or other member for mechanically positioning the cutting device (e.g. positioning a laser so that the laser aiming point is at an appropriate position from the laser outlet to provide a desired focal point and beam width). Another type of kit may include components that might be used in the surveillance, maintenance or replacement of a patient's existing components (e.g. bone anchor, implant body, resealable membrane, flushing device, implant body/removal tool, etc.).

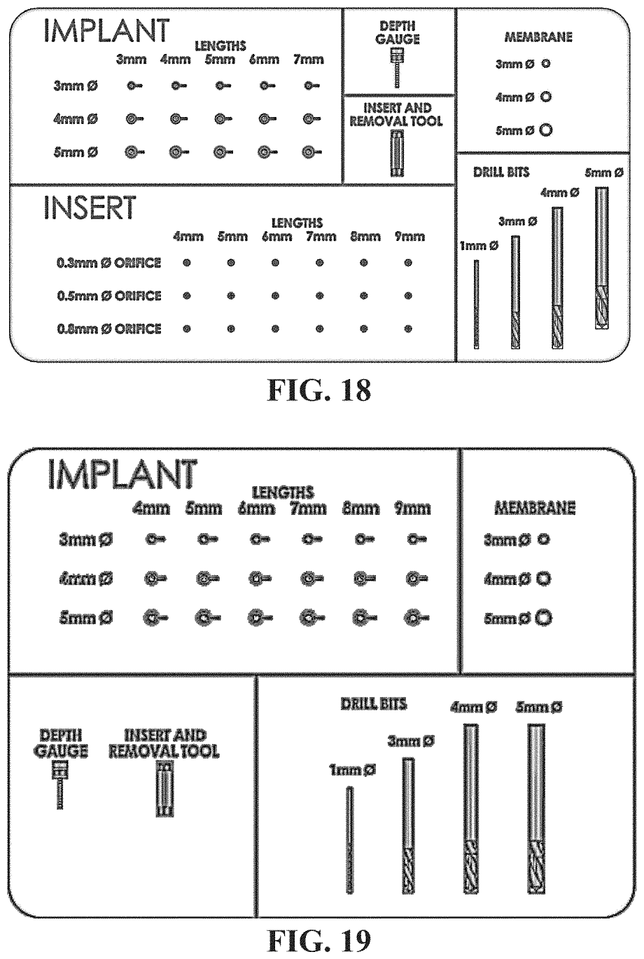

FIG. 18 is a schematic representation of a kit according to one or more embodiments which may be applicable to any of the implant assemblies described herein. The components represented in FIG. 18, which may be on a surgical tray, include drill bits of various sizes/shapes, a depth gauge for measuring the guiding macroaperture 3 and microaperture 4 to assist the surgeon in selecting a bone anchor (implant), an array of implant bodies (insert) of varying lengths/diameters/functions, an insert/removal tool for use in inserting the implant assembly, and a plurality of resealable membranes. FIG. 19 is a schematic representation of a second type of kit according to one or more embodiments. The embodiment depicted in FIG. 19 may be on a surgical tray and includes an array of bone anchors (implant) of a variety of sizes. It is understood that all of the dimensions depicted in FIGS. 18 and 19 are for illustrative purposes only, and other dimensions, shapes, and functions of components may be included in a kit depending on the particular needs of the patient.

Embodiments disclosed herein offer substantial advantages in safely and straightforwardly providing medical aid to humans and non-human animals including medical diagnosis, treatment and/or monitoring. This includes facilitating the use of medical aids such as probes, electrodes, sensors, diagnostic devices, and/or fluid conductors for permitting fluid to move to or from the implant assembly. More specifically, fluid conductors may include a fluid reservoir and a conduit which defines an internal passage having a proximal end and a distal end. The distal end of the conduit may be connected to the fluid reservoir and the proximal end of the conduit may be connected to the bone anchor, wherein fluid may flow between (to or from) the implant assembly and the fluid reservoir via the conduit. For example, fluid may move from the implant assembly to the fluid reservoir when it is desired to drain fluid from a patient, or fluid may move from the fluid reservoir to the implant assembly, and hence to the DVS, when it is desired to administer medication or flush the subject components.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. An implant assembly for accessing a dural venous sinus (DVS) in a cranial bone, the implant assembly configured to be received in a macroaperture created in the cranial bone adjacent an outer table thereof and in a connected microaperture of smaller diameter than the macroaperture created in the cranial bone and extending toward an inner table thereof, the implant assembly comprising:
a bone anchor configured to be received in at least the macroaperture, the bone anchor having an upper portion with a larger diameter than a lower portion thereof, the bone anchor having a lumen therethrough; and
an implant body configured to be received in the lumen of the bone anchor and extend through the bone anchor into the microaperture, the implant body having an upper portion with a larger diameter than a lower portion thereof, the implant body having a lumen for providing access therethrough to the DVS,
wherein an inner surface of the bone anchor and an outer surface of the implant body have complementary shapes to secure the implant body to the bone anchor via an interference fit.

2. The implant assembly of claim 1, wherein the bone anchor has a substantially constant wall thickness along a length thereof.

3. The implant assembly of claim 1, wherein an inner surface of the bone anchor includes a shelf configured to receive the upper portion of the implant body thereon.

4. The implant assembly of claim 1, wherein a distal tip of the implant body extends beyond a distal end of the bone anchor when the implant body is received in the bone anchor.

5. The implant assembly of claim 4, wherein a lower portion of the implant body includes at least one port adjacent the distal tip.

6. The implant assembly of claim 4, wherein the distal tip of the implant body includes a sensor.

7. The implant assembly of claim 1, wherein the bone anchor includes a top portion configured to receive a sensor.

8. The implant assembly of claim 1, wherein the implant body has a top portion with slots for facilitating insertion, positioning, and removal of the implant body in the bone anchor.

9. The implant assembly of claim 1, wherein an external shape of the implant assembly is complementary to a shape of the macroaperture and microaperture.

10. The implant assembly of claim 1, wherein the bone anchor has an outer surface which includes at least one engaging member configured to engage with the macroaperture.

11. The implant assembly of claim 1, further comprising a resealable membrane configured to be received on the implant assembly, wherein the lumen of the implant body is accessible therethrough.

12. The implant assembly of claim 1, further comprising a fluid coupler configured to be received on a proximal end of the implant assembly and operably connected to a catheter for fluid flow between a fluid source and the implant assembly.

13. The implant assembly of claim 1, wherein the bone anchor includes a conduit integrally formed therewith for fluid flow between a fluid source and the implant assembly.

14. The implant assembly of claim 13, further comprising a cap configured to be received in a proximal opening of the bone anchor, the cap including a lumen and a plurality of orifices of different dimensions in fluid communication with the lumen, wherein the lumen of the bone anchor is accessible via the cap and the cap is configured to be rotatable so that one of the plurality of orifices aligns with the conduit to select a desired fluid flow rate through the implant assembly.

15. The implant assembly of claim 1, wherein the implant assembly is constructed from at least one of a metal, plastic, ceramic, resorbable compound, biologic tissue, bone substitute, or material derived from a cadaver.

16. The implant assembly of claim 1, wherein the implant assembly is coated with a low friction antithrombogenic material or a material which limits bony or other tissue ingrowth.

17. An implant assembly for accessing a dural venous sinus (DVS) in a cranial bone, the implant assembly configured to be received in a macroaperture created in the cranial bone adjacent an outer table thereof and a connected microaperture of smaller diameter than the macroaperture created in the cranial bone and extending toward an inner table thereof, the implant assembly comprising:
a generally cylindrical bone anchor configured to be received in the macroaperture, the bone anchor having external threads configured to engage the macroaperture, the bone anchor having a lumen with internal threads; and
an implant body configured to be received in the lumen of the bone anchor and extend through the bone anchor into the microaperture, the implant body having external threads configured to be received by the internal threads to locate and secure the implant body in the bone anchor, the implant body having a lumen for providing access therethrough to the DVS.

18. The implant assembly of claim 17, wherein the bone anchor has an internal stop surface configured to engage the implant body when received in the bone anchor, the internal stop surface having an opening therein for receiving the implant body therethrough.

19. The implant assembly of claim 18, wherein the implant body has a proximal portion and an elongated distal portion, wherein the distal portion has a diameter smaller than a diameter of the proximal portion, wherein when the implant body is received in the bone anchor, the proximal portion is configured to engage the internal stop surface and the distal portion is configured to be received through the opening.

20. The implant assembly of claim 18, wherein the implant body is tubular with a constant cross-sectional area, the implant body having a stop plate configured to engage the internal stop surface of the bone anchor when the implant body is received in the bone anchor.

21. The implant assembly of claim 17, further comprising a hollow needle arranged to be received through the implant assembly, the needle including a sharp distal tip, markings to indicate depth of insertion, and a stop member configured to engage the implant assembly to limit depth of insertion of the needle through the implant assembly.

22. The implant assembly of claim 17, wherein a lower portion of the implant body includes at least one port adjacent a distal tip of the implant body.

23. The implant assembly of claim 17, wherein a distal tip of the implant body includes a sensor.

24. The implant assembly of claim 17, further comprising a resealable membrane configured to be received on the implant assembly, wherein the lumen of the implant body is accessible therethrough.

25. The implant assembly of claim 17, wherein a proximal end of the implant body includes a connector configured to be coupled to a catheter or a fluid coupler.

26. The implant assembly of claim 17, further comprising a fluid coupler configured to be received on a proximal end of the implant assembly and operably connected to a catheter for fluid flow between a fluid source and the implant assembly.

27. The implant assembly of claim 17, wherein the implant assembly is constructed from at least one of a metal, plastic, ceramic, resorbable compound, biologic tissue, bone substitute, or material derived from a cadaver.

28. The implant assembly of claim 17, wherein the implant assembly is coated with a low friction antithrombogenic material or a material which limits bony or other tissue ingrowth.

29. An implant assembly for accessing a dural venous sinus (DVS) in a cranial bone, the implant assembly configured to be received in a macroaperture created in the cranial bone adjacent an outer table thereof and a connected microaperture of smaller diameter than the macroaperture created in the cranial bone and extending toward an inner table thereof, the implant assembly comprising:
a generally cylindrical bone anchor configured to be received in the macroaperture, the bone anchor having external threads configured to engage the macroaperture, the bone anchor having a lumen with internal facets; and
a generally cylindrical implant body configured to be received in the lumen of the bone anchor and extend through the bone anchor into the microaperture, the implant body having external facets configured to be received by the internal facets of the bone anchor such that the implant body movable with respect to the bone anchor in fixed angular steps, the implant body having a lumen for providing access therethrough to the DVS.

30. The implant assembly of claim 29, wherein the bone anchor has an upper flange configured to limit a depth of insertion of the bone anchor in the cranial bone.

31. The implant assembly of claim 29, wherein the bone anchor includes index marks thereon.

32. The implant assembly of claim 29, wherein the lumen of the implant body has a longitudinal axis that is offset from a longitudinal axis of the implant body, the implant body having index marks thereon for aligning the implant body with the bone anchor.

33. The implant assembly of claim 29, further comprising an indexing tool having external facets configured to be received by the internal facets of the bone anchor, a lumen extending therethrough with a longitudinal axis that is offset from a longitudinal axis of the indexing tool, and index marks thereon for aligning the indexing tool with the bone anchor, the indexing tool movable with respect to the bone anchor in fixed angular steps.

34. The implant assembly of claim 29, further comprising a locking ring for securing the implant body to the bone anchor.

35. The implant assembly of claim 29, wherein a distal tip of the implant body includes a sensor.

36. The implant assembly of claim 29, further comprising a resealable membrane configured to be received on the implant assembly, wherein the lumen of the implant body is accessible therethrough.

37. The implant assembly of claim 29, wherein a proximal end of the implant body includes a connector configured to be coupled to a catheter or a fluid coupler.

38. The implant assembly of claim 29, further comprising a fluid coupler configured to be received on a proximal end of the implant assembly and operably connected to a catheter for fluid flow between a fluid source and the implant assembly.

39. The implant assembly of claim 29, wherein the implant assembly is constructed from at least one of a metal, plastic, ceramic, resorbable compound, biologic tissue, bone substitute, or material derived from a cadaver.

40. The implant assembly of claim 29, wherein the implant assembly is coated with a low friction antithrombogenic material or a material which limits bony or other tissue ingrowth.

41. An implant assembly for accessing a dural venous sinus (DVS) in a cranial bone, the implant assembly configured to be received in a macroaperture created in the cranial bone adjacent an outer table thereof and a connected microaperture of smaller diameter than the macroaperture created in the cranial bone and extending toward an inner table thereof, the implant assembly comprising:
a generally cylindrical bone anchor configured to be received in the macroaperture, the bone anchor having external threads configured to engage the macroaperture, the bone anchor having a lumen with a proximal internal portion and a distal internal portion, the proximal internal portion having a diameter larger than a diameter of the distal internal portion to define a shoulder therebetween;
a generally cylindrical implant holder configured to be received in the lumen of the bone anchor, the implant holder including an upper flange configured to engage and be movable along the shoulder such that the implant holder is infinitely rotatable with respect to the bone anchor, the implant holder having a lumen extending therethrough; and
an implant body configured to be received in the lumen of the implant holder and extend through the implant holder into the microaperture, the implant body having a lumen for providing access therethrough to the DVS.

42. The implant assembly of claim 41, wherein the implant body includes a proximal end having a connector configured to be coupled to a catheter or a fluid coupler.

43. The implant assembly of claim 41, wherein the lumen of the implant holder has a longitudinal axis that is offset from a longitudinal axis of the bone anchor.

44. The implant assembly of claim 41, wherein the distal internal portion of the bone anchor has internal facets and the proximal internal portion of the bone anchor has a smooth surface.

45. The implant assembly of claim 41, wherein the implant holder includes an internal locking cam and associated compression pin, wherein engagement of the locking cam displaces the pin outwardly to engage the proximal internal portion of the bone anchor and fix a position of the implant holder with respect to the bone anchor.

46. The implant assembly of claim 41, wherein the lumen of the implant holder is threaded and wherein the implant body is threaded.

47. The implant assembly of claim 46, further comprising a threaded cutting guide configured to be received in the threaded lumen of the implant holder.

48. The implant assembly of claim 41, wherein the bone anchor has an upper flange configured to limit a depth of insertion of the bone anchor in the cranial bone.

49. The implant assembly of claim 41, wherein a distal tip of the implant body includes a sensor.

50. The implant assembly of claim 41, further comprising a resealable membrane configured to be received on the implant assembly, wherein the lumen of the implant body is accessible therethrough.

51. The implant assembly of claim 41, further comprising a fluid coupler configured to be received on a proximal end of the implant assembly and operably connected to a catheter for fluid flow between a fluid source and the implant assembly.

52. The implant assembly of claim 41, wherein the implant assembly is constructed from at least one of a metal, plastic, ceramic, resorbable compound, biologic tissue, bone substitute, or material derived from a cadaver.

53. The implant assembly of claim 41, wherein the implant assembly is coated with a low friction antithrombogenic material or a material which limits bony or other tissue ingrowth.
